(19) United States
(12) Patent Application Publication
(10) Pub. No.: US 2026/0235592 A1
(43) Pub. Date: Aug. 13, 2026

(54) REACTION PARTICLES WITH TARGET-SPECIFIC LUMINESCENT DETECTION

(71) Applicants: Promega Corporation, Madison, WI (US); Blink AG, Jena (DE)

(72) Inventors: Debayan DE BAKSHI, Madison, WI (US); Thomas MACHLEIDT, Madison, WI (US); Lance ENCELL, Madison, WI (US); Wenhui ZHOU, Madison, WI (US); Zhiyang ZENG, Madison, WI (US); Rachel FRIEDMAN OHANA, Madison, WI (US); Mark KLEIN, Madison, WI (US); Elizabeth VU, Madison, WI (US); Karllyn PORTER, Madison, WI (US); Juliano ALVES, Madison, WI (US); Hicham ZEGZOUTI, Madison, WI (US); Mike VALLEY, Madison, WI (US); Jolanta VIDUGIRIENE, Madison, WI (US); Eugen ERMANTRAUT, Jena (DE); Lennart GURA, Jena (DE); Jens TUCHSCHERER, Jena (DE); Theresa LIEBE, Jena (DE); Thomas ELLINGER, Jena (DE); Stephan HUBOLD, Jena (DE)

(21) Appl. No.: 19/467,457

(22) Filed: Feb. 2, 2026

Related U.S. Application Data
(60) Provisional application No. 63/753,781, filed on Feb. 4, 2025.

(51) Int. Cl. G01N 33/542; G01N 21/64; G01N 21/76; G01N 33/543

(52) U.S. Cl. CPC G01N 33/542; G01N 21/6428; G01N 21/6458; G01N 21/763; G01N 33/54313; G01N 2021/6441

(57) ABSTRACT

Provided herein are assay particles comprising a matrix material, interstitial void volume, and reagents for target-specific luminescent detection.

### CROSS REFERENCE

This application claims the benefit of U.S. Provisional Patent Application No. 63/753,781, filed Feb. 4, 2025, which is incorporated by reference herein in its entirety.

### SEQUENCE LISTING

The text of the computer readable sequence listing filed herewith, titled “PRMG_43687_202_SequenceListing.xml”, created Feb. 2, 2026, having a file size of 8,903 bytes, is hereby incorporated by reference in its entirety.

### FIELD

Provided herein are assay particles comprising a matrix material, interstitial void volume, and reagents for target-specific luminescent detection.

### BACKGROUND

Multiplexed profiling of target analytes refers to the simultaneous measurement of multiple biological or chemical markers (analytes) in a single sample. Such techniques allow for high-throughput analysis, improving both efficiency and accuracy in various scientific and medical fields.

### SUMMARY

Provided herein are assay particles comprising a matrix material, interstitial void volume, and reagents for target-specific luminescent detection.

Various aspects of the present technology are summarized below. Although listed as separated aspects, the embodiments described below may be combined and adapted in any suitable manner.

In a first aspect, an assay particle is provided. In some embodiments, provided herein are compositions comprising an assay particle, wherein the assay particle is porous, comprises a matrix material, and comprises an interstitial void volume suitable to receive a sample; wherein the assay particle comprises a target-specific luminescent detection agent tethered to the matrix material; wherein the target-specific luminescent detection agent comprises: (i) a target-specific interaction moiety, and (ii) a luminescent reporter capable of producing a luminescent signal; wherein interaction of the target-specific interaction moiety with the target analyte results in a change in the luminescent signal; and the assay particle further comprising an assay-particle-identifying physical characteristic that is detectable and independent of the luminescent reporter.

In some embodiments, the assay particle is a microparticle. In some embodiments, the assay particle is a bead. In some embodiments, the bead is a microbead.

In some embodiments, the matrix material comprises one or more biocompatible polymers. In some embodiments, the one or more biocompatible polymers are natural polymers selected from agarose, chitosan, alginate, chitin, gelatin, carrageenan, collagen, fucoidan, cellulose, laminaran, fibrin, hyaluronic acid, carboxymethyl cellulose, starch, pectin, keratin, chondroitin sulfate, pullulan, xanthan gum, arabic gum, ghatti gum, guar gum, locust bean gum, tragacanth gum, karaya gum, inulin, and albumin. In some embodiments, the one or more biocompatible polymers are synthetic polymers selected from polylactic acid (PLA), poly(lactic-co-glycolic acid) (PLGA), polycaprolactone (PCL), polyethylene glycol (PEG), polyvinyl alcohol (PVA), polymethyl methacrylate (PMMA), polyurethane (PU), poly(D,L-lactic acid) (PDLLA), polyethylene-co-vinyl acetate (EVA), poly(N-isopropylacrylamide) (PNIPAAm), polysiloxane (Silicone), poly(glycolic acid) (PGA), poly(ε-caprolactone-co-lactide) (PCLLA), poly(3-hydroxybutyrate) (PHB), polyvinylidene fluoride (PVDF), poly(alkylene oxides), and citric acid based polymers (e.g., polyoctanediol). In some embodiments, the one or more biocompatible polymers comprise natural and synthetic polymers.

In some embodiments, the matrix material is liquefiable. In some embodiments, the matrix material is liquefied upon the application of heat, light, a triggering agent (e.g., small molecule agent), electromagnetic radiation, and/or an enzyme. In some embodiments, the matrix material is liquefied upon a change of pH, redox potential, ionic strength, temperature, magnetic field, and/or electromagnetic radiation.

In some embodiments, the assay particle is prefabricated. In some embodiments, the assay particle is prefabricated by any one or more of solvent casting, electrospinning, spray-drying, emulsion, mechanical grinding, phase separation, electrowriting, coacervation, spheronization, and lyophilizing. In some embodiments, the assay particle is lyophilized or dehydrated. In some embodiments, the assay particle is suitable for rehydration upon receiving a sample. In some embodiments, the assay particle forms a porous hydrogel upon receiving a sample, and wherein the sample fills the interstitial void volume. In some embodiments, the assay particle is fully or partially hydrated.

In some embodiments, the assay particle is capable of receiving a sample, and wherein the sample fills the interstitial void volume. In some embodiments, the sample fills the interstitial void volume within the porous matrix when the assay particle receives the sample. In some embodiments, the interstitial void volume is suitable for receiving and containing molecules and/or macromolecules, which are present within the sample. In some embodiments, the interstitial void volume provides a reaction space for conducting assays to detect a target analyte in a sample.

In some embodiments, the assay particle is dispersible in a non-aqueous medium. In some embodiments, upon dispersion in the non-aqueous medium, the interstitial void volume provides an isolated reaction space for conducting assays. In some embodiments, upon receipt of a first sample and, thereupon, dispersion in the non-aqueous medium, the isolated reaction space is suitable to confine a portion of the first sample within the isolated reaction space and prevent the entrance into the isolated reaction space of a second sample. In some embodiments, upon dispersion in a non-aqueous medium, the isolated reaction space is suitable to confine components/reagents within the isolated reaction space and prevent the entrance into the isolated reaction space external of components, reagents, sample, etc. In some embodiments, external components, reagents, sample, etc., may be added to an isolated reaction space (e.g., particles dispersed in non-aqueous medium) using a suitable carrier (e.g., components, reagents, sample, etc., in a non-aqueous carrier). In some embodiments, the target-specific luminescent detection agent is contained within the interstitial void volume. In some embodiments, the target-specific luminescent detection agent is tethered to the matrix material. In some embodiments, the target-specific luminescent detection agent is covalently tethered to the matrix material. In some embodiments, the target-specific luminescent detection agent is reversibly tethered to the matrix material. In some embodiments, the target-specific luminescent detection agent is reversibly tethered to the matrix material by a cleavable linker. In some embodiments, the cleavable linker is light, chemical, heat, pH, or enzymatically cleavable. In some embodiments, the target-specific luminescent detection agent is reversibly tethered to the matrix material by a noncovalent affinity interaction.

In some embodiments, the luminescent reporter is a bioluminescent reporter. In some embodiments, the bioluminescent reporter is a bioluminescent protein. In some embodiments, the bioluminescent protein is a luciferase. In some embodiments, the luminescent reporter is a chemiluminescent reporter. In some embodiments, the chemiluminescent reporter comprises horseradish peroxidase, alkaline phosphatase, lactase, glucose oxidase, catalase, acid phosphatase, nitrate reductase, glutathione peroxidase, or choline oxidase. In some embodiments, the luminescent reporter is a fluorescent reporter. In some embodiments, the fluorescent reporter is a fluorescent protein. In some embodiments, the luminescent reporter is not a fluorescent reporter.

In some embodiments, the luminescent reporter is a luminescent complex comprising first and second components. In some embodiments, the first and second components are structurally complementary components of a bioluminescent or fluorescent complex. In some embodiments, the first and second components are a resonance energy transfer (RET) pair (e.g., FRET pair, BRET pair, etc.). In some embodiments, the first and second components are tethered together directly or by a linker.

In some embodiments, the assay particle further comprises a secondary detection agent, wherein the secondary detection agent comprises (i) a secondary reporter and (ii) (A) a secondary target-specific interaction moiety (e.g., secondary binding moiety) or (B) a target mimetic. In some embodiments, the luminescent reporter and the secondary reporter comprise a BRET pair (e.g., the luminescent reporter is a bioluminescent protein (e.g., luciferase)), and the secondary reporter is a fluorophore. In some embodiments, the luminescent reporter and the secondary reporter comprise a FRET pair (e.g., the luminescent reporter is a fluorescent protein, and the secondary reporter is a fluorophore). In some embodiments, the luminescent reporter and secondary reporter are first and second components of a complementation-dependent bioluminescent complex.

In some embodiments, the secondary detection agent comprises a secondary target-specific interaction moiety, and the target-specific interaction moiety and the secondary target-specific interaction moiety bind to separate locations on the target.

In some embodiments, the secondary detection agent comprises a target mimetic, wherein the target mimetic is suitable for binding to the target-specific interaction moiety. In some embodiments, the target mimetic is suitable for competing with the target analyte for binding to the target-specific interaction moiety.

In some embodiments, the change in the luminescent signal is an increase in signal, a decrease in signal, generation of a signal, elimination of a signal, generation of resonance energy transfer, increase in resonance energy transfer, decrease in resonance energy transfer, or elimination of resonance energy transfer.

In some embodiments, the target analyte is a small molecule, a peptide, a protein, a nucleic acid, a lipid, a viral particle, or an antibody.

In some embodiments, the target-specific interaction moiety is a target-specific binding agent. In some embodiments, the target-specific binding agent is an antibody, an antibody fragment, a natural target binding domain, an affinity molecule, a computationally derived binding domain, an affimer, an aptamer, a DARPin, a ligand, or a protein. In some embodiments, the target-specific interaction moiety is a ligand for the target analyte. In some embodiments, the target analyte is a ligand for the target-specific interaction moiety.

In some embodiments, an assay particle further comprises one or more magnetic particles on, within, and/or tethered to the assay particle. In some embodiments, the magnetic particle is a magnetic microparticle or magnetic microbead. In some embodiments, the magnetic particle is tethered to the matrix material. In some embodiments, the magnetic particle is within the interstitial void volume. In some embodiments, the assay particle is capable of being magnetically manipulated via application of a magnetic force. In some embodiments, magnetic manipulation includes one or more of capture, isolation, washing, and/or movement within a fluid. In other embodiments, the assay particle does not comprise a magnetic element and/or is not capable of being magnetically manipulated via application of a magnetic force.

In some embodiments, the sample is an aqueous sample.

In some embodiments, the assay-particle-identifying physical characteristic comprises one or more of particle dimensions (e.g., size, volume, diameter, longest dimension, surface area, thickness, etc.), particle shape, particle morphology, emitted wavelength, color, signal strength, signal intensity, wavenumber, distribution or composition of magnetic particles within the assay particle, optical density, distribution of optical density of other optical features (e.g., evenly distributed, surface distribution, etc.), and a spectral signature. In some embodiments, the spectral signature is not excitable by the luminescent reporter. In some embodiments, the spectral signature is separately excitable from the luminescent reporter. In some embodiments, residual excitation of the spectral signature by the luminescent reporter is at least 10-fold less than (i.e., less than 1/10 of) direct excitation of the unique spectral signature (e.g., >20-fold less (< 1/20 of), >50-fold less (< 1/50 of), >100-fold less (< 1/100 of), >200-fold less (< 1/200 of), >500-fold less (< 1/500 of), >1,000-fold less (< 1/1,000 of), >2,000-fold less (< 1/2,000 of), >5,000-fold less (< 1/5,000 of), >10,000-fold less (< 1/10,000 of), etc.). In some embodiments, residual excitation of the spectral signature by the luminescent reporter is within background. In some embodiments, the spectral signature is excitable by the luminescent reporter. In some embodiments, the spectral signature is suitable for emitting a detectable signature signal. In some embodiments, the detectable signature signal is selected from a fluorescent signal, a colorimetric signal, an optical density-based signal, a Raman signal, an infrared signal, a chemiluminescent signal, and a bioluminescent signal. In some embodiments, the detectable signature signal can be correlated to binding of the target analyte to the target-specific interaction moiety. In some embodiments, the detectable spectral signature can be identified by one or more of signal wavelength, color, signal strength, signal intensity, and/or wavenumber.

In some embodiments, a composition comprises at least two distinct assay particles, wherein the at least two distinct assay particles comprise the same luminescent reporter, but a different target-specific interaction moiety(s) and different assay-particle-identifying physical characteristic(s) (e.g., size and/or spectral signatures). In some embodiments, a composition comprises a population of assay particles comprising two or more distinct subsets of assay particles; assay particles within the same subset comprise the same target-specific luminescent detection agent (same luminescent reporter and same target-specific interaction moiety) and the same assay-particle-identifying physical characteristic; assay particles within different subsets of the population comprise the same luminescent reporters but different target-specific interaction moieties and different assay-particle-identifying physical characteristics.

In a second aspect, methods are provided for conducting an assay using, for example, a particle described in the first aspect. In some embodiments, provided herein are methods of conducting an assay using an assay particle described in any embodiments herein, the method comprising: (a) contacting the assay particle with a sample and allowing the interstitial void volume of the assay particle to receive a portion of the sample; (b) allowing the target-specific interaction moiety of the assay particle to interact with a target analyte, if present, in the sample; and (c) detecting a luminescent signal produced from the luminescent reporter upon interaction of the target-specific interaction moiety with the target analyte.

In some embodiments, methods further comprise (d) detecting the assay-particle-identifying physical characteristic (e.g., spectral signature) of the assay particle. In some embodiments, methods further comprise identifying the assay particle based on the physical characteristic.

In some embodiments, the sample is selected from an environmental sample (e.g., wastewater (e.g., for detecting contaminants), soil (e.g., for detecting pesticides, heavy metals, or toxins), drinking water (e.g., for detecting contaminants such as heavy metals, bacteria, or pollutants), rainwater (e.g., for detecting pollutants or acid rain components), marine samples (e.g., for detecting oil spills, heavy metals, or microplastics), groundwater (e.g., for detecting chemicals, minerals, or pollutants), industrial samples, pharmaceutical samples (e.g., for detecting active pharmaceutical ingredients, impurities, or contaminants), food and beverage samples (e.g., for detecting additives, contaminants, or pathogens), chemical process samples (e.g., for detecting reaction by-products, purity levels, or specific chemicals), blood samples (e.g., for detecting hormones, pathogens, drugs, or metabolic products), urine samples (e.g., for detecting drugs, metabolic markers, or pathogens), breast milk, saliva samples (e.g., for detecting hormones, drugs, or DNA), semen samples (e.g., for detecting sperm count, motility, or infections), sweat samples (e.g., for detecting electrolytes or drugs), tissue samples (e.g., for detecting disease markers, toxins, or proteins), cell culture media (e.g., for detecting metabolites, growth factors, or contaminants), cell culture samples, cell lysates, sputum samples (e.g., for detecting respiratory pathogens or biomarkers), cerebrospinal fluid (CSF) (e.g., for detecting neurological diseases or infections), fecal samples (e.g., for detecting gut microbiome markers, pathogens, or toxins), viral or bacterial cultures (e.g., for detecting pathogens or assessing antimicrobial resistance), cancer tissue biopsy samples (e.g., for detecting tumor markers or genetic mutations), serum samples (e.g., for detecting biomarkers, drugs, or antibodies), plasma samples (e.g., for detecting glucose, lipids, or drug levels), agricultural samples (e.g., for detecting plant pathogens, pesticides, growth hormones, contaminants, etc.), forensic or toxicological samples (e.g., for detecting drugs, blood traces, DNA, poison, heavy metals, etc.), biochemical solutions (e.g., for detecting protein concentrations, enzymes, DNA/RNA sequences, etc.), RNA samples (e.g., for gene expression analysis or pathogen detection), DNA samples (e.g., for genetic analysis or pathogen identification), protein samples (e.g., for detecting specific proteins or enzyme activities), metabolite solutions (e.g., for detecting small molecules), patient specimens (e.g., urine, blood, tissue, etc.), microbiological culture samples (e.g., for detecting bacterial growth or antibiotic resistance profiles, etc.), etc.

In some embodiments, methods further comprise a step, prior to step (c), of confining a portion of the sample to an isolated reaction space within the assay particle by dispersing the assay particle in a non-aqueous medium, wherein the portion of the sample confined to the isolated reaction space cannot exit the isolated reaction space. In some embodiments, the non-aqueous medium comprises oil and an emulsifier. In some embodiments, aqueous liquid, and/or a water-soluble agent outside of the assay particle cannot enter the isolated reaction space without a suitable carrier. In some embodiments, a suitable carrier comprises oil and an emulsifier.

In some embodiments, methods further comprise a step, prior to step (c), of triggering an interaction of the target analyte with the target-specific interaction moiety. In some embodiments, triggering the interaction of the target analyte with the target-specific interaction moiety of the assay particle comprises cleaving a tether between the target-specific luminescent detection agent and the matrix material. In some embodiments, triggering the interaction of the target analyte with the target-specific interaction moiety of the assay particle comprises uncaging the target-specific interaction moiety.

In some embodiments, the interstitial void volume, and the sample outside of the assay particle are in fluid communication during steps (b) and/or (c). In some embodiments, the interstitial void volume is not converted into an isolated reaction space.

In some embodiments, methods further comprise a step, prior to step (c), of contacting the assay particle with a substrate for the luminescent reporter. In some embodiments, the substrate comprises an emulsified formulation in a non-aqueous medium. In some embodiments, the non-aqueous medium comprises an oil and an emulsifier.

In some embodiments, the sample is an aqueous sample.

In some embodiments, provided herein are methods of conducting an assay using a composition comprising the assay particles described herein, the method comprising: (a) contacting the assay particle with a sample and allowing the interstitial void volume to receive a portion of the sample; (b) confining a portion of the sample to an isolated reaction space, wherein the portion of the sample confined to the isolated reaction space cannot exit the isolated reaction space, and a sample outside the assay particle cannot enter the isolated reaction space; (c) allowing the target analyte, if present in the sample, to interact with the target-specific interaction moiety; (d) detecting the luminescent signal from the luminescent reporter; (e) detecting the unique assay-particle-identifying physical characteristic; (f) identifying the unique assay-particle-identifying physical characteristic; and (g) quantitating the interaction of the target analyte and the target-specific interaction moiety. In some embodiments, the sample is an aqueous sample. In some embodiments, confining a portion of the sample to an isolated reaction space comprises contacting the assay particle with a non-aqueous medium. In some embodiments, the non-aqueous medium comprises oil and an emulsifier. In some embodiments, aqueous liquid and/or a water-soluble agent outside of the assay particle cannot enter the isolated reaction space without a suitable carrier. In some embodiments, a suitable carrier comprises oil and an emulsifier.

In some embodiments, provided herein are methods of conducting an assay using the composition comprising assay particles herein (e.g., assay particles of the first aspect), the method comprising: (a) contacting the assay particle with a sample and allowing the interstitial void volume to receive a portion of the sample; (b) allowing the target analyte, if present in the sample, to interact with the target-specific interaction moiety; (c) detecting the luminescent signal from the luminescent reporter; (d) detecting the assay-particle-identifying physical characteristic; (e) identifying the assay-particle by the assay-particle-identifying physical characteristic; and (f) quantitating the interaction of the target analyte and the target-specific interaction moiety; wherein the interstitial void volume and a portion of the sample outside of the assay particle remain in fluid communication during steps (a) through (f). In some embodiments, the assay particle is not removed from the sample, and the assay particles are not washed between steps (a) and (f).

In a third aspect, compositions are provided comprising a plurality of distinct assay particles, such as any assay particles described in the first aspect. In some embodiments, provided herein are compositions comprising a plurality of distinct assay particles as described herein, wherein each of the distinct assay particles are porous, comprises a matrix material, and comprises an interstitial void volume suitable to receive a sample; wherein each of the distinct assay particles comprises a target-specific luminescent detection agent; wherein the target-specific luminescent detection agent comprises: (i) a unique target-specific interaction moiety, wherein the unique target-specific interaction moiety is different in each of the distinct assay particles, and (ii) a luminescent reporter suitable for producing a luminescent signal; wherein the luminescent reporter is the same in each of the distinct assay particles, and wherein interaction of the unique target-specific interaction moiety with the target analyte results in a change in the luminescent signal; wherein each of the distinct assay particles comprise a unique spectral signature.

In some embodiments, provided herein is a composition comprising a population of assay particles, wherein each of the assay particles is porous, comprises a matrix material, and comprises an interstitial void volume suitable to receive a sample; wherein the population of assay particles comprises two or more distinct subsets of assay particles; wherein each of the assay particles comprises a target-specific luminescent detection agent; wherein the target-specific luminescent detection agent of the assay particles of each subset comprises: (i) a target-specific interaction moiety that is unique to the subset, wherein the unique target-specific interaction moiety is different in each of the subsets, and (ii) a luminescent reporter suitable for producing a luminescent signal; wherein the luminescent reporter is the same in each of the subsets; wherein the assay particles comprise an assay-particle-identifying physical characteristic that is detectable and independent of the luminescent reporter; wherein the assay-particle-identifying physical characteristic of each of the subsets is unique to the assay particles of the subset; and wherein the assay particles of different subsets are distinguishable by the detectable physical characteristic of the assay particles.

In some embodiments, the assay particles are microparticles, beads, or microbeads. In some embodiments, the matrix material comprises any natural, synthetic, or hybrid material capable of forming a porous structure that can receive and contain a sample. In some embodiments, the matrix material comprises one or more biocompatible polymers. In some embodiments, the one or more biocompatible polymers are natural polymers selected from agarose, chitosan, alginate, chitin, gelatin, carrageenan, collagen, fucoidan, cellulose, laminaran, fibrin, hyaluronic acid, carboxymethyl cellulose, starch, pectin, keratin, chondroitin sulfate, pullulan, xanthan gum, arabic gum, ghatti gum, guar gum, locust bean gum, tragacanth gum, karaya gum, inulin, and albumin. In some embodiments, the one or more biocompatible polymers are synthetic polymers selected from polylactic acid (PLA), poly(lactic-co-glycolic acid) (PLGA), polycaprolactone (PCL), polyethylene glycol (PEG), polyvinyl alcohol (PVA), polymethyl methacrylate (PMMA), polyurethane (PU), poly(D,L-lactic acid) (PDLLA), polyethylene-co-vinyl acetate (EVA), poly(N-isopropylacrylamide) (PNIPAAm), polysiloxane (Silicone), poly(glycolic acid) (PGA), poly(ε-caprolactone-co-lactide) (PCLLA), poly(3-hydroxybutyrate) (PHB), polyvinylidene fluoride (PVDF), poly(alkylene oxides), and citric acid based polymers. In some embodiments, the one or more biocompatible polymers comprise natural and synthetic polymers. In some embodiments, the matrix material is liquefiable. In some embodiments, the matrix material is liquefied upon (1) application of heat, light, and/or enzyme, or (2) a change of pH, redox potential, ionic strength, temperature, magnetic field, or electromagnetic radiation. In some embodiments, the assay particles are prefabricated. In some embodiments, the assay particles are prefabricated by one or more of solvent casting, electrospinning, spray-drying, emulsion, mechanical grinding, phase separation, electrowriting, coacervation, spheronization, and lyophilizing. In some embodiments, the assay particles are lyophilized or dehydrated. In some embodiments, the assay particles are suitable for rehydration upon receiving a sample. In some embodiments, the assay particles form a porous hydrogel upon receiving a sample, and wherein the sample fills the interstitial void volume. In some embodiments, the assay particles are fully or partially hydrated. In some embodiments, the assay particles are suitable for rehydration upon receiving a sample.

In some embodiments, the sample fills the interstitial void volume within the assay particles when the assay particles receive the sample. In some embodiments, the interstitial void volume is suitable for receiving and containing molecules and/or macromolecules, which are present within the sample. In some embodiments, the interstitial void volume of each of the assay particles provides reaction spaces for conducting assays to detect one or more target analytes in a sample.

In some embodiments, the assay particles are dispersible in a non-aqueous medium. In some embodiments, upon dispersion in the non-aqueous medium, the interstitial void volumes provide isolated reaction spaces for conducting assays. In some embodiments, upon receipt of a first sample and thereupon dispersion in the non-aqueous medium, the isolated reaction spaces are suitable to confine a portion of the first sample within the isolated reaction spaces and prevent the entrance into the isolated reaction space of a second sample.

In some embodiments, the interstitial void volumes are in fluid communication with the exterior of the assay particles.

In some embodiments, the target-specific luminescent detection agent is contained within the interstitial void volume. In some embodiments, the target-specific luminescent detection agent is tethered to the matrix material. In some embodiments, the target-specific luminescent detection agent is covalently tethered to the matrix material. In some embodiments, the target-specific luminescent detection agent is irreversibly tethered to the matrix material. In some embodiments, the target-specific luminescent detection agent is reversibly tethered to the matrix material. In some embodiments, the target-specific luminescent detection agent is reversibly tethered to the matrix material by a cleavable linker. In some embodiments, the cleavable linker is light, chemical, heat, pH, or enzymatically cleavable. In some embodiments, target-specific luminescent detection agent is reversibly tethered to the matrix material by a noncovalent affinity interaction.

In some embodiments, the luminescent reporter is a bioluminescent reporter. In some embodiments, the bioluminescent reporter is a bioluminescent protein. In some embodiments, the bioluminescent protein is a luciferase. In some embodiments, luminescent reporter is a chemiluminescent reporter. In some embodiments, the chemiluminescent reporter comprises horseradish peroxidase, alkaline phosphatase, lactase, glucose oxidase, catalase, acid phosphatase, nitrate reductase, glutathione peroxidase, or choline oxidase. In some embodiments, the luminescent reporter is a fluorescent reporter. In some embodiments, the fluorescent reporter is a fluorescent protein. In some embodiments, the luminescent reporter is a luminescent complex comprising first and second components. In some embodiments, the first and second components are structurally complementary components of a bioluminescent or fluorescent complex. In some embodiments, the first and second components are a resonance energy transfer (RET) pair. In some embodiments, the first and second components are tethered together directly or by a linker.

In some embodiments, compositions (e.g., assay particles) further comprise a secondary detection agent, wherein the secondary detection agent comprises (i) a secondary reporter, and (ii) (A) a secondary target-specific interaction moiety or (B) a target mimetic. In some embodiments, the luminescent reporter and the secondary reporter comprise a BRET pair. In some embodiments, the luminescent reporter is a bioluminescent protein, and the secondary reporter is a fluorophore. In some embodiments, the bioluminescent protein is a luciferase. In some embodiments, the luminescent reporter and the secondary reporter comprise a FRET pair. In some embodiments, the luminescent reporter is a fluorescent protein, and the secondary reporter is a fluorophore. In some embodiments, the luminescent reporter and secondary reporter are first and second components of a complementation-dependent luminescent complex. In some embodiments, the secondary detection agent comprises a secondary target-specific interaction moiety, and wherein the target-specific interaction moiety and the secondary target-specific interaction moiety bind to separate locations on the target. In some embodiments, the secondary detection agent comprises a target mimetic, and wherein the target mimetic is suitable for binding to the target-specific interaction moiety. In some embodiments, the target mimetic is suitable for competing with the target for binding to the target-specific interaction moiety. In some embodiments, the secondary detection agent is within the interstitial void volume but not tethered to the matrix material. In some embodiments, the secondary detection agent is reversibly tethered to the matrix material. In some embodiments, the secondary detection agent is irreversibly tethered to the matrix material. In some embodiments, the secondary detection agent is tethered to the target-specific luminescent detection agent.

In some embodiments, the change in the luminescent signal is an increase in signal, a decrease in signal, generation of a signal, elimination of a signal, increase in resonance energy transfer, or decrease in resonance energy transfer.

In some embodiments, the target analyte is a small molecule, a peptide, a protein, a nucleic acid, a lipid, or an antibody. In some embodiments, the target-specific interaction moiety is a target-specific binding agent. In some embodiments, the target-specific binding agent is an antibody, an antibody fragment, a natural target binding domain, an affinity molecule, an affimer, an aptamer, a designed ankyrin repeat protein (DARPin), a ligand, or a protein. In some embodiments, the target-specific binding agent of each of the distinct assay particles of each binds or is suitable for binding to a distinct target analyte. In some embodiments, the target-specific binding agent of each of the distinct assay particles is suitable for binding to the same target analyte. In some embodiments, the unique target-specific interaction moiety comprises a ligand for the target analyte. In some embodiments, the target analyte comprises a ligand for the unique target-specific interaction moiety. In some embodiments, the assay-particle-identifying physical characteristic comprises one or more of particle dimensions (e.g., size, volume, diameter, longest dimension, surface area, thickness, etc.), particle shape, particle morphology, emitted wavelength, color, signal strength, signal intensity, wavenumber, distribution or composition of magnetic particles within the assay particle, optical density, distribution of optical density of other optical features (e.g., evenly distributed, surface distribution, etc.), and a spectral signature. In some embodiments, the assay-particle-identifying physical characteristic is a spectral signature selected from a fluorescent signal, a colorimetric signal, a Raman signal, an infrared signal, a chemiluminescent signal, and a bioluminescent signal. In some embodiments, the spectral signature is not excitable by the luminescent reporter. In some embodiments, the spectral signature is separately excitable from the luminescent reporter. In some embodiments, residual excitation of the spectral signature by the luminescent reporter is at least 10-fold (e.g., 20-fold, 50-fold, 100-fold, 200-fold-500-fold, 1000-fold) less than direct excitation of the spectral signature. In some embodiments, residual excitation of the spectral signature by the luminescent reporter is within background. In some embodiments, the spectral signature is excitable by the luminescent reporter. In some embodiments, the spectral signature is suitable for emitting a detectable signature signal.

In some embodiments, the assay-particle-identifying physical characteristic of each subset can be correlated to the identity of the target-specific interaction moiety of the assay particles of each subset. In some embodiments, the assay-particle-identifying physical characteristic of each of the distinct assay particles can be differentiated by one or more of particle dimensions (e.g., size, volume, diameter, longest dimension, surface area, thickness, etc.), particle shape, particle morphology, emitted wavelength, color, signal strength, signal intensity, wavenumber, distribution or composition of magnetic particles within the assay particle, optical density, distribution of optical density of other optical features (e.g., evenly distributed, surface distribution, etc.), and a spectral signature.

In a fourth aspect, methods of conducting multiplex assays are provided. In some embodiments, methods are provided for cross-reactivity-free (or substantially cross-reactivity-free) multiplex assays are provided using, for example, the assay particles of the first aspect, methods of the second aspect, and/or the plurality of distinct assay particles of the third aspect. In some embodiments, provided herein are methods of conducting cross-reactivity-free (or substantially cross-reactivity-free) multiplex assays comprising: (a) contacting a composition comprising a plurality of distinct assay particles described herein with a sample and allowing the interstitial void volume of each of the distinct assay particles to receive a portion of the sample; (b) allowing the unique target-specific interaction moiety of the distinct assay particles to interact with a target analyte, if present, in the sample; (c) detecting the luminescent signal from the luminescent reporter in each of the distinct assay particles; and (d) detecting the assay-particle-identifying physical characteristic from each of the distinct assay particles.

In some embodiments, methods further comprise identifying the unique assay-particle-identifying physical characteristic from each of the distinct assay particles, thereby identifying each of the distinct assay particles. In some embodiments, methods further comprise correlating the luminescent signal from the luminescent reporter to interaction of the target analyte and target-specific interaction moiety based on the identification of the assay particles.

In some embodiments, methods further comprise a step, prior to step (c), of confining a portion of the sample to isolated reaction spaces within the distinct assay particles, wherein the portion of the sample confined to the isolated reaction spaces of each of the distinct assay particles cannot move between the distinct assay particles or exit the distinct assay particles. In some embodiments, the sample outside of the distinct assay particles cannot enter the isolated reaction space without a suitable carrier.

In some embodiments, methods further comprise a step, prior to step (c), of confining portions of the sample to an isolated reaction space within each of the distinct assay particles by dispersing the distinct assay particles in a non-aqueous medium, wherein the portion of the sample confined to the isolated reaction spaces cannot exit the isolated reaction spaces. In some embodiments, the non-aqueous medium comprises an oil and an emulsifier. In some embodiments, aqueous liquid and/or a water-soluble agent outside of the assay particle cannot enter the isolated reaction space without a suitable carrier. In some embodiments, a suitable carrier comprises oil and an emulsifier.

In some embodiments, the interstitial void volume and the sample outside of the assay particles are in fluid communication during steps (b), (c), and/or (d). In some embodiments, methods comprise a step of maintaining fluid communication between the interstitial void volume and the sample outside of the assay particles. In some embodiments, the interstitial void volume is not converted into an isolated reaction space.

In some embodiments, methods further comprise a step, prior to step (c), of triggering an interaction of the target analyte with the unique target-specific interaction moiety of the distinct assay particles if the target analyte is present or not triggering an interaction of the target analyte with the unique target-specific interaction moiety of the distinct assay particles if the target analyte is not present. In some embodiments, triggering an interaction of the target analyte with the unique target-specific interaction moiety of the distinct assay particles comprises cleaving a tether between the target-specific luminescent detection agent and the matrix material.

In some embodiments, methods further comprise a step, prior to step (c), of contacting the distinct assay particles with a substrate for the luminescent reporter of the distinct assay particles. In some embodiments, the substrate is an emulsified formulation in a non-aqueous medium. In some embodiments, the non-aqueous medium comprises an oil and an emulsifier.

In some embodiments, provided herein are methods of conducting cross-reactivity-free (or substantially cross-reactivity-free) multiplex assays in isolated reaction spaces within a plurality of assay particles comprising: (a) contacting the composition comprising a population of assay particles described herein with a sample and allowing the interstitial void volume of each of the distinct assay particles to receive a portion of the sample; (b) confining a portion of the sample to an isolated reaction space within each of the distinct assay particles, wherein (i) the portion of the sample confined to the isolated reaction space of each of the distinct assay particles cannot move between the distinct assay particles or exit the distinct assay particles; and (ii) a component outside of the distinct assay particles cannot enter the isolated reaction space of the distinct assay particles without a suitable carrier; (c) allowing the unique target-specific interaction moiety of each of the distinct assay particles to interact with a target analyte, if present, in the sample; (d) detecting the luminescent signal from the luminescent reporter in each of the distinct assay particles; (e) detecting the assay-particle-identifying physical characteristic from the assay particles; (f) quantitating the interaction of the target analyte and the unique target-specific interaction moiety; and (g) identifying the unique spectral signature from each of the distinct assay particles, thereby identifying each of the distinct assay particles. In some embodiments, further comprising a step prior to step (c) of triggering interaction of the target analyte with the unique target-specific interaction moiety of the distinct assay particles. In some embodiments, further comprising a step before step (d) of contacting the distinct assay particles with a substrate for the luminescent reporter of the distinct assay particles. In some embodiments, methods herein further comprise one or more washing steps. In some embodiments, methods do not comprise washing steps.

In some embodiments, provided herein are methods of conducting cross-reactivity-free (or substantially cross-reactivity-free) multiplex assays a plurality of assay particles that remain in fluid communication with the same sample, the methods comprising: (a) contacting a composition comprising a plurality of assay particles (e.g., a population of the third aspect) with a sample and allowing the interstitial void volume of the assay particles to receive a portion of the sample; (b) allowing the target-specific interaction moiety of each of the distinct assay particles to interact with a target analyte, if present, in the sample; (c) detecting the luminescent signal from the luminescent reporter in each of the distinct assay particles; (d) detecting the assay-particle-identifying physical characteristic from the assay particles; (e) quantitating the interaction of the target analyte and the unique target-specific interaction moiety; and (f) identifying the assay-particle-identifying physical characteristic from each of the distinct assay particles, thereby identifying each of the distinct assay particles. In some embodiments, methods further comprise a step before step (c) of contacting the distinct assay particles with a substrate for the luminescent reporter of the distinct assay particles. In some embodiments, methods further comprise contacting the distinct assay particles with a substrate for the luminescent reporter of the distinct assay particles. In some embodiments, methods are conducted without washing the assay particle between step (a) and (f).

In a fifth aspect, methods are provided for conducting assays using, for example, the assay particles of the first aspect, the methods of the second aspect, the plurality of distinct assay particles of the third aspect, and/or steps/methods of the fourth aspect. In some embodiments, provided herein are assay methods comprising: (a) contacting (i) comprising a population of assay particles comprising at least two different subsets of distinct assay particles, wherein the assay particles within a subset comprise the same target-specific luminescent detection agent and the same assay-particle-identifying physical characteristic, but the assay particles in different subsets comprise different target-specific luminescent detection agents and different assay-particle-identifying physical characteristic with (ii) a sample and allowing the interstitial void volumes of assay particles of the at least two different subsets to receive a portion of the sample; (b) allowing the unique target-specific interaction moiety of a assay particles of the at least two different subsets to interact with a target analyte for the unique target-specific moieties, if present, in the sample; (c) detecting the luminescent signal from the luminescent reporter in the assay particles of the at least two different subsets; (d) detecting the assay-particle-identifying physical characteristic from the assay particles of the at least two different subsets; (e) identifying the assay-particle-identifying physical characteristic from the assay particles of the at least two different subsets, thereby identifying each of the distinct assay particles; and (f) quantifying the interaction between the target analyte with each unique target-specific interaction moiety based on the proportion of at least two of the distinct assay particles that exhibit a change in luminescence.

In some embodiments, methods further comprise a step of confining the portion of the sample to an isolated reaction space within at least two of the distinct assay particles.

In some embodiments, the interstitial void volume and a portion of the sample outside of the assay particles remain in fluid communication during steps (b) through (f). In some embodiments, the assay particles are not removed from the sample, and the assay particles are not washed between steps (b) and (f). In some embodiments, methods comprise maintaining fluid communication between the interstitial void volumes and the sample outside of the assay particles.

In some embodiments, the sample is an aqueous sample.

In a sixth aspect, compositions for conducting multiplex sandwich assays (e.g., for the detection of multiple different analytes in a sample) using, for example, the assay particles of the first aspect, the methods of the second aspect, and/or the plurality of distinct assay particles of the third aspect. In some embodiments, provided herein are compositions for conducting multiplex sandwich assays for the detection of multiple different analytes in a single sample, the compositions comprising a plurality of distinct assay particles, each configured for the detection of a target analyte specific to the distinct assay particle; wherein each of the distinct assay particles is porous, comprises a matrix material, and comprises an interstitial void volume suitable to receive the sample; wherein each of the distinct assay particles comprises a first target-specific luminescent detection agent; wherein the first target-specific luminescent detection agent comprises: (i) a first target-specific binding agent configured to bind to a first portion of the target analyte specific to the distinct assay particle, wherein the first target-specific binding agent is different in each of the distinct assay particles and is configured to bind to a different target analyte, and (ii) a first component of a luminescent reporter; wherein the first component of the luminescent reporter is the same in each of the distinct assay particles; wherein each of the distinct assay particles comprises a second target-specific luminescent detection agent; wherein the second target-specific luminescent detection agent comprises: (i) a second target-specific binding agent configured to bind to a second portion of the target analyte, and (ii) a second component of the luminescent reporter; wherein the second component of the luminescent reporter is the same in each of the distinct assay particles; wherein, upon the binding of the first and second target-specific binding agents to the target analyte, the first and second components of the luminescent reporter interact to form an active luminescent reporter; and wherein each of the distinct assay particles comprise a unique assay-particle-identifying physical characteristic that is detectable and independent of the luminescent reporter; wherein the distinct assay particles are distinguishable by the detectable physical characteristic of the assay particles.

In some embodiments, the second target-specific binding agent is different in each of the distinct assay particles and is configured to bind to a different target analyte. In some embodiments, the second target-specific binding agent is the same in each of the distinct assay particles and is configured to bind to each of the target analytes of the distinct assay particles. In some embodiments, the first and/or second target-specific binding agents are tethered to the matrix material.

In some embodiments, the distinct assay particles are dispersible in a non-aqueous medium and, upon dispersion in the non-aqueous medium, the distinct assay particles provide an isolated reaction space for detection of the target analyte. In some embodiments, the distinct assay particles are dispersible in an aqueous medium. Some embodiments herein comprise a step of dispersing the distinct assay particles in a non-aqueous or aqueous medium.

In some embodiments, the luminescent reporter is a bioluminescent reporter. In some embodiments, the bioluminescent reporter comprises a complementation-dependent bioluminescent complex. In some embodiments, the first and second components of the luminescent reporter are a peptide and a polypeptide that interact through structural complementation to form the complementation-dependent bioluminescent complex (e.g., NANOBIT). In some embodiments, the first and second components of the luminescent reporter are first and second peptides that interact through structural complementation with a third polypeptide component to form the complementation-dependent bioluminescent complex (e.g., NANOTRIP). In some embodiments, the first and the second components of the luminescent reporter are a luciferase and a fluorophore that form a bioluminescence resonance energy transfer (BRET) pair. In some embodiments, the first component of the luminescent reporter is a bioluminescent protein or complex (e.g., of two or more sub-components), and the second component is a fluorophore capable of being excited by the first component. In some embodiments, the first component of the luminescent reporter is a donor fluorophore, and the second component is an acceptor fluorophore capable of being excited by the first component.

In some embodiments, the first target-specific luminescent detection agent and/or the second target-specific luminescent detection agent are linked to the matrix material by a reversible tether. In some embodiments, breaking of the reversible tether allows both the first target-specific binding agent and the second target-specific binding agent to bind the target analyte, if present, in the interstitial void volume of the distinct assay particles.

In some embodiments, the first and the second target-specific binding agents independently are an antibody, an antibody fragment, a natural target binding domain, an affinity molecule, a computationally derived binding domain, an affimer, an aptamer, a designed ankyrin repeat protein (DARPin), a ligand, or a protein.

In some embodiments, the first target-specific binding agent is different in each of the distinct assay particles. In other embodiments, the first target-specific binding agent is identical in each of the distinct assay particles.

In some embodiments, the second target-specific binding agent is different in each of the distinct assay particles. In other embodiments, the second target-specific binding agent is identical in each of the distinct assay particles.

In some embodiments, at least one of the first and second target-specific binding agents are different in each of the distinct assay particles. In some embodiments, the first and second target-specific binding agents are both different in each of the distinct assay particles. In some embodiments, the distinct assay particles are dispersed in a non-aqueous medium. In some embodiments, the interstitial void volumes of the distinct assay particles are in fluid communication with the sample. In some embodiments, the sample is an aqueous sample. In some embodiments, the distinct assay particles are lyophilized or dehydrated.

In a seventh aspect, methods of conducting multiplex sandwich assays to detect a target analyte are provided, for example, using the assay particles of the first aspect, the methods of the second aspect, and/or plurality of distinct assay particles of the third aspect. In some embodiments, provided herein are methods of conducting multiplex sandwich assays to detect a multiple different target analytes, the methods comprising: (a) contacting a plurality of distinct assay particles (e.g., subsets of the plurality) described herein (e.g., wherein each distinct assay particle or subset of assay particles comprises a different target-specific binding agent and a unique assay-particle-identifying physical characteristic) with a sample and allowing the interstitial void volume of each of the distinct assay particles to receive a portion of the sample; (b) allowing the multiple different target analytes, if present, to interact with the first and the second target-specific binding agents of the distinct assay particles; (c) detecting the luminescent signal from the luminescent reporter in each of the distinct assay particles; and (d) detecting the unique assay-particle-identifying physical characteristic from each of the distinct assay particles.

In some embodiments, methods further comprise confining a portion of the sample to isolated reaction spaces within each of the distinct assay particles, wherein (i) the portion of the sample confined to the isolated reaction spaces of each of the distinct assay particles cannot move between distinct assay particles or exit the distinct assay particles; and (ii) a component outside the distinct assay particles cannot enter the isolated reaction spaces without a suitable carrier.

In some embodiments, methods further comprise maintaining fluid communication between the interstitial void volumes and the sample outside of the assay particles during steps (b) through (d).

In some embodiments, methods further comprise a step, after step (a), of cleaving the tethers between the matrix material and the first and second target-specific luminescent detection agents. In some embodiments, methods further comprise a step, after step (a) but before step (b), of cleaving the tethers between the matrix material and the first and second target-specific luminescent detection agents. In some embodiments, methods further comprise a step, after step (b), of cleaving the tethers between the matrix material and the first and second target-specific luminescent detection agents. In some embodiments, prior to cleavage of the tethers, a target analyte can bind to either the first or second target-specific luminescent detection agents, but not both. In some embodiments, after cleavage of the tethers, a target analyte can bind to both the first and second target-specific luminescent detection agents. In some embodiments, methods further comprise contacting the distinct assay particles with a substrate for the luminescent reporter. In some embodiments, methods further comprise quantitating the interaction of the target analyte and the target-specific binding agents based on the amount of luminescent signal. In some embodiments, methods further comprise identifying each of the distinct assay particles by the unique assay-particle-identifying physical characteristic.

In an eight aspect, sandwich assay methods are provided, for example, using the assay particles of the first aspect, the methods of the second aspect, plurality of distinct assay particles of the third aspect, and/or methods of conducting multiplex sandwich assays of the seventh aspect. In some embodiments, provided herein are sandwich assay methods comprising: (a) contacting a sample with a composition for conducting multiplex sandwich assays described herein (e.g., comprising a population of multiple subsets of distinct assay particles); (b) allowing the target analyte, if present within the sample portions of at least two distinct assay particles, to interact with the first and the second target-specific binding agents in at least two distinct assay particles; (c) contacting at least two distinct assay particles with a substrate for the luminescent reporter; (d) detecting the luminescent signal from the luminescent reporter in at least two distinct assay particles; (e) detecting the assay-particle-identifying physical characteristic from at least two distinct assay particles; (f) identifying the assay-particle-identifying physical characteristic from at least two distinct assay particles, thereby identifying each of the distinct assay particles; and (g) quantifying the interaction between the target analyte with the first and the second target-specific binding agents of each of the distinct assay particles based on the proportion of the multiple of each distinct assay particles that exhibit a change in luminescence.

In a ninth aspect, compositions for conducting multiplex target engagement assays are provided (e.g., to detect target engagement of multiple different target analytes, for example, using the assay particles of the first aspect, the methods of the second aspect, and/or the plurality of distinct assay particles of the third aspect. In some embodiments, provided herein are compositions for conducting multiplex target engagement assays, the compositions comprising a plurality of distinct assay particles, wherein each of the distinct assay particles is porous, comprises a matrix material, and comprises an interstitial void volume suitable to receive a sample; wherein each of the distinct assay particles contain a target engagement complex tethered to the matrix material; wherein the target engagement complex comprises: (a) (i) a target, wherein the target is different in each of the distinct assay particles, tethered to (ii) a first component of a luminescent reporter, wherein the first component of the luminescent reporter is the same in each of the distinct assay particles, and (b) a tracer comprising: (i) a known interaction moiety capable of binding non-covalently to the target of each of the distinct assay particles, and (ii) a second component of the luminescent reporter; wherein, when the known interaction moiety of the tracer is non-covalently bound to the target, the luminescent reporter is suitable to emit a detectable signal; wherein each of the distinct assay particles comprise a unique assay-particle-identifying physical characteristic.

In some embodiments, each of the distinct assay particles contain a target engagement complex tethered to the matrix material; wherein the target engagement complex comprises: (a) (i) a target, wherein the target is different in each of the distinct assay particles, tethered to (ii) a first component of a luminescent reporter, wherein the first component of the luminescent reporter is the same in each of the distinct assay particles, and (b) a tracer comprising: (i) a known interaction moiety capable of binding non-covalently to the target of each of the distinct assay particles, and (ii) a second component of the luminescent reporter; wherein, when the known interaction moiety of the tracer is non-covalently bound to the target, the luminescent reporter is suitable to emit a detectable signal; wherein each of the distinct assay particles comprise a unique assay-particle-identifying physical characteristic.

In some embodiments, the first and second components of the luminescent reporter are a luciferase and a fluorophore that form a bioluminescence resonance energy transfer (BRET) pair. In some embodiments, the luminescent reporter is a bioluminescent reporter. In some embodiments, the bioluminescent reporter comprises a complementation-dependent bioluminescent complex. In some embodiments, the first and second components of the complementation-dependent bioluminescent complex are a peptide and a polypeptide that interact through structural complementation to form the bioluminescent complex. In some embodiments, the first and second components of the complementation-dependent bioluminescent complex do not stably interact to form the bioluminescent complex when the interaction moiety of the tracer is not bound to the target.

In some embodiments, the target is a target protein. In some embodiments, the target protein is a different variant of the target of each of the distinct assay particles. In some embodiments, the target is a unique kinase, BET bromodomain-containing protein, cytoplasmic signaling protein (such as FKBP12), nucleoprotein, histone deacetylase, lysine methyl transferase, protein regulating angiogenesis, protein regulating immune response, aromatic hydrocarbon receptor (AHR), estrogen receptor, androgen receptor, glucocorticoid receptor, or transcription factor (e.g., SMARCA4, SMARCA2, TRIM24).

In some embodiments, the distinct assay particles are lyophilized or dehydrated.

In a tenth aspect, methods of conducting a multiplex target engagement assay using, for example, the compositions of the ninth aspect. In some embodiments, provided herein are methods of conducting a multiplex target engagement assay to detect a binding to a target, the methods comprising: (a) contacting a sample with a composition for conducting multiplex target engagement assays described herein (e.g., comprising a plurality of distinct assay particles); (b) allowing the interstitial void volume of each of the distinct assay particles to receive a portion of the sample; (c) allowing the interaction moiety of the tracer to bind to the target; (d) detecting the detectable signal from the luminescent reporter at a first time point; (e) contacting the distinct assay particles with a test interaction moiety; (f) allowing the interaction moiety of the test interaction moiety to bind to the target; (g) detecting the detectable signal from the luminescent reporter at a second time point; wherein a reduction in the detectable signal within a distinct assay particle at the second time point compared to the first time point indicates a binding of the test interaction moiety to the target; (h) detecting the unique assay-particle-identifying physical characteristic from each of the distinct assay particles; and (i) correlating the unique assay-particle-identifying physical characteristic to the detectable signal.

In an eleventh aspect, compositions comprising a plurality of distinct assay particles for use as an intramolecular sensor, for example, using the assay particles of the first aspect, the methods of the second aspect, and/or the plurality of distinct assay particles of the third aspect. In some embodiments, provided herein are compositions comprising a plurality of distinct assay particles, wherein each of the distinct assay particles is porous, comprises a matrix material, and comprises an interstitial void volume suitable to receive a sample; wherein each of the distinct assay particles comprise a target-specific luminescent detection agent tethered to the matrix material; wherein the target-specific luminescent detection agent comprises: (i) a first component of a luminescent reporter, (ii) a first unique target-specific binding agent, (iii) a second unique target-specific binding agent, and (iv) a second component of a luminescent reporter; wherein the first and the second unique target-specific binding agents are different in each of the distinct assay particles; wherein the first and the second components of the luminescent reporter are the same in each of the distinct assay particles; wherein interaction of the first and the second components of the luminescent reporter produces an active luminescent reporter suitable for emitting a luminescent signal; wherein the binding of the first and the second unique target-specific binding agents to the target analyte results in a change in the luminescent signal; wherein each of the distinct assay particles comprise a unique assay-particle-identifying physical characteristic; and wherein the unique assay-particle-identifying physical characteristic is not excitable by the luminescent reporter. In other embodiments, provided herein are compositions comprising a plurality of distinct assay particles, wherein each of the distinct assay particles is porous, comprises a matrix material, and comprises an interstitial void volume suitable to receive a sample; wherein each of the distinct assay particles comprise a target-specific luminescent detection agent tethered to the matrix material; wherein the target-specific luminescent detection agent comprises: (i) a first component of a luminescent reporter, (ii) a unique target-specific binding agent, and (iii) a second component of a luminescent reporter; wherein the unique target-specific binding agents are different in each of the distinct assay particles; wherein the first and the second components of the luminescent reporter are the same in each of the distinct assay particles; wherein the unique target-specific binding agent undergoes a conformational change upon binding to the target analyte such that upon binding the analyte, the first and the second components of the luminescent reporter are brought into proximity to produce a detectable luminescent signal; wherein each of the distinct assay particles comprise a unique assay-particle-identifying physical characteristic; and optionally wherein the unique assay-particle-identifying physical characteristic is not excitable by the luminescent reporter.

In some embodiments, the luminescent reporter is a bioluminescent reporter. In some embodiments, the bioluminescent reporter comprises a bioluminescence resonance energy transfer (BRET) pair. In some embodiments, the BRET pair comprises a bioluminescent protein and a fluorophore. In some embodiments, the bioluminescent protein is a luciferase. In some embodiments, the bioluminescent reporter comprises a complementation-dependent bioluminescent complex.

In some embodiments, the target analyte is a small molecule, a peptide, a protein, a nucleic acid, a lipid, or an antibody. In some embodiments, the target-specific binding agent is an antibody, an antibody fragment, a natural target binding domain, a computationally derived binding domain, an affinity molecule, an affimer, an aptamer, a designed ankyrin repeat protein (DARPin), a ligand, or a protein.

In some embodiments, the target-specific binding agent of each of the distinct assay particles is suitable for binding to a distinct target analyte. In some embodiments, the target-specific binding agent of each of the distinct assay particles is suitable for binding to the same target analyte.

In some embodiments, the first component of a luminescent reporter is linked to the second component of a luminescent reporter via the first unique target-specific binding agent and the second unique target-specific binding agent.

In a twelfth aspect, methods of conducting an intramolecular sensor assay are provided, for example, using the compositions of the eleventh aspect. In some embodiments, provided herein are methods of conducting an intramolecular sensor assay comprising: (a) contacting a composition a sample with a composition for conducting an intramolecular sensor assay (e.g., an assay particle comprising a target-specific luminescent detection agent comprising: (i) a first component of a luminescent reporter, (ii) a first unique target-specific binding agent, (iii) a second unique target-specific binding agent, and (iv) a second component of a luminescent reporter); (b) allowing the interstitial void volume of each of the distinct assay particles to receive a portion of the sample; (c) allowing the first and the second unique target-specific binding agents of the distinct assay particles to interact with a target analyte, if present, in the sample; (d) detecting the luminescent signal from the luminescent reporter in each of the distinct assay particles; and (e) detecting the unique assay-particle-identifying physical characteristic from each of the distinct assay particles.

In a thirteenth aspect, compositions comprising a plurality of distinct assay particles for conducting a cleavage assay are provided, for example, using the assay particles of the first aspect, the methods of the second aspect, and/or the plurality of distinct assay particles of the third aspect. In some embodiments, provided herein are compositions comprising a plurality of distinct assay particles, wherein each of the distinct assay particles is porous, comprises a matrix material, and comprises an interstitial void volume suitable to receive a sample; wherein each of the distinct assay particles comprise a target-specific luminescent detection agent tethered to the matrix material; wherein the target-specific luminescent detection agent comprises: a first component of a luminescent reporter linked to a second component of the luminescent reporter by a unique target-analyte-sensitive linker; wherein the unique target-analyte-sensitive linker is different in each of the distinct assay particles; wherein the first and the second components of the luminescent reporter are the same in each of the distinct assay particles; wherein interaction of the first and the second components of the luminescent reporter produces an active luminescent reporter suitable for emitting a luminescent signal; wherein cleavage of the unique target-analyte-sensitive linker disrupts the interaction of the first and the second components of the luminescent reporter; wherein each of the distinct assay particles comprise a u unique assay-particle-identifying physical characteristic; and optionally wherein the unique assay-particle-identifying physical characteristic is not excitable by the luminescent reporter.

In some embodiments, the luminescent reporter is a bioluminescent reporter. In some embodiments, the bioluminescent reporter comprises a bioluminescence resonance energy transfer (BRET) pair. In some embodiments, the BRET pair comprises a bioluminescent protein and a fluorophore. In some embodiments, the bioluminescent protein is a luciferase. In some embodiments, the bioluminescent reporter comprises a complementation-dependent bioluminescent complex.

In some embodiments, the unique target-analyte-sensitive linker of each distinct assay particle is sensitive to a different target analyte. In some embodiments, the unique target-analyte-sensitive linker is a protease-sensitive linker.

In a fourteenth aspect, methods of conducting cleavage assays are provided, for example, using the compositions of the thirteenth aspect. In some embodiments, provided herein are methods of conducting cleavage assays comprising: (a) contacting a sample with a composition for conducting protease assays (e.g., plurality of assay particles comprising a first component of a luminescent reporter linked to a second component of the luminescent reporter by a unique target-analyte-sensitive linker); (b) allowing the interstitial void volume of each of the distinct assay particles to receive a portion of the sample; (c) allowing the unique target-analyte-sensitive linker of each distinct assay particle to interact with a target analyte, if present, in the sample; (d) detecting the luminescent signal from the luminescent reporter in each of the distinct assay particles; and (e) detecting the unique assay-particle-identifying physical characteristic from each of the distinct assay particles.

In some embodiments, provided herein are compositions comprising a population of assay particles; wherein the population of assay particles comprises distinct subsets of assay particles; wherein each subset is specific for a different target analyte; wherein each assay particle comprises a detectable-identical luminescent reporter tethered to the assay particle, and exhibits a change in luminescent signal from the luminescent reporter in response to the target analyte of the subset; wherein the assay particles of each subset comprise a unique assay-particle-identifying physical characteristic. In some embodiments, provided herein are assay methods comprising: (a) exposing the composition to a sample; (b) detecting the luminescent signal from each assay particle of the population; (c) assigning each assay particle to a subset according to the assay-particle-identifying physical characteristic of the assay particle; and (d) correlating the luminescent signal for each assay particle to the analyte for the subset.

In some embodiments, any of the methods herein may comprise end-point detection and/or real-time detection.

The various aspects and embodiments above do not limit the full scope of the present technology. Additional details of the technology are provided in the DETAILED DESCRIPTION and EXPERIMENTAL sections. Further, any combinations of elements or steps of the compositions and methods of the various aspects and embodiments above may be combined within the scope herein.

### BRIEF DESCRIPTION OF THE DRAWINGS

FIG. 1. Cartoon depiction of exemplary assay particle with bioluminescence-based reporters tethered thereto. The assay particle provides a porous support for loading bioluminescence-dependent biosensors. The conjugation chemistry utilized to tether the biosensor is present on solvent-accessible surface areas assay particles, i.e., externally exposed surfaces as well as internal surfaces within the interstitial void volume (e.g., pores within the assay particle). Chemical tethering limits the bioluminescent enzymatic reaction to the confines of each assay particle. In this exemplary, particles a spectral signature is provided by encoded particles and manipulation of the assay particles in provided by magnetic particles within the assay particle.

FIG. 2. Scanned electron micrograph of freeze fractured exemplary assay particle revealing porous nature of the matrix material and interstitial void volume.

FIG. 3. Cartoon depiction of image-based analysis of luminescent assay signal (left) and spectral signature signal from a population of assay particles with multiple distinct subsets to allow for spatial multiplexing.

FIG. 4A-C. Intramolecular analyte detection. (A) Cartoon depiction of an exemplary generic intramolecular analyte detection assay that can be conducted within an assay particle herein. A target-specific luminescent detection agent is tethered to a secondary detection agent and conjugated to the matrix material of an assay particle; binding of the target analyte to both the target-specific interaction element and the secondary binding element is required to bring the luminescent reporter and secondary reporter into proximity to allow resonance energy transfer. (B) Cartoon depiction of an exemplary intramolecular Ca²⁺ detection assay that can be conducted within an assay particle herein. A HaloTag® protein is tethered to a NanoLuc® reporter by a pair of Ca²⁺ binding moieties, and NanoLuc is conjugated to an agarose matrix material; a luminophore substrate for NanoLuc and a fluorescent HaloTag® ligand are introduced into the assay particle; in the presence of Ca²⁺, binding of Ca²⁺ by both the Ca²⁺ binding moieties brings the NanoLuc® reporter and fluorescent HaloTag® ligand into proximity to allow resonance energy transfer. (C) Cartoon depiction of an exemplary generic intramolecular analyte detection assay that can be conducted within an assay particle herein. A target-specific luminescent detection agent comprising a single target-specific interaction moiety, a luminescent reporter, and a secondary reporter is conjugated to the matrix material of an assay particle; binding of the target analyte to target-specific interaction moiety results in a conformational change to the target-specific interaction moiety and brings the luminescent reporter and secondary reporter into proximity to allow resonance energy transfer.

FIG. 5A-B. Intermolecular analyte detection. (A) Cartoon depiction of an exemplary intermolecular analyte detection assay that can be conducted within an assay particle herein. A target-specific luminescent detection agent and a secondary detection agent are separately conjugated to the matrix material of an assay particle; binding of the target analyte to both the target-specific interaction element and the secondary binding element is required to bring the luminescent reporter and secondary reporter into proximity to allow resonance energy transfer. (B) Cartoon depiction of an exemplary intermolecular rapamycin detection assay that can be conducted within an assay particle herein. A HaloTag® protein is tethered to FRB, which is conjugated to an agarose matrix material; a NanoLuc® reporter is tethered to FKBP, which is conjugated to the agarose matrix material; a luminophore substrate for NanoLuc and a fluorescent HaloTag® ligand are introduced into the assay particle; in the presence of rapamycin, binding of formation of the FRP-rapamycin-FKBP ternary complex brings the NanoLuc® reporter and fluorescent HaloTag® ligand into proximity to allow resonance energy transfer.

FIG. 6. Cartoon depiction of an exemplary target engagement assay that can be conducted within an assay particle herein. A target-specific luminescent detection agent is conjugated to the matrix material of an assay particle (left). Upon binding of a secondary detection agent comprising a fluorophore linked to a target analyte mimetic to the target-specific interaction moiety, the fluorophore and the luminescent reporter are positioned within sufficient proximity to allow resonance energy transfer (middle). Addition of a sample comprising the target analyze to the system results in competition for binding to the target-specific interaction moiety and a reduction in resonance energy transfer (right).

FIG. 7. Cartoon depiction of an exemplary protease sensor assay that can be conducted within an assay particle herein. A luminescent donor linked via a protease-sensitive linker to a luminescent acceptor is conjugated to the matrix material of an assay particle, placing the donor and acceptor within sufficient proximity to allow resonance energy transfer (left). Addition of a sample comprising an appropriate protease to the system results in cleavage of the linker and release of the luminescent acceptor, thereby resulting in a reduction in resonance energy transfer (right).

FIG. 8A-B. Cartoon depictions of exemplary metabolite sensor assays that can be conducted within an assay particle herein. (A) Two-component metabolite sensor assay—luminescent reporter linked to a reductase enzyme is conjugated to the matrix material; a metabolite-specific dehydrogenase is also conjugated to the matrix material. (B) One-component metabolite sensor assay—a metabolite-specific dehydrogenase, luminescent reporter, and reductase enzyme are linked together (in any suitable arrangement) and are conjugated to the matrix material; a metabolite-specific dehydrogenase is also conjugated to the matrix material. In both (A) and (B), the presence of a specific metabolite, the metabolite-specific dehydrogenase converts NAD into NADH; in the presence of NADH, the reductase converts a pro-luminophore into a luminophore; and in the presence of the luminophore, the luminescent reporter emits a luminescent signal; therefore, the luminescent signal is dependent upon the presence of the metabolite.

FIG. 9. Cartoon depiction of an exemplary sandwich analyte sensor assay that can be conducted within an assay particle herein. Secondary antibodies linked to LgBiT and SmBiT components of the NanoBiT® bioluminescent structural complementation system are reversible conjugated to the matrix material; upon addition of a sample comprising an analyte, primary antibodies for the analyte that are capable of being bound by the secondary antibodies, and upon releasing the secondary antibodies from the matrix material, a complex is formed around the analyte capable of emitting luminescence in the presence of a luminophore substrate.

FIG. 10. Exemplary multiplex antibody detection assay. A sample comprising unknown antibodies is provided; a luminescently-labeled generic antibody binding agent (e.g., protein G) is added to non-specifically labeled antibodies in the sample; the sample is added to a population of assay particles comprising subsets of distinct assay particles, each subset comprising a known antigen and a unique spectral signature; correlating the spectral signature of a subset of assay particles to a luminescent signal in that subset identifies the presence of an antibody for the antigen of that subset in the sample.

FIG. 11A-D. Porous agarose assay particles loaded with bioluminescent reporters generate a stable bioluminescent signal. (A) Streptavidin-functionalized agarose assay particles were loaded with a HaloTag-NanoLuc fusion covalently labeled with a chloroalkane biotin ligand. The biotin-streptavidin interaction tethers the fusion protein to the beads. (B) Relative luminescence signal from 4000 streptavidin assay particles incubated with increasing amounts of the biotin-HaloTag-NanoLuc moiety. (C) Similar experiment to (B) but measurements taken at 5 minutes and 30 minutes. (D) Relative luminescence signal from increasing amounts Biotin-HaloTag-NanoLuc in free solution taken at 5 minutes and 30 minutes, in the absence of assay particles. All experiments were done in a 96-well plate, and measurements were taken on the GloMax® plate reader.

FIG. 12A-C. Imaging of a bioluminescent signal on exemplary assay particles. (A) Streptavidin-functionalized porous agarose assay particles were incubated with 0.6 pmole of biotin-HaloTag-NanoLuc and imaged by LV200 imager under steady state. (B) Streptavidin-functionalized porous agarose assay particles were incubated with 0.6 pmole of biotin-HaloTag-NanoLuc imaged by GloMax® Galaxy imager under steady state. (C) Streptavidin-functionalized porous agarose assay particles were incubated with 0.6 pmole of biotin-HaloTag-NanoLuc imaged by Axios Zoom PCO-cooled (EDGE) imager under dynamic flow state.

FIG. 13. Sensitive bioluminescence detection. Streptavidin-functionalized porous agarose assay particles were incubated with an indicated number of biotin-HaloTag-NanoLuc. 20 μM of Furimazine was introduced at steady state conditions, and the assay particles were imaged after 5 minutes. Upper panel represents images at varying absolute amounts of NanoLuc by the LV200 imager, and lower panel represents the same on the Galaxy GloMax® imager.

FIG. 14A-C. Linear quantification of luminescence signal in assay particles. (A) Luminescence signal from 1.7 μL of Biotin-HaloTag-NanoLuc in free solution as per concentrations indicated imaged in parallel in an 0.1 Ibidi μ-slide using 4×4 binning with E7 Retiga camera. (B) Linear standard calibration curve: Y-axis is grey values of bioluminescence and X-axis is number of Biotin-HaloTag-NanoLuc molecules per μL. (C) Streptavidin-functionalized porous agarose assay particles incubated with a concentration of biotin-HaloTag-NanoLuc higher than that used to establish standard curve in (B). The luminescence image shows the signal originating from the beads. To ensure that all the grey values calculated come from the assay particles, background is subtracted. The grey value obtained is plotted on the standard curve, and the concentration is accurately estimated.

FIG. 15A-C. Compatibility with fluorescence and luminescence for imaging-based multiplexing. (A) Streptavidin-functionalized assay particles with fluorescent particles are excited with light to activate each of the four fluorescent dyes contained therein. (B) A combination of four fluorescent dyes were used to create four distinct spectral signatures for subsets of assay particles within the pool of streptavidin-functionalized assay particles. Each spectral signature provides a code that can be used to identify the assay particles within that subset. (C) For the image on the left, all of the assay particles were incubated with biotin-HaloTag-NanoLuc at a concentration of 2.4×10¹¹ molecules per μL and imaged using 4×4 binning with E7 Retiga camera. The image on the right represents an overlay of the luminescence signal with all four unique bead codes pseudo-colored.

FIG. 16A-D. Compatibility of complementation-based bioluminescence with exemplary assay particles. (A) Streptavidin-functionalized porous agarose assay particles were loaded with a synthetic-peptide of Biotin-HiBiT (Promega), followed by washes, incubation with LgBiT (Promega), and subsequent washes. After complementation of the NANOBIT system, 20 μM of Furimazine was added under steady state conditions. (B) Relative luminescence signal over three time points from 4000 Streptavidin-functionalized porous agarose assay particles incubated with increasing amounts of the biotin-HiBiT, and subsequently complemented at a saturating concentration of LgBiT. (C) Similar experiment to (B) but measurements were taken in the absence of biotin-HiBiT (0 pmole biotin-HiBiT; Furimazine only signal) and the absence of LgBiT. (D) Similar experiment to (B), but also carried out with raw porous agarose assay particles without streptavidin functionalization. All experiments were done in a 96-well plate, and measurements were taken on the GloMax® plate reader.

FIG. 17. Complementation-based bioluminescent signal from assay particles can be imaged. Streptavidin-functionalized porous agarose assay particles were incubated with 25 pmole of biotin-HIBIT and 2.5 μM of LGBIT and were imaged by GloMax® Galaxy imager under steady state conditions in the presence of 20 μM Furimazine.

FIG. 18A-D. Complementation-based BRET with exemplary porous agarose assay particles. (A) Streptavidin-functionalized assay particles were loaded with synthetic-Biotin-HiBiT peptide, washed, and subsequently incubated with an engineered circularly permuted (cp) LgBiT chimera that was pre-labeled with chloroalkane-TMR. Following complementation and subsequent washes, 20 μM of Furimazine was added under steady state conditions. The biotin-streptavidin interaction conjugated the NanoBiT onto the assay particle. (B) BRET ratio originated from 4000 assay particles conjugated to biotin-HiBiT and incubated with increasing amounts cpLgBiT chimera. (C) BRET ratio from 4000 assay particles incubated with 25 pmoles biotin-HIBIT and 8 pmoles of cpLgBiT Chimera. To obtain a specific BRET signal for each condition, the cpLgBiT chimera was pre-labeled with increasing amounts of chloroalkane-TMR either in the presence (Blocking) or absence (BRET) of excess chloroalkane biotin. (D) BRET ratio from 4000 assay particles incubated with 25 pmoles of biotin-HiBiT and 4 pmoles of cpLgBiT Chimera. cpLgBiT Chimera for each condition was pre-labeled with increasing amounts of chloroalkane-TMR. All experiments were done in 96-well plates, and measurements were taken on the GloMax® plate reader.

FIG. 19. Imaging of complementation-based BRET signal from assay particles. Streptavidin-functionalized porous agarose assay particles were incubated with 25 pmole of biotin-HiBiT and 8 pmole of cpLgBiT chimera were imaged on GloMax® Galaxy imager under steady state conditions in the presence of 20 μM Furimazine.

FIG. 20. A library of magnetic assay particles with different bead codes (fluorescence labels) and correspondingly different amounts of immobilized luciferase is suspended in oil and exposed to an oil emulsion containing Furimazine. Different grey values (upper image) can be assigned to different amounts of luciferase molecules, which are represented by the bead code (lower image). Streptavidin-functionalized agarose assay particles with unique spectral identities are incubated with varying levels of biotinylated-luciferase, as depicted. The differentially loaded subsets of assay particles are combined and emulsified in a silicone oil to isolate them. The assay particles are then introduced to an emulsion of the luciferase-specific substrate in the same silicone oil formulation used for isolating the assay particles. The substrate is thereby delivered to the isolated assay particles to generate a bioluminescent signal with concordant signal intensity as the amount of luciferase immobilized. The identity of the subsets is confirmed with the unique spectral code.

FIG. 21. Experimental design and results demonstrating compartmentalization of in assay particles using subsets of a population of porous agarose assay particles with different spectral signatures. Assay particles with different codes have been incubated with samples containing different amounts of luciferase. After emulsifying the beads in oil and exposing the bead suspension to an emulsion of Furimazine, the luminescence signals are detected (upper image, grey value representation of signal intensity and thus amount of luciferase in sample) and assigned to the code of each bead, respectively (lower image). The code is then used to identify the sample applied to the respective particles.

FIG. 22. An image of a vial of pellets, each comprising a defined number of dehydrated assay particles (left) and images of rehydrated assay particles (center and right).

FIG. 23. (A) Depiction of an immunoassay embodiment utilizing the technology herein: a first primary antibody is tethered (e.g., by a biotin-streptavidin affinity interaction) to the matrix material; an analyte is added to the assay particles and is bound by the first primary antibody; an untethered primary antibody and secondary antibodies labeled with a luminescent reporter and secondary reporter capable of binding to the first and untethered primary antibodies are added; and wherein upon binding of the primary antibodies to the analyte and binding of the secondary antibodies to the primary antibodies, the luminescent reporter and secondary reporter complement (e.g., via BRET, FRET, structural complementation, etc.) to emit a detectable signal. (B) Deployment of the immunoassay embodiment as described in (A) to detect presence of AKT-1 as an analyte. Immunoassay shows effective complementation of luminescent reporters in the presence of AKT-1 (black bar) over background signal, which is measured using the same detection agents, but merely in the absence of AKT-1 (grey bar). Control experiments were performed in the exact same conditions but without the first primary antibody tethered to the matrix material via biotin-streptavidin affinity interaction. Experiment was done in a 96-well plate, and measurements were taken on the GloMax® plate reader. (C) Complementation-based AKT-1 immunoassay on assay particles as in (B) can be imaged. Streptavidin-functionalized porous agarose assay particles with detection reagent were exposed to AKT-1 as target analyte and imaged by GloMax® Galaxy imager under steady state conditions in the presence of 20 μM Furimazine.

FIG. 24. Graph of luminescence of emulsified NanoLuc®-laden assay particles in the presence of Furimazine in oil (with or without emulsifier).

FIG. 25. Absorbance spectra of silicone oil, Furimazine (Fz) in silicone oil without emulsifier, and Furimazine in silicone oil with emulsifier (10%).

FIG. 26. Microscopy image of oil/emulsifier-dispersed assay particles containing different concentrations of NanoLuc after mixing with Furimazine substrate in an oil-emulsifier emulsion.

FIG. 27. Images of oil/emulsion-dispersed assay particles containing immobilized streptavidin incubated with biotin/Cy5-labeled oligonucleotides in different emulsion carrier compositions.

FIG. 28A-B. Homogenous Fully Particle-Tethered Protein Detection System. (A) Two distinct IL-2 antibodies labeled with biotin and either the SmBiT/LgBiT portion of the luminescent reporter system respectively (B). (A) First IL-2 antibody labeled with SmBiT/biotin and second distinct IL-2 antibody labeled with LgBiT/DBCO to bind orthogonally to the reaction particles via streptavidin and azide, respectively.

FIG. 29A-D: Homogenous two-antibody protein detection system with a single antibody tethered to an assay particle. One antibody specific to the analyte of interest is orthogonally labeled with biotin, and the other antibody labeled with either the (A) SmBiT or (B) LgBiT half of the luminescent reporter system. The antibody is tethered to the reaction particle via Biotin-Streptavidin. Both assay systems showed signal to background ratios similar or above that to the commercial luminescent Lumit® IL-2 detection system (C) when comparing RLU on a plate reader. (D) The system depicted in (B) is exemplified with 10% pooled human Serum or Plasma.

FIG. 30A-C. Standard curve and picogram levels of lower limit of detection with an image-based homogenous protein detection reaction particle system. (A) Reaction particle protein detection system. (B) IL-2 analyte was spiked into cell media to generate a standard curve at indicated quantities, and the reaction particle protein detection system components were added to the cell media, after 1.5 hrs of incubation, substrate was added, reaction particles were imaged, and luminescent signal was quantified. (C) IL-2 standard curve using quadratic fit equation and lower limit of detection calculated using 3 standard deviation of background showing pg/mL levels of sensitivity as well as R2 value of >0.99.

FIG. 31A-G. Quantitative multiplexing of standard curves on a homogenous reaction particle protein detection system. (A) A schematic depicting the concept of multiplexing a protein standard curve in order to decrease the number of wells needed to be used/imaged. The protein detection reaction particle system used for (B) IFNΥ and (C) IL-2. (D) Fluorescent and luminescent images were taken to obtain reaction particle code and correlating luminescent reporter signal. (E) Two different populations of reaction particle codes as well as two different signal intensities of reporter luminescent signal are observed that correlate with each analyte IFNΥ (right) or IL-2 (left). (F/G) Using the multiplexed standard curves generated, the multiplexed sample concentration was calculated to be within <15% of the expected concentration exemplifying the ability to quantitatively multiplex a homogenous reaction particle protein detection system.

FIG. 32A-D. Expansion of Multiplexing Using a Homogenous Reaction Particle Protein Detection System using 5 different analytes. (A) A reaction particle protein detection system used for 5 different analytes. (B) Depicted are the 5 reaction particles codes used for each analyte (B top panel): IL-2/Cy3, IL-6/Cy3- and Cy5, IL-10 Cy5 and Cy7, IFNΥ/No label, TNFα/No label, lower optical density. Shown is the example of luminescent signal seen for each analyte in the presence (B, middle panel) and absence (B, bottom panel) of analyte. (C) All 5 different reaction particles, 5 different exogenous antibodies, and 5 different analytes were mixed and added together simultaneously, incubated, and imaged for reaction particle code and corresponding luminescence. (D) In order to show specificity of the multiplex, all 5 detection reagents were mixed together, and only 4 of the 5 analytes were added.

FIG. 33A-C. Using Raman detection for Multiplexing Reaction Particles. (A) Three different Raman reporters were embedded separately in polystyrene beads and identified in the mixture of the three. (B) The three different Raman reporters embedded polystyrene beads were imaged in the presence of NanoBRET reaction containing 6 nM NanoLuc-HaloTag, 300 nM Janelia Fluor® 585 HaloTag® ligand, and 20 μM Furimazine showing the ability for the Raman reporters to be imaged in the presence of luminescent, BRET, and fluorescent signal. (C) Signals from three different reaction particles including a negative control which showed no signal at the wavenumber of interest.

FIG. 34. Bioluminescent reporters are compatible with porous assay particles made of different polymer matrices.

FIG. 35A-B. Complementation-based bioluminescence assays are compatible with porous assay particles made of different polymer matrices. (A) Cartoon depiction of NanoBiT® complementation assay using a mCherry-GFP dimer as analyte with GFP, and mCherry nanobodies (VHH) fused to NanoBiT components as detection agents. The GFP VHH is the first detection agent and is conjugated to the bead using biotin-labelled HaloTag (HT). The mCherry VHH with unlabelled HaloTag (HT) is the second detection agent and is subsequently added homogenously with analyte. Upon substrate addition, the bioluminescent signal is confined to the assay particle due to analyte dependent complementation of NanoBiT® components and chemical tethering of the first detection agent leveraging HT. (B) Examples of the bioluminescent complementation assay described in (A) using assay particles that are made of natural polymers (agarose and alginate), synthetic polymers (acrylamide; PNIPAAm), and co-polymers (gelatine-agarose).

FIG. 36A-B. Compatibility of intramolecular, bioluminescent protease-based cleavage assay with exemplary assay particles. (A) Depiction of circularly permuted NanoLuc® and NanoBiT® biosensors in which the binary component bioluminescent reporter is separated by a linker which contains a specific protease recognition site. (B) Examples of separate caspase-3 and TEV proteases biosensors as described in (A).

FIG. 37. Intramolecular, multiplexed bioluminescent protease-based cleavage assay with exemplary assay particles.

FIG. 38. Non-NanoLuc-based reporters are compatible with exemplary assay particles.

FIG. 39A-B. Bioluminescent metabolite assays. (A) Streptavidin-functionalized assay particles were loaded with Ultra-Glo biotinylated via cysteines (leveraging maleimide chemistry) and Glutamate Dehydrogenase biotinylated via lysines (leveraging succinimide chemistry). In the presence or absence glutamate (analyte), luciferin detection agent and Reductase enzyme is concurrently added homogenously. The three-enzyme chained bioluminescent assay specifically detects presence of the glutamate metabolite as a bioluminescent signal confined to the porous assay particles. (B) Modification of (A) where all three enzymes are loaded onto the assay particles. Streptavidin-functionalized assay particles were loaded with Ultra-Glo biotinylated via cysteines (leveraging maleimide chemistry), Glutamate Dehydrogenase biotinylated via lysines (leveraging succinimide chemistry), and Reductase biotinylated via cysteines (leveraging maleimide chemistry. In the presence or absence glutamate (analyte), luciferin detection agent is concurrently added homogenously. The three-enzyme chained bioluminescent assay specifically detects presence of the glutamate metabolite as a bioluminescent signal confined to the porous assay particles.

FIG. 40A-B. (A) An immunoassay embodiment in which a first primary antibody is tethered to the matrix material; an analyte is added to the assay particles and is bound by the first primary antibody; an untethered primary antibody and secondary antibodies labeled with a luminescent reporter and secondary reporter capable of binding to the first and untethered primary antibodies are added; upon binding of the primary antibodies to the analyte and binding of the secondary antibodies to the primary antibodies, the luminescent reporter and secondary reporter complement (e.g., via BRET, FRET, structural complementation, etc.); resultant assay particles are emulsified into a non-aqueous environment using silicone oil with emulsifier (10%); the Furimazine substrate is separately emulsified in an oil-emulsifier mixture; and wherein emulsified Furimazine is delivered to the isolated assay particles in a non-aqueous environment to produced detectable signal in presence of analyte. (B) Deployment of the immunoassay embodiment to detect presence of AKT-1 as an analyte in a non-aqueous environment. Immunoassay shows effective complementation of luminescent reporters in the presence of AKT-1. Streptavidin-functionalized porous assay particles with detection reagent were exposed to AKT-1 as target analyte, emulsified, and imaged under steady state conditions in the presence of emulsified Furimazine.

FIG. 41A-C. Bioluminescent target engagement assays deploying Bioluminescence Resonance Energy Transfer (BRET) are compatible with exemplary assay particles. (A) Comparison of a NanoBRET® target engagement assay for Cyclin G-Associated Kinase (GAK) in live cells and assay particles. In live cells, NanoLuc® (Nluc)-GAK-HaloTag® (HT) fusion protein is overexpressed and labelled with a dose response of GAK-specific K-10 tracer (BRET acceptor). In presence of Furimazine, Nluc bioluminescent (donor) signal is transferred to the acceptor and detected as BRET. Upon treatment with unlabeled kinase inhibitor CTx-0294885 (drug), BRET to the K10-tracer is abrogated, as expected. In porous assay particles conjugated with chloroalkane, protein fusion of Nluc-GAK-HT is loaded from cell lysates onto the particles via the HT handle. Dose response of K-10 tracer as a BRET acceptor and abrogation of BRET signal in presence of unlabeled drug, as seen in live cells, is recapitulated in the porous assay particles. Excess K-10 tracer doesn't lead to an increase in collisional (non-specific) BRET since assay particles loaded with the fusion construct of Nluc-HT without GAK shows insignificant BRET transfer. This indicates that the BRET signal is specific to the kinase. Experiment was done in a 96-well plate, and measurements were taken on the GloMax® plate reader. (B) Imaging of the Cyclin G-Associated Kinase (GAK) target engagement assay in assay particles as described in (A) using the GloMax® Galaxy imager. K-10 Tracer acceptor (1 μM) is used to label the Nluc-GAK-HT fusion protein, and effective BRET is seen in presence of Furimazine. BRET is abrogated when the assay particles are treated with unlabeled kinase inhibitor (drug: 10 μM of CTx-0294885). Assay was performed in an aqueous format for homogenous delivery of tracer and drug. (C) Imaging of the Serine/threonine-protein kinase 16 (STK16) target engagement assay in assay particles as described in (B) using the GloMax® Galaxy imager. K-10 Tracer acceptor (1 μM) is used to label the Nluc-STK16-HT fusion protein, and effective BRET is seen in presence of Furimazine. BRET is abrogated when the assay particles are treated with unlabeled kinase inhibitor (drug: 10 μM of CTx-0294885). Assay was performed in an aqueous format for homogenous delivery of tracer and drug.

FIG. 42A-B. Bioluminescent target engagement assays deploying Bioluminescence Resonance Energy Transfer (BRET) quantified on exemplary assay particles. (A) Quantified imaging of the Cyclin G-Associated Kinase (GAK) and Serine/threonine-protein kinase 16 (STK16) NanoBRET® target engagement assay in assay particles using the GloMax® Galaxy imager. K-10 Tracer acceptor (1 μM) is used to label the NanoLuc (Nluc)-GAK-HaloTag (HT), Nluc-STK16-HT, and Nluc-HT fusion proteins, and effective BRET is seen in presence of Furimazine. BRET is abrogated when the assay particles are treated with unlabeled kinase inhibitor (drug: 10 μM of CTx-0294885). Assay was performed in an aqueous format for homogenous delivery of tracer and drug. (B) Plate-based quantification on a GloMax® plate reader of the assays performed as described in (A) that shows kinase specific inhibition of BRET upon drug treatment.

FIG. 43A-C. Using reaction particle size as a system for coding and multiplexing. (A) reaction particle protein detection system. (B) The homogenous IL-2 protein reaction particle detection system was employed on the particles on all three sizes showing luminescent signal on each type of bead. The different particle sizes, as well as a difference of optical density allowed for a 4-analyte multiplex while only image the codes in brightfield (C, top panel). The corresponding codes were as follows: IL-2/70 μm, IL10/90 μm, TNFα/120 μm, IFNΥ/90 μm/low optical density. All the reaction particles for each analyte, the corresponding exogenous 2nd Bit-labeled antibodies as well as all 4 analytes mixed together.

FIG. 44. Using Fluorescence and Reaction Particle Size to generate >20 codes. Three particle sizes were labeled with combinations of 3 different fluorescent dyes to generate >20 reaction particle codes. All reaction particles were mixed together and imaged in brightfield and fluorescent channels to identify the codes.

FIG. 45. Real-Time Imaging of IL-2 binding to the Homogenous Reaction Particle Protein Detection System. The homogenous protein binding assay was set up as shown in the schematic. The components of the assay, reaction particle with tethered IL-2 SmBiT antibody, in-solution IL-2-LgBiT antibody and luminescent substrate were added to a well containing IL-2, briefly mixed and immediately imaged for luminescence at 15-minute time intervals. An increase in luminescent signal is shown over time showing the ability to image binding of IL-2 to the protein reporter system kinetically in real-time.

FIG. 46A-B. The ability to incubate the reaction particle homogenous protein detection system with primary cells and detect kinetic and real-time endogenous protein production. (A) Reaction particles tethered with IL-2 SmBiT antibody and IL-2-LgBiT antibody un-tethered were added to wells containing Peripheral Blood Mononuclear Cells (PBMCs). PBMCs were then stimulated with either 1× Cell Stimulation Cocktail (CSC) or vehicle control. Shown are brightfield and luminescent images of the reaction particles with PBMCs at various time points post stimulation with CSC. Luminescent substrate was added to a different well at each time point and imaged. Shown is the ability to detect an increase of endogenous IL-2 production over time. (B) Cells and assay were setup as in (A) but 1.5 hrs post assay component addition and CSC stimulation, substrate was added and the same well was continuously imaged over the course of 5 hrs detecting endogenous IL-2 production in real-time.

FIG. 47. Compound profiling of kinase inhibitors against cyclin G-associated kinase (GAK)

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a peptide” is a reference to one or more peptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “particle” refers to a small object (e.g., dimensions of <1 mm, <500 μm, <250 μm, <100 μm, etc.) to which can be ascribed physical properties such as volume, mass average size, etc. Particles may accordingly be of a symmetrical, globular, essentially globular or spherical shape, or be of an irregular, asymmetric shape or form. In certain embodiments, the particle is spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. The size of a particle may vary from dimensions of low nanometers to hundreds of micrometers (e.g., 1 nm, 2 nm, 5 nm, 10 nm, 100 nm, 200 nm, 500 nm, 1 μm, 2 μm, 5 μm, 10 μm, 100 μm, 200 μm, 500 μm, and ranges and values therebetween).

As used herein, the term “microparticle” refers to particles with a greatest dimension (e.g., diameter) in the nanometer and micrometer range (e.g., 100 nm, 200 nm, 100 nm, 200 nm, 500 nm, 1 μm, 2 μm, 5 μm, 10 μm, 100 μm, 200 μm, 500 μm).

As used herein, the term “bead” refers to a particle that is generally spherical or spheroid in shape. A “microbead” is a generally spherical or spheroid microparticle.

As used herein, the term “nanoreactor” refers to a synthetic or biological chemical reactor that operates on the nanometer scale to create unique chemical environments that are different from the surrounding bulk space.

As used herein, the term “prefabricated” refers to a composition or entity (e.g., a particle) that is generated prior to and/or separate from its use in an assay or reaction mixture. A prefabricated composition or entity (e.g., particle) is not generated during use or in the mixture/solution within which it will be used. For example, a prefabricated particle according to certain embodiments herein is not an in-situ generated particle, i.e., it is not a particle that is generated in the course of a reaction in which it is intended to be used (e.g., an analytic assay). Conversely, the term “in-situ generated,” as used herein, refers to a composition or entity (e.g., a particle) that is generated from one or more precursor components within a volume that is intended for the use of the composition or entity (e.g., a particle), such as an assay reaction mixture. A prefabricated microparticle, in accordance with embodiments herein, does not, at the time of it being generated, contain, incorporate, or engulf a sample containing an analyte to be detected. Rather, a prefabricated composition or entity (e.g., particle) in accordance with embodiments herein is generated in one or more steps conducted outside of and/or away from a sample/analyte to be analyzed. Optionally, post-fabrication, a prefabricated composition or entity (e.g., particle) is exposed to a sample containing an analyte or suspected of containing an analyte. In some embodiments, one or more post-fabrication steps (e.g., liquefication, rehydration, dispersal in a non-aqueous medium, etc.) may be conducted (e.g., within an analyte-containing sample) with a prefabricated composition or entity (e.g., particle), that alter its structure, composition, component elements, etc.

As used herein, the term “homogeneous,” when used in reference to a physical structure (e.g., a homogeneous assay particle), means having a substantially uniform composition throughout the structure, with materials, pores, and other components substantially uniformly dispersed throughout the structure. A structure (e.g., assay particle) may exhibit a homogeneous global structure in which the constituent materials are uniformly distributed throughout the volume of the structure, while having irregular or random local morphology, for example in the shape of pores and polymer network. A homogenous structure does not contain morphologically distinct domains, such as a core and shell of distinct material composition.

When used in reference to an assay or other method (e.g., a homogeneous assay), the term “homogeneous” refers to a single-step workflow without separation or wash steps that can be conducted in a single vessel. A heterogeneous assay requires multiple steps, separation of components (e.g., to remove unbound or interfering components), and possibly wash steps.

As used herein, the term “interstitial” refers to an intervening space existing between structures (e.g., within matrix material). An “interstitial void volume” does not refer to an open cavity but instead a network of open space interwoven through a material.

As used herein, the term “fluid communication” refers to the ability of fluid (e.g., aqueous solution) and any solutes, small molecules, and macromolecules therein to freely move between two or more components, volumes, etc. The term does not imply directionality. As used herein, the term “equivalent,” when used is connection with the particles herein, refers to scenarios in which each particle of a population, whether being of the same or different subsets, provides substantially the same internal volume and conditions (other than defined different components of particles in different subsets) and is capable of encompassing substantially the same number of target analytes when placed in the same sample. The equivalent particles are a plurality of like volumes (e.g., reaction spaces) for each target analyte type, in which the individual concentrations of target analytes and other sample components are substantially the same amongst different particles. In some embodiments, assay particles of different subsets of a population are equivalent particles (e.g., substantially the same volume, internal conditions, materials, etc.), despite having, for example, different target-specific interaction moieties, spectral signatures, etc.

As used herein, the term “porous” refers to the characteristic of a material or object having pores or minute spaces, ducts, recesses, tunnels, bores, or holes through which liquids and other molecules may pass. A porous material or structure (e.g., an assay particle) is a continuous matrix of materials containing a network of interconnected pores distributed throughout its volume. Although the pores and resulting void volume may comprise a significant portion of the volume of a porous structure, the pores are distributed within a matrix. The “porosity” of a material or object refers to the allowance of the uptake of liquid and of analytes present in said liquid into a porous particle (e.g., through or into the space provided for by the pores) and may be described in relation to the type or size of molecules that may pass through the pores (e.g., porous to small molecules, porous to macromolecules, etc.). The porosity may also be defined as the average amount of non-solid space contained in a material (e.g., an assay particle). Such space is considered interstitial void of volume even if it contains liquid (e.g., water, buffer, a sample, etc.). Porosity or void volume of a particle can be defined as the ratio of the total volume of the pores (i.e., void volume) in the material to the overall volume of particle. In some embodiments, porosity, defined as the volume fraction pores, can be calculated from particle density, which can be measured, for example, gravimetrically. In some embodiments, assay particles herein have a porosity of at least about 20%, at least about 30%, at least about 40%, at least about 50%, at least about 60%, at least about 70%, at least about 80%, at least about 90%, or more than 90%.

The terms “core-shell” and “hollow core” as used herein refer to a structure having a contiguous central cavity enclosed by a surrounding exterior wall, shell, envelope, etc. without a significant amount of the exterior material distributed within the cavity.

As used herein, the term “porogen” refers to a material that is used to make pores in another material or object (e.g., an assay particle). Following formation of the other material or object, the porogen is removed (e.g., by diffusing, dissolving, melting, degrading, etc.) to obtain a porous structure in which pores and/or an interstitial void volume is formed at the portions previously occupied by the porogen.

As used herein, the term “matrix” refers to a three-dimensional network of material that provides and supports the body of a particle. Pores within the network or material provide a void volume within the particle. The structural parameters of the pores within the matrix, including the pore size, pore interconnectivity/tortuosity and surface area, can affect how substances (e.g., fluid, solutes, analytes, macromolecules, etc.) access and move about the particle.

As used herein, the term “void volume” refers to the internal space within a particle or other object that is available to be occupied by a sample.

As used herein, the term “sample” is used in its broadest sense and refers to any material, substance, mixture, or portion thereof from which one or more analytes, signals, properties, and/or the absence thereof may be measured, detected, processed, or analyzed. The term sample also includes a specimen or culture obtained from any source, an assay or reaction mixture, as well as biological and environmental samples. The term “sample” is meant to also include any material, substance, composition, mixture, or portion thereof, which is suspected of containing an analyte, and/or which is suspected of being amenable to being subjected to a measurement, detection, analysis, or processing step. A sample may contain an analyte to be detected, may be suspected of containing an analyte to be detected, or may be assayed to determine whether it contains an analyte. An analyte may be present or absent in a sample. Biological samples may be obtained from animals (including humans) and encompass fluids, solids, tissues, and gases, and may include urine, saliva, semen, and blood products such as whole blood, plasma, serum, and the like. A sample may also refer to a live sample, such as cells, tissue, whole organisms, and/or material excreted from a cell, cell lysates, purified forms of the enzymes, peptides, and/or polypeptides described herein. A sample may also include cell-free expression systems. Environmental samples include environmental material such as surface matter, soil, water, crystals, wastewater, and industrial samples. Such examples are not, however, to be construed as limiting the sample types applicable to the embodiments herein.

As used herein, the term “target analyte” refers to a substance being identified, measured, quantified, isolated, etc., in a system, method, sample, or assay described herein. An analyte may be a small molecule, peptide, protein, nucleic acid, lipid, complex, or other suitable molecular, macromolecular, or supramolecular entities.

As used herein, the term “biocompatible” refers to the capacity of an entity or material to be tolerated by cells (e.g., prokaryotic or eukaryotic) and/or more complex biological systems (e.g., tissues, organisms) and the components thereof (e.g., proteins, antibodies, nucleic acids, etc.) by being nontoxic toward such systems and components.

As used herein, the term “polymer” refers to a material or a class of natural or synthetic substances composed of large molecules (e.g., macromolecules), which are multiples of simpler chemical units (e.g., monomers). Within the scope herein a polymer may be a homopolymer (multiples of a single type of monomer unit) or copolymer (formed by linking two or more different types of monomer units) and may be linear or branched.

As used herein, the term “natural polymer” refers to polymer substances and materials that occur in nature, such as those including but not limited to, polysaccharides (e.g., agarose, chitin, chitosan, alginate, carrageenan, cellulose, fucoidan, laminaran, gums selected from xanthan gum, arabic gum, ghatti gum, guar gum, locust bean gum, tragacanth gum, karaya gum and inulin), polypeptides (e.g., albumins, collagens, gelatins), and polynucleotides.

As used herein, the term “synthetic polymer” refers to polymers that have been artificially prepared from their corresponding monomers, such a monomers including but not limited to methylacrylate and acrylate, acrylamide and methacrylamide, cyclic lactams, styrene-based monomers, etc.

As used herein, the term “hydrogel” refers to a three-dimensional polymeric structure that is insoluble or minimally soluble in water or other aqueous solutions, but which is capable of absorbing and retaining water or other aqueous solutions from the adjacent environment while remaining a stable structure. A hydrogel is typically formed when an organic polymer (natural or synthetic) is cross-linked via covalent, ionic, or hydrogen bonds to create a three-dimensional open-lattice structure that entraps water molecules to form a gel.

As used herein, the term “emulsion” refers to a fine dispersion of minute droplets of one liquid in another liquid in which it is not soluble or miscible (e.g., a mixture of two or more liquids that are normally immiscible (i.e., unmixable or unblendable) due to liquid-liquid phase separation).

As used herein, the terms “tethering” or “tethered” refer to stably connecting or linking two entities by any suitable covalent or non-covalent linkage. A first protein, polypeptide, or peptide entity can be tethered to a second protein, polypeptide, or peptide entity by genetic fusion (e.g., directly or via a peptide linker). The entities can be tethered by the non-covalent interaction of affinity agents/entities attached. The entities can be tethered by a covalent linkage formed between reactive agents/moieties on each entity (e.g., click chemistry, HALOTAG, etc.). Unless specified otherwise, embodiments herein are not limited by the type of tether or the types of entities (e.g., small molecule, peptide, protein, antibody, lipid, etc.) that are tethered together.

As used herein, the term “irreversibly tethered” refers to two entities that are joined by a bond or linkage that does not dissociate under the conditions of use, and does not contain a feature intended for the separation of the two entities (e.g., cleavage site) such that the entities remain physically associated as a single complex throughout the intended operation of the system. The linkage may be covalent or otherwise non-dissociable except under harsh denaturing or cleavage conditions that are not encountered in the claimed assay, reaction, or environment.

As used herein, the term “linker” refers to a chemical moiety that serves as a covalent tether between the two entities. A linker may be a peptide (e.g., in the case of a fusion of two peptide/polypeptide entities), a carbon chain (e.g., optionally interrupted and/or substituted by one or more nitrogen atoms, oxygen atoms, carbonyl groups, rings, or other functional groups), a polymer (e.g., PEG), or any other chemical moiety capable of tethering two entities. A linker may tether a chemical moiety (e.g., reactive group, fluorophore, affinity label, etc.) to a component of an assay system (e.g., antibody, protein target, analyte, etc.). Unless specified otherwise, embodiments herein are not limited by the type of tether or the types of entities (e.g., small molecule, peptide, protein, antibody, lipid, etc.) that are tethered together.

As used herein, the term “cleavable linker” refers to a chemical moiety (e.g., a linker) that can be severed in a controlled manner to release the linked molecular entities from each other. For example, a cleavable linker can be enzymatically cleavable (e.g., by a protease), a chemically cleavable, or photocleavable.

As used herein, the term “affinity” refers to the non-covalent attraction between two moieties (e.g., an affinity agent (e.g., streptavidin) and an affinity label (e.g., biotin)). Binding of the two affinity groups (e.g., agent and label) can be used to tether other molecular entities attached thereto.

As used herein, the term “binding agent” refers to a refers to a molecular entity capable of binding, typically non-covalently, to an analyte (e.g., specifically or non-specifically). Exemplary binding agents include, but are not limited to antibodies, antibody fragments, natural target binding domains, a computationally derived binding domain, affinity molecules, affimers, aptamers, DARPins, ligands, and proteins (e.g., proteins capable of binding to a ligand).

As used herein, the term “antibody” refers to a whole antibody molecule or a fragment thereof (e.g., fragments such as Fab, Fab′, and F(ab′)₂, variable light chain, variable heavy chain, Fv). It may be a polyclonal or monoclonal or recombinant antibody, a chimeric antibody, a humanized antibody, a human antibody, etc. As used herein, when an antibody or other entity “specifically recognizes” or “specifically binds” an antigen or epitope, it preferentially recognizes the antigen in a complex mixture of proteins and/or macromolecules and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “antibody fragment” refers to a portion of a full-length antibody, including at least a portion of the antigen binding region or a variable region. Antibody fragments include, but are not limited to, Fab, Fab′, F(ab′)₂, Fv, scFv, Fd, variable light chain, variable heavy chain, diabodies, and other antibody fragments that retain at least a portion of the variable region of an intact antibody. See, e.g., Hudson et al. (2003) Nat. Med. 9:129-134; incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis. For example, a “Fab” fragment comprises one light chain and the CHI and variable region of one heavy chain. The heavy chain of a Fab molecule cannot form a disulfide bond with another heavy chain molecule. A “Fab” fragment comprises one light chain and one heavy chain that comprises an additional constant region extending between the C_{H1} and C_{H2} domains. An interchain disulfide bond can be formed between two heavy chains of a Fab′ fragment to form a “F(ab′)₂” molecule. An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; incorporated by reference in their entireties. In certain instances, a single variable region (e.g., a heavy chain variable region or a light chain variable region) may have the ability to recognize and bind antigen. Other antibody fragments will be understood by skilled artisans.

As used herein, the term “luminescence” refers to the emission of light from an agent or substance without incandescence (i.e., not as the result of heat). Examples of luminescence include fluorescence, bioluminescence, and chemiluminescence.

As used herein, the term “fluorescence” refers to the emission of electromagnetic radiation (e.g., light) by an agent or substance at a emission wavelength as a result of excitation of the agent or substance by irradiation at a different excitation wavelength.

As used herein, the term “bioluminescence” refers to production and emission of light by a chemical reaction catalyzed by, or enabled by, an enzyme, protein, protein complex, or other biomolecule (e.g., bioluminescent complex). In typical embodiments, a substrate for a bioluminescent entity (e.g., bioluminescent protein or bioluminescent complex) is converted into an unstable form by the bioluminescent entity; the substrate subsequently emits light as it returns to its stable form.

As used herein, the term “chemiluminescence” refers to production and emission of light by a non-enzymatically (or otherwise biologically-catalyzed) chemical reaction.

As used herein, the term “non-fluorescence-based luminescence” refers to an emission of radiation that is not the result of an excitation of an agent by irradiation (e.g., from the outside of the one or more assay particles). In preferred embodiments, non-fluorescence-based luminescence refers to an emission of radiation as a result of a reaction, in particular of a chemical or biological or biochemical reaction (e.g., that takes place within the one or more assay particle(s)). In even more preferred embodiments, “non-fluorescence-based luminescence” is selected from “bioluminescence” and “chemiluminescence.”

As used herein, the term “luminophore” refers to a chemical moiety or compound that can be placed in an excited electronic state (e.g., by a chemical or enzymatic reaction) and emits light as it returns to its electronic ground state.

As used herein, the term “source-resolved detection” refers to a process of detecting or quantitating optical, molecular, physical, or other detectable features of signals while preserving information regarding the source of the feature/signal. Source-resolved detection allows for two different features or signals emitted (e.g., luminescent reporter signal, assay particle-identifying physical characteristic, etc.) from the same source (e.g., same assay particle) to be attributed to that source. Techniques providing source-resolved detection allow for detected features/signals to be assigned to a source of origin and for separate signals (e.g., reporter signal and assay-particle-identifying physical characteristic) originating from the same source to be correlated. Exemplary techniques capable of source-resolved detection may include imaging of microscopy techniques, flow cytometry, mass cytometry, x-ray fluorescence/spectroscopy, atomic absorption, Coulter counting, atomic force microscopy, optical tweezers, magnetic particle spectroscopy, microfluidic detection, and hybrid (multimodal) techniques.

As used herein, the term “spatially resolved signal acquisition” refers to a process of detecting or quantitating optical, molecular, physical, or other detectable signals while preserving information regarding the positional context and/or spatial origin of the signals within a sample or detection landscape. Techniques providing spatially resolved signal acquisition allow for detected signals to be assigned to a location of origin and for separate signals (e.g., reporter signal and assay-particle-identifying physical characteristic) originating from the same location to be correlated.

As used herein, the term “multiparametric” refers to the detection and/or measurement of multiple distinct parameters for the same individual entity in a single experiment or observation. For example, the size of a particle, a fluorescent signal from the particle, and a bioluminescent signal from the particle may all be detected. In some embodiments, multiparametric detection is coupled with source-resolved detection and/or spatially resolved signal acquisition to provide assignment of multiple signals/features to the same source and/or location.

As used herein, the term “multiplex” refers to conducting two or more analogous sub-assays (e.g., detecting/quantitating two or more analytes using the same assay architecture) simultaneously in a single system or sample. As used herein, the term “single-reporter multiplex” refers to an assay capable of multiplex detection/quantification using the same reporter (or reporter system (e.g., RET, complementation, etc.)) while differentiating the sub-assays by an independent signal or feature.

As used herein, the term “assay-particle-identifying physical characteristic” refers to any optical, molecular, physical, or other detectable/measurable features of an assay particle that can be used to differentiate the assay particle from other assay particles bearing different features. Assay-particle-identifying physical characteristics may be inherent to the particle (e.g., size, shape, dimensions, composition, optical density, signal emissions, etc.), may be by virtue of a tag, label, or assay-particle-identifying signal generator (e.g., fluorescent, a colorimetric, a Raman, an infrared, a chemiluminescent, bioluminescent, etc. tag, label, etc.) and/or a combination of any suitable features. In some embodiments, an assay-particle-identifying physical characteristic is or comprises a spectra signature.

As used herein, the term “spectral signature” refers to an encoded component or set of components within an entity (e.g., a particle) that is suitable for emitting a detectable signal that is unique to the particular entity and/or unique to entities having the same molecular makeup or components. The spectral signature can be correlated to the molecular makeup or components of the entity, used to identify the entity, and/or used to differentiate or group entities within a collection of different entities. Detectable signature signals emitted from a spectral signature may include but are not limited to a fluorescent signal, a colorimetric signal, a Raman signal, an infrared signal, a chemiluminescent signal, and a bioluminescent signal. For example, a spectral signature may be a particular combination of a fluorophores, such that detection of the fluorescent signal from that fluorophore combination can be used to identify the particle (and the components thereof) known to contain that spectral signature.

As used herein, the term “imidazopyrazine luminophore” refers to a genus of luminophores including “native coelenterazine” as well as synthetic (e.g., derivative or variant) and natural analogs thereof, including Furimazine, Furimazine analogs (e.g., fluoroFurimazine, hydroFurimazine) coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); U.S. Pat. No. 8,669,103; U.S. Prov. App. No. 63/379,573; the disclosures of which are incorporated by reference in their entireties.

As used herein, the term “coelenterazine” refers to the naturally-occurring (“native”) imidazopyrazine of the structure:

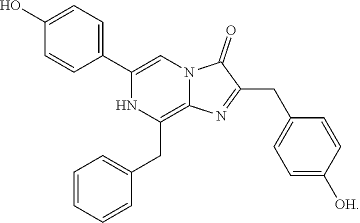 [SMILES: O=c1c(Cc2ccc(O)cc2)nc2c(Cc3ccccc3)[nH]c(-c3ccc(O)cc3)cn1-2]

As used herein, the term “Furimazine” refers to the coelenterazine derivative of the structure:

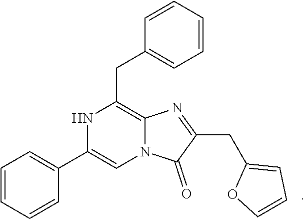 [SMILES: O=c1c(Cc2ccco2)nc2c(Cc3ccccc3)[nH]c(-c3ccccc3)cn1-2]

As used herein, the term “fluoroFurimazine” refers to the Furimazine derivative of the structure:

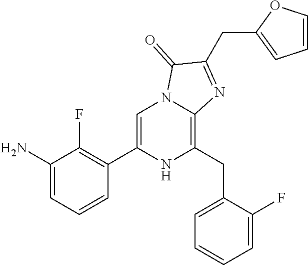 [SMILES: Nc1cccc(-c2cn3c(=O)c(Cc4ccco4)nc-3c(Cc3ccccc3F)[nH]2)c1F]

(U.S. application Ser. No. 16/548,214; incorporated by reference in its entirety).

As used herein, the term “resonance energy transfer” (“RET”) refers to the non-radiative transfer of energy from an excited donor molecule to an acceptor molecule through dipole-dipole interactions. RET occurs when the emission spectrum of the donor overlaps with the absorption spectrum of the acceptor. The transfer efficiency depends on the distance between the donor and acceptor and the orientation of the dipoles.

As used herein, the term “bioluminescence resonance energy transfer” (“BRET”) refers to the distance-dependent interaction in which energy is transferred from a donor bioluminescent protein/complex and substrate to an acceptor molecule without emission of a photon. The efficiency of BRET is dependent on the inverse sixth power of the intermolecular separation, making it useful over distances comparable with the dimensions of biological macromolecules (e.g., within 30-80 Å, depending on the degree of spectral overlap).

As used herein, the term “Forster resonance energy transfer” (“FRET”) refers to the non-radiative transfer of energy from an excited donor fluorophore to an acceptor fluorophore, which then in turn can emit the received energy in the form of fluorescence.

As used herein, the term “Oplophorus luciferase” (“OgLuc”) refers to a luminescent polypeptide having significant sequence identity, structural conservation, and/or the functional activity of the luciferase produced by and derived from the deep-sea shrimp Oplophorus gracilirostris. In particular, an OgLuc polypeptide refers to a luminescent polypeptide having significant sequence identity, structural conservation, and/or the functional activity of the mature 19 kDa subunit of the Oplophorus luciferase protein complex (e.g., without a signal sequence) such as SEQ ID NOs: 1 (NANOLUC), which comprises 10 β strands (β1, β2, β3, β4, β5, β6, β7, β8, 39, β10) and utilize substrates such as coelenterazine or a coelenterazine derivative or analog to produce luminescence.

As used herein the term “complementary” refers to the characteristic of two or more structural elements (e.g., peptide, polypeptide, nucleic acid, small molecule, etc.) being able to hybridize, dimerize, or otherwise form a complex with each other. For example, a “complementary peptide and polypeptide” are capable of coming together to form a complex. Complementary elements may require assistance (facilitation) to form a complex (e.g., from interaction elements), for example, to place the elements in the proper conformation for complementarity, to co-localize complementary elements, to lower interaction energy for complementary, to overcome low affinity for one another, etc.

As used herein, the term “complex” refers to an assemblage or aggregate of molecules (e.g., peptides, polypeptides, etc.) in direct and/or indirect contact with one another. In one aspect, “contact,” or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules (e.g., peptides and polypeptide) is formed under assay conditions such that the complex is thermodynamically favored (e.g., compared to a non-aggregated, or non-complexed, state of its component molecules). As used herein, the term “complex,” unless described as otherwise, refers to the assemblage of two or more molecules (e.g., peptides, polypeptides, or a combination thereof).

As used herein, the term “target engagement assay” refers to a method of measuring the binding of target molecule to a molecule of interest (e.g., drug molecule). Bioluminescence resonance energy transfer (BRET), fluorescence anisotropy, and thermal shift assays are examples of techniques that can be used to monitor (e.g., quantify) binding.

As used herein, the term “target mimetic” refers to a molecular entity that is capable of simulating the binding of a target analyte to a binding partner. The target mimetic is capable of binding to the binding partner with substantially similar affinity and to the same binding pocket and/or interface as the target analyte.

As used herein, the term “system” refers to a collection of compositions grouped together in any suitable manner (e.g., physically associated, within the same fluid (e.g., reaction mixture, cell lysate, etc.), body (e.g., cell), packaged together (e.g., in a kit), etc.) for a particular purpose.

As used herein, the term “substantially” means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., substantially non-luminescent) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic (e.g., luminescent intensity of a bioluminescent protein or bioluminescent complex).

### DETAILED DESCRIPTION

Provided herein are assay particles comprising a matrix material, interstitial void volume, and reagents for target-specific luminescent detection.

Provided herein are luminescent reporter platforms that, in some embodiments, allow for multiplexed profiling of target analytes with flexible formatting and minimal impact on complex biological models being interrogated. Embodiments herein offer significant advantages over traditional reporters in molecular biology and biochemical assays.

Described herein is a platform technology that comprises assay particles that may be composed of various materials. In some embodiments, homogeneous assay particles are provided in which an interstitial void volume exists within a matrix material that is substantially evenly dispersed throughout the assay particle. Various assay components can be attached to the matrix material that comprises the assay particles and/or contained within the interstitial void volume, and different assays (e.g., detection of target analytes (e.g., non-natural analytes, synthetic analytes, bioengineered analytes, molecular assemblies, antibodies, nucleic acids, metabolites, cells, drugs, pathogens, molecules, metals, etc.)) can be conducted on or within the assay particles depending on the specific components. Different luminescent reporters (e.g., luciferases, bioluminescent complexes and components thereof, fluorophores, FRET systems, BRET systems, etc.) find use with the assay particles herein. Different target-specific interaction moieties (e.g., antibodies, antibody fragments, antigens, ligands, proteins, drug-like molecules, etc.) find use with the assay particles herein. Different linkers and conjugation techniques (e.g., covalent, affinity, etc.) for the components of the assays find use with the assay particles herein. The disclosure herein contemplates assay particles comprising the full scope of the combinations of components (e.g., luminescent reporters, target-specific interaction moieties, secondary components, linkers, conjugations, mimetics, etc.) on/within the full scope of assay particles (e.g., different matrix materials, porosities, particle size/morphologies, etc.) to produce a diverse array of systems and assays within the scope herein.

Although embodiments herein feature a full range of luminescent (e.g., fluorescent, chemiluminescent, bioluminescent etc.) reporter options. Many embodiments herein utilize two components of a luminescent reporter system (e.g., luminescent reporter and secondary reporter) to produce a luminescent signal that is indicative of an association (e.g., physical association, proximity, etc.) between the two reporters (or components of a reporter). Some preferred embodiments utilize bioluminescence (e.g., primary bioluminescent reporter, bioluminescent structural complementation, BRET, etc.), for example, due to the reduced background noise, enhanced sensitivity, and quantitative accuracy, when compared to other detection formats. Bioluminescence-based reporters are dependent on enzymatic chemical reactions for light generation instead of external light sources. Additionally, bioluminescent reporters provide a broader dynamic range, superior signal stability and are free from issues such as photobleaching and phototoxicity. These benefits make bioluminescent reporters ideal for high-throughput screening and long-term kinetic studies, where precision and reliability are paramount. In certain embodiments, bioluminescence-dependent biosensors possess a biorecognition element that interacts directly with a target analyte, thereby modulating bioluminescent reporter signal to reflect changes in analyte abundance or availability.

Provided herein are luminescence-dependent (e.g., bioluminescence-dependent) biosensors that are contained on/within (e.g., tethered to and/or contained within the interstitial void volume) porous assay particles encoded with a unique/identifiable assay-particle-identifying physical characteristic (e.g., spectral signature). Because the components of an assay particle can be determined based on detection of the assay-particle-identifying physical characteristic, the assay particles herein may be referred to as “encoded” assay particles. The assay readout of two different particles may produce the same luminescent signal in response to two different analytes; however, the assay-particle-identifying physical characteristic allows differentiation of the two particles and assignment of the luminescent signal to the correct analyte. The encoded porous assay particles loaded with bioluminescent biosensors provide modular and discrete sensor units. Each biosensor-loaded assay particle leverages its interstitial void volume (e.g., pore space) to autonomously enrich, detect, and quantify analytes of interest. Employing imaging-based readouts that take advantage of the spatial separation provided by reactions spaces within separate assay particles, each encoded assay particle represents an individual assay. Thus, the change of signal intensity (e.g., gain or loss) of each assay particle represents, for example, the quantity of a target analyte when it engages with the specific biosensor loaded into/onto the assay particle. This approach provides quantitative and independent interrogation of multiple analytes (multiplexing) in complex biological samples and for compound profiling.

Embodiments herein also provide assay particles that serve as individual reaction compartments for detection of analytes. By accommodating fractions of different samples in small compartments, many different assays or samples are accommodated in a single reaction well or chamber and to detect/image the signal generated within the assay particles faster and more sensitively in a single image. This addresses a fundamental problem with luminescence assays, which is the inherently small amount of light that is generated by the reporter system. This leads to long acquisition times, expensive sensors, and has an impact on achievable test and sample throughput.

In some exemplary embodiments, systems herein provide the ability to isolate the reaction space within the interstitial void volume of assay particles by a physical barrier (e.g., by dispersion of the assay particles in non-aqueous media) and to externally trigger reactions within the reaction spaces isolated from other assay particles. In some embodiments, the necessary molecular components for the detection of a specific analyte are provided in a captured or inactive form in/on a spectrally encoded assay particle. Following analyte capture (e.g., by binding) or take-up (e.g., by diffusion into the bead space), the assay particles are transferred into a non-aqueous liquid (e.g., dispersed into non-aqueous medium) to isolate the individual assay particles from the aqueous sample and from one another. Detection/reaction is then activated (e.g., by reagent release into the reactions space, by addition of a luminophore substrate (e.g., in a non-aqueous carrier), etc.).

An exemplary advantage of the technologies herein is in incorporation of luminescence-based (e.g., bioluminescence-based reporter technologies (e.g., primary bioluminescence, bioluminescent structural complementation, BRET) into/onto porous assay particles to spatially confine analyte detection reactions within isolated and differentiable (e.g., via distinct spectral signatures) reaction spaces.

In other exemplary embodiments, fluid communication is maintained between the interstitial void volume of an assay particle (and/or the interstitial void volumes of a population of assay particles) and the sample within which the assay particle(s) reside. In some embodiments, one or more (all) of the steps of an assay are conducted without isolating the interstitial void volume from the sample. An advantage of such embodiments is a reduction in processing steps and the ability to monitor the analytes in a sample in real-time.

In some embodiments, distinct analyte detection reactions (e.g., different target analytes, different target analyte interaction elements, etc.) are performed in separate individual assay particles that are processed in parallel within the same volume (e.g., a well), are measured and deconvoluted simultaneously, thereby increasing the density of information obtained from a single measurement.

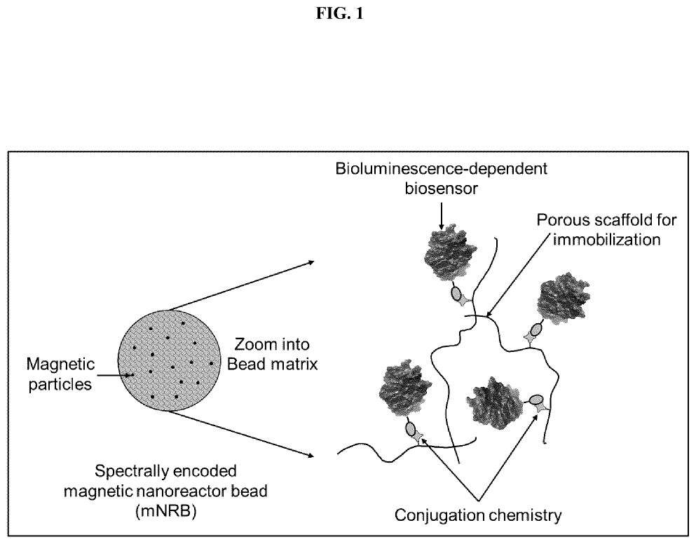

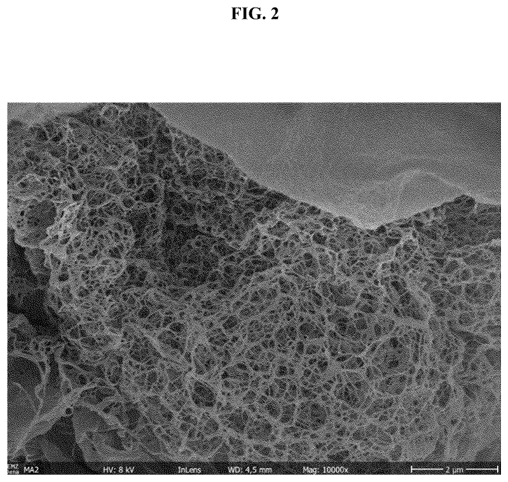

As seen in FIG. 1, in certain embodiments, bioluminescence-based biosensors/reporters are tethered onto hydrogel-based magnetic assay particles with spectral signatures provided by encoded particles. The exemplary assay particle of FIG. 1 does not limit the full scope herein, as assay particles within the scope herein may comprise a spectral signature provided without the presence of encoded particles, reporters may be non-bioluminescent (e.g., fluorescent, chemiluminescent, etc.), assay particles may be non-magnetic, etc. Consistent with FIG. 1, assay particles herein provide a matrix material (see, e.g., FIG. 2), optionally for tethering a reporter and other detection/reaction components, an interstitial void volume to provide a reaction space to contain and/or confine detection/reaction, and pores to provide access to the interstitial void volume by an external sample. In some embodiments, magnetic particles on or within the assay particles (e.g., embedded into the matrix material, within the interstitial void volume, tethered to the assay particle, etc.) allow for bead handling and immobilization during, for example, wash and incubation steps, under both steady and dynamic flow conditions. In certain embodiments, an assay-particle-identifying physical characteristic (e.g., a spectral signature (e.g., provided by encoded particles)) indicates the identity of each assay particle (e.g., identity of target analyte, identity of target-analyte interaction moiety, etc.) but does not directly participate in the reaction/assay. Each assay particle has pores to allow for the free or forced entry of biomolecules, buffers, substrates, dyes, and other chemical compounds. Different sized assay particles and pores can be provided in an application-specific manner.

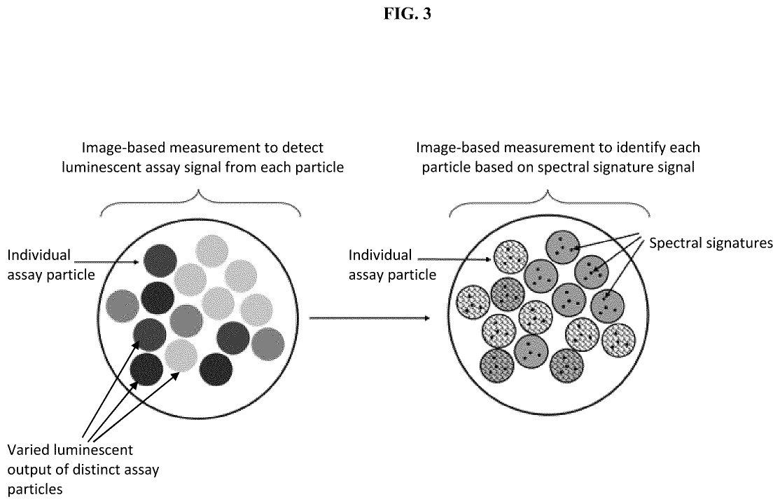

In some embodiments, a population of assay particles is provided. A population of assay particles share certain features (e.g., size, porosity, certain assay components, identity of luminescent reporter, etc.). Within a population of assay particles, multiple different subsets of assay particles may be provided. Assay particles within a subset are substantially identical (e.g., equivalent) to each other but exhibit certain differences with assay particles of different subsets (e.g., different target analyte, different target-analyte interaction moieties, etc.). In some embodiments, distinct assay particles (e.g., assay particles of different subsets within a population) comprise distinct assay-particle-identifying physical characteristics (e.g., size, spectral signatures, etc.). The assay-particle-identifying physical characteristic (e.g., spectral signature) allows for identification of the assay particle and/or subset to which it belongs in an assay-independent manner. In certain embodiments, assay particles of each subset within a population are pre-loaded with distinct target-specific luminescent detection agents (e.g., comprising distinct target-specific interaction moiety), and a luminescence signal from an assay particle within a given subset represents a molecular interaction that is specific to that subset. The luminescence signal from an assay particle within a given subset can be detected. Detection of the assay-particle-identifying physical characteristics of an assay particle provides correlation of the luminescence signal to the subset. In some embodiments, the spectral signature is provided by a differential combination of encoded particles (e.g., fluorescently encoded particles), such as depicted in FIG. 3. A subset-specific combination of encoded particles can be used to identify the assay particles of the subset and differentiate them from other assay particles within the population. Alternatively, the spectral signature may be provided in certain embodiments by a subset-specific combination of fluorophores (or other detectable entities) conjugated (e.g., via DBCO conjugation, click chemistry, etc.) to the matrix material. A unique assay-particle-identifying physical characteristic (e.g., spectral signature) identifies the assay particles of a subset such that all assay particles with the same reaction components and/or intended to measure a specific analyte share spectral signature that is distinct and separate from spectral signatures of assay particles of other subsets (e.g., with other reaction components and/or intended to measure another analyte). In some embodiments, detection/reaction is localized within the boundaries of individual assay particles. As such, a single image of a population of assay particles collectively represents multiple discrete reaction spaces capable of multiple instances of multiple different detection/reaction events, allowing for multiplexing based on spatial separation. This provides for a modular and discrete biosensor platform in which one assay particle represents one assay, and all replicates (of the same assay or of different but parallel assays) are included within one measurement.

In some embodiments, provided herein are assay particles, populations of assay particles, subsets of assay particles, and methods of using such assay particles for the detection and/or quantification of target analytes in a sample. In some embodiments, provided herein are biosensor platforms (e.g., comprising assay particles having a matrix material, interstitial void volume, and assay-particle-identifying physical characteristics (e.g., spectral signature), etc.) that depend on a luminescent reporter (or pair of luminescent reporters) as a readout for detection of a target analyte (e.g., by a target-specific interaction moiety).

In some embodiments, the assay particles are of any suitable size (e.g., 5 μm, 10 μm, 20 μm, 50 μm, 100 μm, 200 μm, 500 μm, or more or ranges therebetween). A particle may preferably be a microparticle having dimensions between 5 μm and 500 μm, more preferably between 10 μm and 250 μm (e.g., 10 μm, 20 μm, 30 μm, 40, μm, 50 μm, 75 μm, 100 μm, 125 μm, 150 μm, 200 μm, 250 μm, and ranges or values therebetween), and even more preferably between 20 μm and 200 μm (e.g., 20 μm, 30 μm, 40, μm, 50 μm, 75 μm, 100 μm, 125 μm, 150 μm, 200 μm, and ranges or values therebetween). In some embodiments, particle size may be used as an assay-particle-identifying physical characteristic (alone or in conjunction with one or more features). For example, a first subset of assay particles of a first size may be provided for analysis of a first analyte, and a second subset of assay particles of a second size may be provided for analysis of a second analyte. Although both subsets produce the same luminescent signal in response to their specific analytes, the particles can be differentiated by the particle size, and therefore the signals can be correlated to the proper analyte. Other assay-particle-identifying physical characteristics are described herein and may be used along with particle size for identifying a particle or a subpopulation thereof.

In some embodiments, the assay particles described herein are beads or microbeads and have a generally spherical, spheroid, oval, or ellipsoidal microparticle (e.g., substantially equal dimensions (e.g., radius). In some embodiments, the assay particles have non-uniform dimensions. In some embodiments, the shape of assay particles may be used as an assay-particle-identifying physical characteristic to differentiate subsets of distinct assay particles.

In some embodiments, the assay particles described herein comprise a matrix material. In some embodiments, the matrix material provides a three-dimensional network that forms the body of the particle support thereof. A suitable matrix material may be any natural, synthetic, or hybrid material capable of forming a porous structure that can receive and contain a sample. In some embodiments, the matrix material comprises a polymer material. In some embodiments, polymer material is biocompatible. The matrix material may comprise any suitable combination of polymer materials to form the body of the particle. For example, the matrix material may comprise one or more natural polymers, modified natural polymers, or synthetic polymers. Suitable natural polymers may include, but are not limited, to agarose, chitosan, alginate, chitin, gelatin, carrageenan, collagen, fucoidan, cellulose, laminaran, fibrin, hyaluronic acid, carboxymethyl cellulose, starch, pectin, keratin, chondroitin sulfate, pullulan, xanthan gum, arabic gum, ghatti gum, guar gum, locust bean gum, tragacanth gum, karaya gum, inulin, and albumin. Suitable synthetic polymers may include, but are not limited to polylactic acid (PLA), poly(lactic-co-glycolic acid) (PLGA), polycaprolactone (PCL), polyethylene glycol (PEG), polyvinyl alcohol (PVA), polymethyl methacrylate (PMMA), polyurethane (PU), poly(D,L-lactic acid) (PDLLA), polyethylene-co-vinyl acetate (EVA), poly(N-isopropylacrylamide) (PNIPAAm), polysiloxane (Silicone), poly(glycolic acid) (PGA), poly(¿-caprolactone-co-lactide) (PCLLA), poly(3-hydroxybutyrate) (PHB), polyvinylidene fluoride (PVDF), poly(alkylene oxides), and citric acid based polymers (e.g., polyoctanediol). In some embodiments, various co-polymers, crosslinked polymers, derivatives of polymers, and any suitable combinations of polymers herein are contemplated for use as matrix material in the assay particles herein.

In some embodiments, pores are provided within the matrix material. In some embodiments, the pores allow a sample (e.g., liquid (e.g., aqueous sample)) to enter the assay particles. In some embodiments, the pores within the network of matrix material provide an interstitial void volume within the particle that is available to be occupied by a sample. The structural parameters of the pores within the matrix, including the pore size, pore interconnectivity/tortuosity and surface area, can affect how substances (e.g., fluid, solutes, analytes, macromolecules, etc.) access, move about, and occupy the particle. In some embodiments, when a sample (e.g., aqueous sample) is introduced to the assay particles herein, the porosity of the assay particles and interstitial void volume allows the assay particles to receive or take up a portion of the sample (e.g., aqueous sample), and, if present, any solutes therein, such as an analyte (e.g., small molecule, peptide, protein, antibody, etc.). In some embodiments, assay particles herein are substantially homogeneous structures, and the pores, matric material, interstitial void volume, and other components therein are distributed substantially uniformly throughout the assay particle. Although matrix material and/or pore systems may be locally irregular (e.g., not adopting a uniform repeating pattern), they are substantially homogeneous across the assay particles.

In some embodiments, an assay particle is present as (or capable of forming) a hydrogel, wherein the matrix material forms a three-dimensional polymeric structure that is insoluble or minimally soluble in aqueous solution, but is capable of receiving a sample (e.g., aqueous solution and solutes therein (e.g., an analyte) and retaining it within the interstitial void volume. In certain embodiments, the matrix material is cross-linked via covalent, ionic, or hydrogen bonds to create a three-dimensional open-lattice structure that is capable of holding an aqueous solution to form a gel particle. In some embodiments, the assay particle, matrix material, and/or a hydrogel thereof is liquefiable, for example, (1) upon the application of heat, light, and enzyme, or (2) a change of pH, redox potential, ionic strength, temperature, magnetic field, or electromagnetic radiation. In some embodiments, an assay particle and/or the matrix material thereof is capable existing in two different states, one state being a solid state or semi-solid state, the other state being a liquid state. In one embodiment, in the solid state or semi-solid state, the matrix material (e.g., a gel-forming material) is present in the form of a gel, which forms the assay particle. Effectively, in this state, the assay particle is reinforced by a matrix formed by the gel-forming agent/gel. In such embodiments, the matrix material defines the surface and the void volume of the microparticle. In a further embodiment, the matrix material (e.g., a gel-forming material) may be transformed from the solid/semi-solid state into a liquid state upon the application of an appropriate stimulus. As addressed above, such stimulus may be, for example, the application of heat or light or it may involve a change of pH, redox potential, ionic strength, temperature, magnetic field, or electromagnetic radiation. Alternatively, such external stimulus may also be the exposure to an enzyme (which, for example, may digest the matrix material (or crosslinks therein)).

In some embodiments, an assay particle is present as a solid porous homogeneous assay particle with an interconnected interstitial void volume.

In certain embodiments, an assay particle herein is prefabricated. In some embodiments, the assay particles are generated prior to use in an assay or reaction mixture. In some embodiments, one or more of the components of an assay particle (e.g., matrix material, target-specific luminescent detection agent, magnetic particles, spectral signature, etc.) are present and arranged within the assay particle in substantially the form (e.g., porous, having an interstitial void volume, containing reagents for analyte detection, etc.) for introduction of a sample or reaction mixture and/or for use in an assay. In some embodiments, the assay particles herein are not generated during use in an assay and/or in the sample with which they will be used. In some embodiments, a prefabricated assay particle is added directly to a sample (or vice versa). In some embodiments, one or more additional assay reagents (e.g., luminescent substrate; non-aqueous dispersion agent, etc.) may be added to an assay particle for performing an assay (e.g., before or after introducing a sample). In some embodiments, one or more post-fabrication steps (e.g., liquefication, rehydration, dispersal in a non-aqueous medium, etc.) may be conducted (e.g., within an analyte-containing sample) with a prefabricated assay particle that alter its structure, composition, component elements, etc.

In some embodiments, any suitable methods of fabricating assay particles, for example, using the materials and techniques described herein and understood in the field may find use in fabricating assay particles consistent with the embodiments described herein. An exemplary fabrication process is described in Example 1 of EP3839509A1 (incorporated by reference in its entirety), the materials and methods of which may find use in fabrication of assay particles herein. Other processes for fabrication of the assay particles may find use in embodiments herein.

Exemplary techniques can be used to create assay particles within the scope herein having different properties, such as size, shape, porosity, matrix material, etc. For example, emulsion polymerization has been widely used to produce polymer particles. In single emulsion, a polymer is dissolved in a solvent and then emulsified in an aqueous phase, forming either oil-in-water (O/W) or water-in-oil (W/O) emulsions; the solvent is evaporated, causing the polymer to precipitate into microparticles. In double emulsion, a water phase is encapsulated by an organic phase and then emulsified again in water. Another particle fabrication technique is solvent evaporation, in which a polymer is dissolved in a volatile organic solvent and then the solvent is evaporated to form the particles. The polymer solution may be emulsified in an aqueous phase (for single emulsion) or water (for double emulsion) to form droplets, and these droplets solidify as the solvent evaporates, leaving behind solid particles. In spray drying, a polymer solution or suspension is sprayed into a heated chamber causing the solvent to rapidly evaporate, leading to the formation of particles. In coacervation (or phase separation), a polymer is dissolved in a solvent, and a coacervate phase is induced by adding a nonsolvent (e.g., gelatin, arabic gum, polyelectrolyte systems, etc.) or adjusting conditions such as pH or temperature. The polymer precipitates out of the solution in the form of particles. In electrospinning, a polymer solution is electrostatically drawn through a charged needle, forming the particles. By adjusting parameters such as solution viscosity and electric field, the size and/or morphology of the particles can be controlled. In melt exclusion, a polymer is melted, and then extruded through a nozzle or die; upon cooling, the polymer solidifies into particles. In some embodiments, a polymer is dissolved in a supercritical fluid, and then the pressure is reduced, or temperature is altered, to precipitate out the polymer as solid particles. In hydrodynamic focusing, a polymer solution is forced through a narrow microchannel where it is rapidly mixed with a non-solvent; the polymer solution and non-solvent are injected into a microfluidic device under high pressure, causing the polymer to precipitate and form particles. In freeze-drying, a polymer solution is frozen and then subjected to a vacuum, which removes the solvent by sublimation, leaving behind solid polymer particles. Each of the above techniques can be tailored and/or combined according to knowledge in the field to produce particles for use in embodiments herein (e.g., as assay particles) with specific characteristics such as size, shape, surface properties, porosity, etc. The choice of technique(s) may depend on the desired properties of the assay particles, and the nature of the matrix material to be used.

As noted throughout, assay particles in some embodiments herein are porous and/or comprise an interstitial void volume within and/or between portions of the matrix material. The pores and/or void within the particles can be achieved by any suitable fabrication techniques. In some embodiments, the characteristics of the matrix material inherently produces pores and/or an interstitial void volume upon formation of a particle therefrom. For example, polymers capable of forming hydrogels may form particles that inherently comprise a volume capable of uptake of a sample (and components therein) into a volume within the particle. In some embodiments, the volume/mass of matrix material and the size of the particles determines the porosity and/or interstitial void volume within particles (e.g., a greater ratio of particle size to matrix material volume will result in a greater porosity and/or interstitial void volume). In other embodiments, fabrication steps are taken to produce the desired degree of porosity and/or interstitial void volume.

In some embodiments, a porogen is used to fabricate porous assay particles and/or assay particles comprising an interstitial void volume. In some embodiments, particles are formed from a matrix material by any suitable process in the presence of a suitable porogen. After formation of the particles, the porogen is removed by, for example, melting, dissolving, degrading, etc., producing pores and/or interstitial void volume by the space previously occupied by the porogen within the particle. The porogen essentially reserves space in the particles during fabrication of the particles; once the porogen diffuses, dissolves or degrades, pores and/or an interstitial void volume remains. In this way, porogens provide latent pores. A porogen may be a gas (e.g., carbon dioxide, nitrogen, or other inert gas), liquid (e.g., water), or solid. Porogens may be water soluble such as salts, sugars (e.g., sugar alcohols), polysaccharides (e.g., dextran (poly(dextrose)), water soluble small molecules, etc. Porogens can also be natural or synthetic polymers, oligomers, or monomers that are water soluble or degrade under conditions in which the matrix material does not. Exemplary polymer porogens include polyethylene glycol, poly(vinylpyrollidone), pullulan, poly(glycolide), poly(lactide), poly(lactide-co-glycolide), other polyesters, and starches.

The assay particles herein provide a reaction space for, for example, binding an analyte, detecting analyte, isolating a portion of a sample, producing a luminescent signal, containing assay reagents, etc. In certain embodiments, the reaction space is the interstitial void volume defined by the space within the maximal dimensions of the assay particle and not occupied by the matrix material. Depending upon the composition of the assay particle, 25-99% of the volume of the assay particle (e.g., 25%, 30%, 35%, 40%, 45%, 50%, 55%, 60%, 65%, 70%, 75%, 80%, 85%, 90%, 95%, 96%, 97%, 98%, 99%, or ranges therebetween) is interstitial void volume and can be provided as a reaction space within the assay particle.

In some embodiments, an assay particle herein can be contacted with an external isolation reagent (e.g., oil, emulsifier, etc.) resulting in the interstitial void volume and contents therein (e.g., sample, target-specific luminescent detection agent, analyte, etc.) being isolated from the environment (e.g., reaction mixture, sample, etc.) external to the assay particle. Contacting an assay particle with such an external isolation reagent (e.g., oil, emulsifier, etc.) results in an isolated reaction space being formed within the assay particle. Reagents, sample, analyte, etc., cannot enter or exit the isolated reaction space and cannot move between the isolated reaction spaces of two or more assay particles (e.g., without a suitable carrier). In some embodiments, contacting an assay particle with a suitable external isolation reagent (e.g., oil, emulsifier, etc.) under proper conditions produces an isolation barrier around the periphery of the assay particle and produces an isolated reaction space.

External isolation reagents for generating an isolation barrier around assay particles and/or producing an isolated reaction space are selected based on the composition of the assay particles (e.g., matrix material), the sample or reaction mixture composition, the reaction conditions, and other factors understood in the field. In some embodiments, an external isolation reagent may include one or more (e.g., 2, 3, 4, or more) oils, non-polar solvents, non-aqueous solutions, immiscible fluids, etc. Exemplary oils, non-polar solvents, non-aqueous solutions, immiscible fluids, etc., include, but are not limited to decane (e g., tetradecane or hexadecane), fluorocarbon or fluorinated oils (e.g., Perfluoropolyether (PFPE) Oils, Fluorosilicone Oils, Perfluorooctyltrichlorosilane (PFOTS)-based Oils, perfluorocyclopropylmethyl (PFCM) Oils, Fluorinated Polyethylene Oils, Perfluorinated Alkyl Ester Oils, etc.), silicone oils or copolymers (e.g., poly(dimethylsiloxane), methylphenylsiloxane copolymers, silicone-polyether copolymers, fluorosilicone copolymers, silicone-urethane copolymers, silicone-polycarbonate copolymers, dimethicone copolyols (silicone-glycol copolymers), phenylmethylsiloxane copolymers, etc.), heptane, carbonate oils (e.g., diethylhexyl carbonate), mineral oil, isopropyl palmitate, paraffin oils, castor oil, coconut oil, etc., or combinations thereof.

In some embodiments, an external isolation reagent may include one or more (e.g., 2, 3, 4, or more) emulsifiers, surfactants, detergents, etc. The concentration of an emulsifier, surfactant, detergent, etc., may be about 0.05% to about 20. % (e.g., 0.05%, 0.1%, 0.2%, 0.5%, 1%, 2%, 5%, 10%, 20%, or ranges therebetween). In a preferred embodiment, the concentration of the emulsifier, surfactant, detergent, etc., is between 1% to about 25% (e.g., 1%, 2%, 5%, 10%, 15%, 20%, 25%, and values or ranges therebetween (e.g., 5-20%). Suitable surfactants may be anionic, cationic, amphoteric, or nonionic surface-active agents. Suitable anionic surfactants include, but are not limited to, those containing carboxylate, sulfonate, and sulfate ions. Examples of anionic surfactants include sodium, potassium, ammonium of long chain alkyl sulfonates, and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)sulfosuccinate; and alkyl sulfates, such as sodium lauryl sulfate. Cationic surfactants include, but are not limited to, quaternary ammonium compounds, such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine. Suitable detergents include, for example, pluronic F-127, Tween 20, Tween 80, Triton X-100, sodium dodecyl sulfate (SDS), cetyl trimethyl-ammonium bromide (CTAB), digitonin, etc. Suitable emulsifiers include, for example, lecithin, saponins, ammonium phosphatide (E442), polyglycerol polyricinoleate (E476), diacetyl tartaric acid ester of monoglyceride, DOWSIL ES-5300 Formulation Aid (a non-diluted and low viscosity silicone surfactant designed to produce stable water-in-silicone emulsions and water-in-oil emulsions), etc. In some embodiments, particularly with silicone oils and copolymers (although not limited for use therewith), the following emulsifiers may be used to create water-in-oil emulsions to disperse assay particles (e.g., create an isolation barrier): cetyl PEG/PPG-10/1 dimethicone (found in products like PURESIL™ BALANCE E6 and BELSIL® WO 3700), dimethicone and caprylyl dimethicone ethoxy glucoside (used in BELSIL® WO 5000), PEG-10 dimethicone (present in BRB 6341 and Cosmethicone® SF-9336), dimethicone/PEG-10/15 crosspolymer (found in Andisil® CP 2050), dimethicone/polyglycerin-3 crosspolymer (used in Andisil® CP 3020), lauryl PEG-9 polydimethylsiloxyethyl dimethicone (found in Cosmethicone® SF-9335), cetyl diglyceryl tris (trimethylsiloxy)silylethyl dimethicone (DOWSIL™ ES-5600), dimethicone, and PEG/PPG-18/18 dimethicone (used in DOWSIL™ ES-5226 DM), etc. In some embodiments, particularly with mineral oil (although not limited for use therewith), the following emulsifiers may be used to create water-in-oil emulsions to disperse assay particles (e.g., create an isolation barrier): sorbitan monooleate (Span 80) (a non-ionic surfactant commonly used for its excellent emulsifying properties), polyglycerol polyricinoleate (PGPR) (commonly used in food and cosmetic applications to stabilize emulsions), lecithin (a natural emulsifier derived from soy or egg yolk, widely used in various industries), oleyl alcohol polyglycol ether (Emulsogen M) (excellent emulsifying properties with mineral oils), sorbitan sesquioleate (a non-ionic surfactant that helps stabilize water-in-oil emulsions), PEG-30 dipolyhydroxystearate (a versatile emulsifier that helps stabilize mineral oil in water-in-oil systems), cetyl PEG/PPG-10/1 dimethicone (a silicone-based emulsifier for stabilizing mineral oil in water-in-oil emulsions), etc. In some embodiments, particularly with fluorinated oils/copolymers (although not limited for use therewith), the following emulsifiers may be used to create water-in-oil emulsions to disperse assay particles (e.g., create an isolation barrier): perfluoropolyether (PFPE) surfactants including Tri- and Di-block copolymers (specifically designed for use with fluorinated oils due to their compatibility and stability), Zonyl® FSN (a non-ionic fluorosurfactant that helps stabilize water-in-oil emulsions with fluorinated oils), Krytox® surfactants (known for their excellent emulsifying properties with fluorinated oils), fluorosilicone surfactants (effective in stabilizing water-in-oil emulsions with fluorinated oils), fluorinated nanoparticles, etc.

In some embodiments, additives may be added to the aqueous phase (e.g., aqueous sample comprising the assay particles) for dispersion into the oil emulsion (e.g., prior to addition of oil and/or emulsifier). Exemplary additives include be surfactants, salts, sugars, proteins, polymers, and some solid particles.

In some embodiments, surfactants facilitate exchange between water in oil emulsions carrying the ligand and the suspended beads by reducing the surface tension between the water and oil phases. Anionic surfactants, such as sodium dodecyl sulfate (SDS) and sodium stearoyl lactylate, have a negatively charged hydrophilic head. Cationic surfactants, such as cetyltrimethylammonium bromide (CTAB) and benzalkonium chloride, have a positively charged hydrophilic head. Non-ionic surfactants, such as Tween 80 (polysorbate 80), Span 80 (sorbitan monooleate), and PEG-40 hydrogenated castor oil, do not carry any charge. Amphoteric surfactants, such as lecithin and cocamidopropyl betaine, can carry both positive and negative charges depending on the pH. Any of the above surfactants, others described herein, or surfactants understood in the field may find use in embodiments herein.

Salts influence the stability of emulsions by affecting the ionic strength and the electrostatic interactions between aqueous droplets. For instance, sodium chloride (NaCl) is commonly used to adjust the ionic strength and stabilize emulsions by influencing electrostatic interactions between droplets. Calcium chloride (CaCl₂)) is used to affect emulsion stability. In some embodiments, salts (e.g., sodium chloride, calcium chloride, magnesium sulfate, Potassium Chloride, Sodium Carbonate, Sodium Citrate, Calcium Lactate, Ammonium Sulfate, Sodium Bicarbonate, Sodium Phosphate, Zinc Sulfate, Sodium Sulfate, Sodium Stearate, Potassium Carbonate, etc.) find use in formulation of emulsions herein.

In some embodiments, emulsions herein (e.g., comprising assay particles or other components of the assays herein (e.g., luminescent substrates, ligands, etc.) comprise large molecular additives, such as sugars, proteins, polymers, solid particles, etc. Sugars can impact the viscosity and osmotic pressure within the emulsion, thereby affecting its stability, and thus allow for transfer between emulsions (e.g., addition of a ligand or substate within one emulsion to an assay particle within a second emulsion). For instance, sucrose increases the viscosity of the aqueous liquid, which has an effect on transfer ability. Glucose also can affect the osmotic pressure and viscosity, contributing to emulsion quality. Similar effects can be achieved with polymers like xanthan gum or guar gum. Proteins can act as emulsifiers and stabilizers by adsorbing at the oil-water interface and forming a protective layer around the droplets. Examples of such proteins are casein or BSA, that can adsorb at the oil-water interface and form a protective layer around droplets, and Whey Protein, which acts as an emulsifier and stabilizer. Some solid particles, such as clay platelets or asphaltenes, can also stabilize emulsions by forming a rigid film around the droplets. Another example would be asphaltenes, large polyaromatic compounds found in crude oil that can stabilize emulsions by forming a protective layer around droplets.

In some embodiments, buffers are used to affect/control the pH of emulsions, dispersions, reaction mixtures, samples, etc., described herein. For example, several pH buffers can be used to affect the stability of water-in-oil emulsions, such as citrate buffer (acidic to neutral range of pH 3-6), acetate buffer (effective in the acidic range around pH 4-5), phosphate buffer (commonly used in the neutral pH range), Tris buffer (effective in the alkaline pH range around pH 8-9), etc. Buffers can stabilize emulsions by influencing the electrostatic interactions and viscosity of the continuous phase. Exemplary buffers that may find use in embodiments herein include borate buffer, HEPES buffer, MES buffer, glycine buffer, and others.

Components of external isolation reagents herein may be classified in one or more of the categories of oils, emulsifiers, surfactants, detergents, and additives. In some embodiments, an external isolation reagent comprises an oil and an emulsifier. In some embodiments, an external isolation reagent further comprises one or more surfactants, detergents, additives, etc. In some embodiments, an external isolation reagent comprises a silicone oil and a silicone surfactant (e.g., DOWSIL ES-5300 Formulation Aid). In some embodiments, the emulsifier (e.g., silicone surfactant) is present at a concentration of 2-20% (e.g., 2%, 5%, 10%, 15%, 20%, or ranges therebetween).

In some embodiments, an isolation barrier is generated around an assay particle, thereby producing an isolated reaction space, by establishing a physical barrier around the assay particle and/or otherwise mechanically separating the assay particle from other assay particles and/or the sample.

In some embodiments, contacting an assay particle (or population of assay particles) with an external isolation reagent results in dispersion of the assay particle(s) within the aqueous sample or reaction mixture within which they reside, isolation of the assay particle(s) from the aqueous sample or reaction mixture, and formation of an isolated reaction space within the assay particle(s). As noted above, components (e.g., solutes, small molecules, macromolecules, analytes, reagents, etc.) residing in the aqueous environment within the isolated reaction space cannot exit the assay particle(s), and components in the aqueous environment outside the assay particle(s) cannot enter the assay particle(s). However, components (e.g., solutes, small molecules, macromolecules, analytes, reagents, etc.) formulated with a proper carrier (e.g., oil, emulsifier, etc.) can be added externally to the particle(s) and can cross the isolation barrier to deliver the components to the isolated reaction space. Isolating assay particles of a population and subsequently adding reagents to the isolated assay particles has a number of potential benefits to particular assays that can be performed with the assay particles. For example, once isolated reaction spaces have been generated within the assay particles, tethered reagents (e.g., target-specific luminescent detection agents, secondary detection agents, etc.) can be untethered within the isolated reaction space without exiting the assay particle or moving between assay particles. Formulating additional components in a carrier (e.g., oil, emulsifier, etc.) that can cross the isolation barrier allows additional assay reagents (e.g., luminophore substrates) to be added to the isolated reaction spaces, for example, after untethering of reagents within the assay particles. In some embodiments, components (e.g., ligands, substrates, reactants, control targets, etc.) to be added to isolated assay particles (e.g., dispersed within a non-aqueous medium), particularly hydrophilic components, are formulated in a suitable carrier for introduction into the isolated reaction spaces. In some embodiments, the carrier formulation is identical to the non-aqueous medium used to disperse the assay particles. In other embodiments, the carrier is formulated according to the characteristics (e.g., hydrophilicity/hydrophobicity, solubility, size, etc.) of the component(s) to be added. In particular embodiments, a carrier formulation is selected based upon both the non-aqueous medium used to disperse the assay particles and the characteristics of the component(s) to be added.

In some embodiments, carrier formulations may include one or more (e.g., 2, 3, 4, or more) oils, non-polar solvents, non-aqueous solutions, immiscible fluids, etc. Exemplary oils, non-polar solvents, non-aqueous solutions, immiscible fluids, etc., include, but are not limited to decane (e g., tetradecane or hexadecane), fluorocarbon or fluorinated oils (e.g., Perfluoropolyether (PFPE) Oils, Fluorosilicone Oils, Perfluorooctyltrichlorosilane (PFOTS)-based Oils, perfluorocyclopropylmethyl (PFCM) Oils, Fluorinated Polyethylene Oils, Perfluorinated Alkyl Ester Oils, etc.), silicone oils or copolymers (e.g., poly(dimethylsiloxane), methylphenylsiloxane copolymers, silicone-polyether copolymers, fluorosilicone copolymers, silicone-urethane copolymers, silicone-polycarbonate copolymers, dimethicone copolyols (silicone-glycol copolymers), phenylmethylsiloxane copolymers, etc.), heptane, carbonate oils (e.g., diethylhexyl carbonate), mineral oil, isopropyl palmitate, paraffin oils, castor oil, coconut oil, etc., or combinations thereof.

In some embodiments, carrier formulations may include one or more (e.g., 2, 3, 4, or more) emulsifiers, surfactants, detergents, etc. The concentration of an emulsifier, surfactant, detergent, etc., may be about 0.05% to about 20. % (e.g., 0.05%, 0.1%, 0.2%, 0.5%, 1%, 2%, 5%, 10%, 20%, or ranges therebetween). In a preferred embodiment, the concentration of the emulsifier, surfactant, detergent, etc., in a carrier formulation is between 1% to about 25% (e.g., 1%, 2%, 5%, 10%, 15%, 20%, 25%, and values or ranges therebetween (e.g., 5-20%)). Suitable surfactants for a carrier formulation may be anionic, cationic, amphoteric, or nonionic surface-active agents. Suitable anionic surfactants for a carrier formulation include, but are not limited to, those containing carboxylate, sulfonate, and sulfate ions. Examples of anionic surfactants include sodium, potassium, ammonium of long chain alkyl sulfonates, and alkyl aryl sulfonates, such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)sulfosuccinate; and alkyl sulfates such as sodium lauryl sulfate. Cationic surfactants include, but are not limited to, quaternary ammonium compounds, such as benzalkonium chloride, benzethonium chloride, cetrimonium bromide, stearyl dimethylbenzyl ammonium chloride, polyoxyethylene, and coconut amine. Examples of nonionic surfactants for a carrier formulation include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants for a carrier formulation include sodium N-dodecyl-β-alanine, sodium N-lauryl-β-iminodipropionate, myristoamphoacetate, lauryl betaine and lauryl sulfobetaine. Suitable detergents for a carrier formulation include, for example, pluronic F-127, Tween 20, Tween 80, Triton X-100, sodium dodecyl sulfate (SDS), cetyl trimethyl-ammonium bromide (CTAB), digitonin, etc. Suitable emulsifiers include, for example, lecithin, saponins, ammonium phosphatide (E442), polyglycerol polyricinoleate (E476), diacetyl tartaric acid ester of monoglyceride, DOWSIL ES-5300 Formulation Aid, etc. In some embodiments, particularly with silicone oils and copolymers (although not limited for use therewith), the following emulsifiers may be used in carrier formulations: cetyl PEG/PPG-10/1 dimethicone, dimethicone and caprylyl dimethicone ethoxy glucoside, PEG-10 dimethicone, dimethicone/PEG-10/15 crosspolymer, dimethicone/polyglycerin-3 crosspolymer, lauryl PEG-9 polydimethylsiloxyethyl dimethicone, cetyl diglyceryl tris (trimethylsiloxy)silylethyl dimethicone (DOWSIL™ ES-5600), dimethicone, and PEG/PPG-18/18 dimethicone, etc. In some embodiments, particularly with mineral oil (although not limited for use therewith), the following emulsifiers may be used in carrier formulations: sorbitan monooleate (Span 80), polyglycerol polyricinoleate (PGPR), lecithin, oleyl alcohol polyglycol ether (Emulsogen M), sorbitan sesquioleate, PEG-30 dipolyhydroxystearate, cetyl PEG/PPG-10/1 dimethicone, etc. In some embodiments, particularly with fluorinated oils/copolymers (although not limited for use therewith), the following emulsifiers may be used in carrier formulations: perfluoropolyether (PFPE) surfactants including Tri- and Di-block copolymers, Zonyl® FSN, Krytox® surfactants, fluorosilicone surfactants, fluorinated nanoparticles, etc.

In some embodiments, additives may be added to the aqueous phase (e.g., aqueous sample comprising the components to be added (e.g., substrate, ligand, etc.)) for formulation into the carrier. Exemplary additives for use in formulating a carrier include be surfactants, salts, sugars, proteins, polymers, and some solid particles.

In some embodiments, surfactants may be used in carrier formulation, for example, to reduce the surface tension. Suitable surfactants for carrier formulations herein may include anionic surfactants (e.g., sodium dodecyl sulfate (SDS) and sodium stearoyl lactylate), cationic surfactants (e.g., cetyltrimethylammonium bromide (CTAB) and benzalkonium chloride), non-ionic surfactants (e.g., Tween 80 (polysorbate 80), Span 80 (sorbitan monooleate), and PEG-40 hydrogenated castor oil), and/or amphoteric surfactants (e.g., lecithin and cocamidopropyl betaine).

In some embodiments, one or more salts (e.g., sodium chloride, calcium chloride, magnesium sulfate, Potassium Chloride, Sodium Carbonate, Sodium Citrate, Calcium Lactate, Ammonium Sulfate, Sodium Bicarbonate, Sodium Phosphate, Zinc Sulfate, Sodium Sulfate, Sodium Stearate, Potassium Carbonate, etc.) find use in formulation of carriers herein.

In some embodiments, carrier formulations may comprise large molecular additives, such as sugars, proteins, polymers, solid particles, etc.

In some embodiments, carrier formulations may comprise buffers, such as citrate buffer, acetate buffer, phosphate buffer, Tris buffer, borate buffer, HEPES buffer, MES buffer, glycine buffer, and others.

The assay particles herein can also be used for analyte detection/quantification or for other reactions in aqueous conditions, and do not require non-aqueous environments, such as an oil emulsion, for partitioning. In some embodiments, the assay particles are not partitioned or encapsulated, and the interstitial void volume is not isolated. In some embodiments, a reaction space is provided within the assay particles (e.g., the interstitial void volume) that is in fluid communication with the external sample. This allows the assay particles herein to be directly compatible with aqueous samples, such as tissue-culture media, allowing for sampling of analytes without additional sample preparation. In some embodiments, conducting analyte detection without particle emulsion and/or dispersion simplifies assay protocols, making the platform more robust and user-friendly for various settings, including point-of-care testing and field applications. In some embodiments, assays can be conducted that measure real-time changes to the sample conditions, as changes to external sample conditions result in changes to sample conditions withing the interstitial void volume.

In some embodiments, the assay particles herein comprise one or more magnetic particles. The magnetic particles may be contained within the interstitial void volume, tethered to the matrix material, and/or tethered to the exterior of the assay particle. In some embodiments, application of an external magnetic field to assay particles comprising one or more magnetic particles allows for isolation, sorting, collection, purification, washing, and other manipulations of the assay particles. In some embodiments, the one or more magnetic particles are selected from superparamagnetic particles, paramagnetic particles, diamagnetic particles, ferromagnetic particles, and combinations thereof. The one or more magnetic particles can have diameters that are less than or equal to approximately 0.5 μm, e.g., less than or equal to approximately 0.1 μm (e.g., 5 nm, 10 nm, 20 nm, 50 nm, 100 nm, etc.).

Incorporating magnetic particles into the assay particles provides precise control over their immobilization under both steady and dynamic states. This magnetic manipulation allows for easy handling, separation, and concentration of the assay particles, facilitating streamlined workflows in various applications, such as sample preparation, washing steps, and analyte detection. The ability to control the position and movement of the assay particles enhances the overall flexibility and efficiency of the platform.

In some embodiments, assay particles herein comprise an assay-particle-identifying physical characteristic (e.g., spectral signature) that can be used to identify the assay particles according to their specific composition (e.g., target-specific moiety, secondary detection agent, etc.). In some embodiments, assay particles specific for different analytes may be provided within a population. Because these different particles utilize the same luminescent reporter to produce signals in response to the different analytes, the reporter signal cannot be directly correlated to the analyte. Instead, each particle produces a signal from identical luminescent reporters, and then the particles are identified based on the known assay-particle-identifying physical characteristic of the particle (and the corresponding analyte is identified as well). The inclusion of a assay-particle-identifying physical characteristic for the identification of particle subsets within a population of particles, allows for multiplex analysis of different analytes using the same luminescent reporter for each in the same sample.

An assay-particle-identifying physical characteristic may comprise a single feature of an assay particle (size, shape, spectral coding, etc.). In other embodiments, an assay-particle-identifying physical characteristic may be the combination of multiple features of a particles. For example, a first particle (or subpopulation of particles) may be of size A and non-fluorescently encoded, a second particle (or subpopulation of particles) may be of size B and non-fluorescently encoded, a third particle (or subpopulation of particles) may be of size A and fluorescently encoded with fluorophore X, a fourth particle (or subpopulation of particles) may be of size A and fluorescently encoded with fluorophore Y, a fifth particle (or subpopulation of particles) may be of size B and fluorescently encoded with fluorophore X, a sixth particle (or subpopulation of particles) may be of size B and fluorescently encoded with fluorophore Y. Each particle (or subpopulation of particles) has an assay-particle-identifying physical characteristic that comprises the size of the particle and the fluorescent encoding of the particle. Signal from a luminescent reporter emanating from a particle of size A and fluorescently encoded with fluorophore Y can be assigned as being the result of the analyte of the detection agent for the fourth assay particle (or subpopulation of particles). The luminescent signal from each particle in a population can be assigned to the correct assay particle subset by the corresponding assay-particle-identifying physical characteristic.

In some embodiments, an assay-particle-identifying physical characteristic comprises the size, shape, or dimensions of the assay particle. In some embodiments, an assay-particle-identifying physical characteristic comprises a detectable signal that is emitted by the assay particle or a component thereof. In some embodiments, an assay-particle-identifying physical characteristic comprises the spectral signature of an assay particle (detectable by any suitable detection technology). An identifiable spectral signature for a particle or subpopulation thereof may be the result of inclusion of one or more specialty-encoded molecular constituents of the assay particle.

In some embodiments, an assay particle is encoded with a spectral signature that identifies the molecular constituents of the assay particle. In some embodiments, a subset of assay particles (e.g., having the same target-specific moiety, secondary detection agent, etc.) within a population of assay particles (e.g., equivalent assay particles) have the same spectral signature, and therefore members of the subset can be identified by recognition of the spectral signature. A population of assay particles (e.g., equivalent assay particles) may comprise multiple distinct subsets of the assay particles (e.g., each assay particle within a subset having the same target-specific moiety, secondary detection agent, etc., but each subset having different target-specific moiety, secondary detection agent, etc., from the other subset(s)), and the assay particles of each subset have spectral signatures that are unique to their respective subset. In some embodiments, recognition of the spectral signature of an assay particle allows the assay particle to be identified as belonging to a specific subset. Analysis of the spectral signatures of a population of assay particles (e.g., equivalent assay particles) allows the individual assay particles within the population to be assigned to their respective subsets.

A spectral signature may be any detectable signature that is detectable and distinguishable from the luminescent output of any detection agents (e.g., target specific luminescent detection agent, secondary detection agents, etc.) contained within the assay particle and/or used in the detection of analytes. In some embodiments, the detectable signature signal is a fluorescent signal, a colorimetric signal, a Raman signal, an infrared signal, a chemiluminescent signal, or a bioluminescent signal. In some embodiments, the spectral signatures of distinct assay particles and/or subsets can be differentiated by the nature of the signature signal (e.g., fluorescence vs. chemiluminescence), the wavelength of the signature signal (e.g., a bioluminescent or fluorescent signal at 300 nm vs 600 nm), the brightness of the signature signal (e.g., qualitatively low vs medium vs high signal intensity, or a combination thereof.

In some embodiments, the assay particles of each subset are encoded with a physical label or set of labels that is/are attached to, contained within, or otherwise associated with the assay particles of the respective subset. The physical label or set of labels of an assay particle, or subset of assay particles, is the spectral signature of the assay particle or subset. A detectable signature signal produced by the physical label of one subset is differentiable from the signature signal produced by the physical label of other subsets. In some embodiments, the physical label(s) that produce the detectable signature signal are not involved with target analyte detection/quantification and/or other assay-related functions of the assay particle; rather, the physical label(s) that produce the detectable signature signal are solely used for identification of the assay particle (e.g., identifying the subset to which they belong, identifying the assay components of the particle, identifying the target analyte to which the assay particle is specific, etc.). The physical label may be a fluorophore, quantum dot, Raman detectable label, chromophore, luciferase, or any suitable entity capable of emitting a signal that can be correlated to the identity of the assay particle. In some embodiments, a physical label (or labels) is conjugated or otherwise tethered to the matrix material by any suitable chemistry and/or linker. For example, click chemistry amenable dyes, such as fluorophores conjugated to dibenzocyclooctyne (DBCO), may be used to tether a physical label to the assay particle. In other embodiments, the matrix material itself may provide a detectable signature signal (e.g., spectrally-coded (e.g., fluorescent) polymers such as chitosan integrated into the matrix material).

In some embodiments, the assay particles of each subset produce a signature signal that can be used to identify the assay particles of the subset and differentiate the assay particles of the subset from assay particles (e.g., equivalent assay particles) of other subsets based on the inherent characteristics of the components of the assay particles. In such embodiments, the assay particles exhibit a spectral signature without inclusion of a physical label for the specific purpose of generating the signature signal. For example, particles from distinct subsets may produce detectably distinct Raman spectra based on the components of the assay particles, without the inclusion of a label or other component for the purpose of generating the distinct Raman signal.

In some embodiments, combinations of different features (physical features, spectral signatures, etc.) are used in combination to produce an assay-particle-identifying physical characteristic.

Although multiple (2, 3, 4, 5, 10, 15, 20, 25, 30, 40, 50, or more) subsets of a population of assay particles may each be provided for the detection or quantification of separate target analytes, the luminescent signal produced by the assay particles of each subset is the same (e.g., produced by identical luminescent reporters), and therefore the target analyte being detected/quantified cannot be determined based on the detection of the luminescent reporter signal. However, because the assay-particle-identifying physical characteristic (e.g., physical characteristic, spectral signature, etc.) of each subset is unique to that subset, the identity of a target analyte responsible for producing a luminescent signal (e.g., an otherwise indistinguishable signal) in an assay particle can be determined by correlating the assay-particle-identifying physical characteristic (e.g., physical characteristic, spectral signature, etc.) of the assay particle to the luminescent signal. The luminescent signal identifies the presence/amount of an analyte present in the sample, and the assay-particle-identifying physical characteristic (e.g., physical characteristic, spectral signature, etc.) of the assay particles is used to determine the identity of the target analyte (and/or assay particle).

In some embodiments, assay particles provided herein comprise a target-specific luminescent detection agent. The target-specific luminescent detection agent is capable of (1) interacting with (e.g., binding to) the target analyte and (2) producing a luminescent signal (e.g., alone or in combination with one or more other components). In some embodiments, interaction of the target-specific luminescent detection agent with a target analyte alters (e.g., increases, decreases) the luminescent signal produced by the target-specific luminescent detection agent. Measurement of the luminescent signal and/or monitoring alterations of the luminescent signal over time provides a readout for interactions (e.g., binding events) between the target-specific luminescent detection agent and the target analyte. In some embodiments, the target-specific luminescent detection agent comprises a target-specific interaction moiety and a luminescent reporter. In such embodiments, interaction of the target-specific interaction moiety with the target analyte (e.g., binding) alters the luminescent signal produced by the luminescent reporter (e.g., increase or decrease). In some embodiments, the degree of alteration of the luminescent signal correlates to the amount of target analyte present and/or the strength of the interaction between the target analyte and the target-specific interaction moiety.

In some embodiments, assay particles provided herein comprise a secondary detection agent. The secondary detection agent is capable of (1) interacting with (e.g., binding to) either (A) the target analyte or (B) the target-specific interaction moiety; and (2) producing a luminescent signal (e.g., alone or in combination with the target-specific luminescent detection agent). In embodiments in which the secondary detection agent is capable of interacting with (e.g., binding to) the target analyte, interaction of the secondary detection agent and the target-specific luminescent detection agent with the target analyte brings the luminescent reporter of the target-specific luminescent detection agent and secondary reporter of the secondary detection agent into proximity. Increase of a combined signal produced by the luminescent reporter and secondary reporter (e.g., resonance energy transfer, bioluminescent structural complementation, etc.) correlates to the presence/concentration of the target analyte. In other embodiments, in which the secondary detection agent is capable of interacting with (e.g., binding to) the target-specific interaction moiety of the target-specific luminescent detection agent, interaction therebetween brings the luminescent reporter of the target-specific luminescent detection agent and secondary reporter of the secondary detection agent into proximity. The target analyte competes with the secondary detection agent for binding to the target-specific interaction moiety, and therefore the increased presence/concentration of the target analyte correlates with a decrease in combined signal produced by the luminescent reporter and secondary reporter (e.g., resonance energy transfer, bioluminescent structural complementation, etc.).

In some embodiments, the target-specific luminescent detection agent comprises a target-specific interaction moiety. A target-specific interaction moiety is any molecular entity capable of interacting with a target analyte in a manner in which a luminescent signal produced by the assay particle (e.g., by the luminescent reporter, by the luminescent reporter and a secondary luminescent reporter) is altered. The type of molecular entities that may be provided as target-specific interaction moieties may vary depending upon the identity of the target and the nature of the intended assay.

In certain embodiments, a target-specific interaction moiety and/or secondary target binding moiety is a target-specific binding moiety. A target-specific binding moiety and/or secondary target binding moiety is a molecular entity capable of binding (e.g., covalently or non-covalently (e.g., stably, transiently)) to a target analyte. Any binding interactions that result in the association of the target analyte with the target-specific interaction moiety (e.g., target-specific binding moiety) are within the scope herein. Exemplary (non-limiting) target-specific binding moieties and/or secondary target binding moieties include, but are not limited to, antibodies, polyclonal antibodies, monoclonal antibodies, recombinant antibodies, antibody fragments, antibody-binding proteins or binding domains thereof (e.g., protein A, Ig binding domain of protein A, protein G, Ig binding domain of protein G, protein A/G, Ig binding domain of protein A/G, protein L, Ig binding domain of protein L, protein M, Ig binding domain of protein M), oligonucleotide probes, peptide nucleic acids, a DARPins, aptamers, affimers, small molecules, protein domains, purified proteins, antigens for antibodies or antibody fragments, protein ligands, etc.

In some embodiments, a target-specific interaction moiety and/or secondary target binding moiety is specific for a single target analyte and discriminates (e.g., does not bind to, does not react with, is not catalyzed by) related analytes from the same or similar class of molecules. For example, a target-specific interaction moiety or secondary target binding moiety may be an antibody or antibody fragment capable of discriminating a target antigen from other similar molecules. In other embodiments, a target-specific interaction moiety or secondary target binding moiety is specific for a class of targets. For example, a target-specific interaction moiety or secondary target binding moiety may be an antibody binding protein capable of binding to the constant region of an entire class of antibodies.

Target-specific interaction moieties are not limited herein to molecular entities capable of binding to a target analyte (although such embodiments are clearly within the scope herein). For example, a target analyte may be an enzyme (e.g., protease), and the target-specific interaction moiety is a substrate for the enzyme (e.g., protease recognition sequence). In such embodiments, interaction of the enzyme with the substrate alters the signal from the luminescent reporter associated with the substrate.

In some embodiments, the target-specific luminescent detection agent comprises a luminescent reporter. Consistent with the definition of luminescence provided herein, a luminescent reporter is a molecular entity capable of emitting light (e.g., upon being induced to do so (e.g., by contacting a substrate, as a result of excitation, etc.) without incandescence (i.e., not as the result of heat)). Examples of luminescent reporters within the scope herein include fluorescent reporters, bioluminescent reporters, and chemiluminescent reporters. In some preferred embodiments, examples of luminescent reporters within the scope herein include, in particular, bioluminescent reporters and chemiluminescent reporters. As is described below, in certain embodiments, the luminescent reporter of a target-specific luminescent detection agent herein functions with a secondary reporter to produce a detectable luminescent signal that is altered upon interaction of the target analyte with the target-specific luminescent detection agent and/or a secondary detection agent. In such embodiments, a luminescent reporter may be a component of a luminescent system (e.g., a RET pair or a component of a complementary complex) rather than a standalone reporter; changes to the system result in changes to the luminescent signal.

In some embodiments, a luminescent reporter (of a target-specific luminescent detection agent) and/or a secondary reporter is a fluorescent reporter. In other embodiments, a component of the spectral signature of an assay particle herein is fluorescent. A fluorescent reporter may be a fluorophore (e.g., small molecule fluorophore or fluorescent protein) capable of producing a detectable signal. In some embodiments, the fluorophore serves as an energy transfer acceptor. In other embodiments the fluorophore is a primary reporter.

In some embodiments, the fluorophore is a small molecule (e.g., molecular weight less than 3,000 daltons, <2,500 daltons, <2,000 daltons, <1,500 daltons, <1,000 daltons, <900 daltons, <800 daltons, <700 daltons, <600 daltons).

Suitable fluorophores for use as fluorescent moieties herein include, but are not limited to: stilbazolium derivatives (Marquesa et al. Mechanism-Based Strategy for Optimizing HALOTAG Protein Labeling. ChemRxiv. Cambridge: Cambridge Open Engage; 2021; incorporated by reference in its entirety), xanthene derivatives (e.g., fluoresceins, rhodamines, rhodols, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), CF dye (Biotium), BODIPY (Invitrogen), ALEXA FLUOR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes) (Columbia Biosciences), etc.

In some embodiments, the fluorophore is a rhodol or rhodamine dye (Beija et al. Chem. Soc. Rev., 2009, 38, 2410-2433; incorporated by reference in its entirety) or a variant or derivative thereof. In some embodiments, the rhodol or rhodamine dye is selected from:

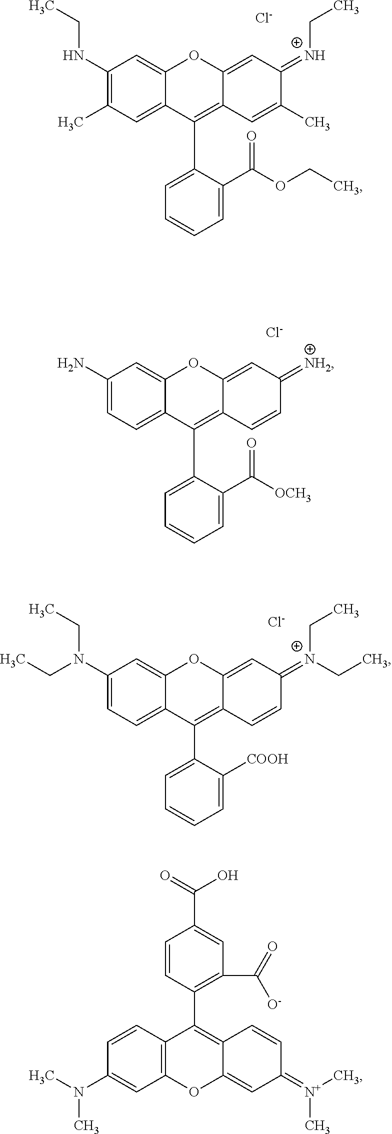

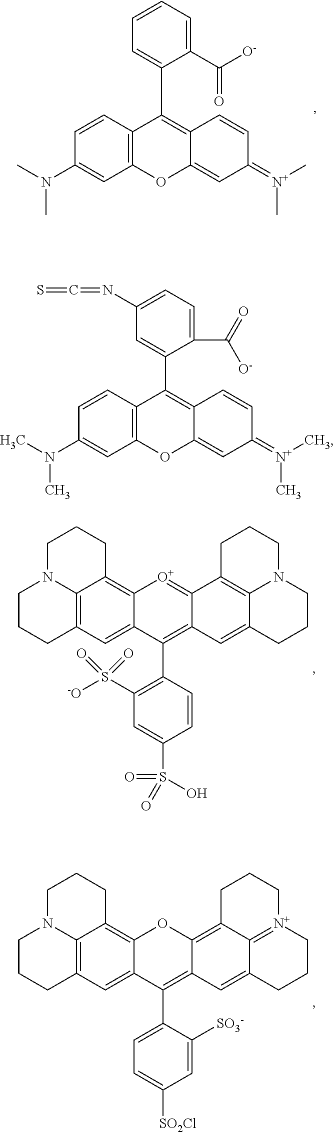

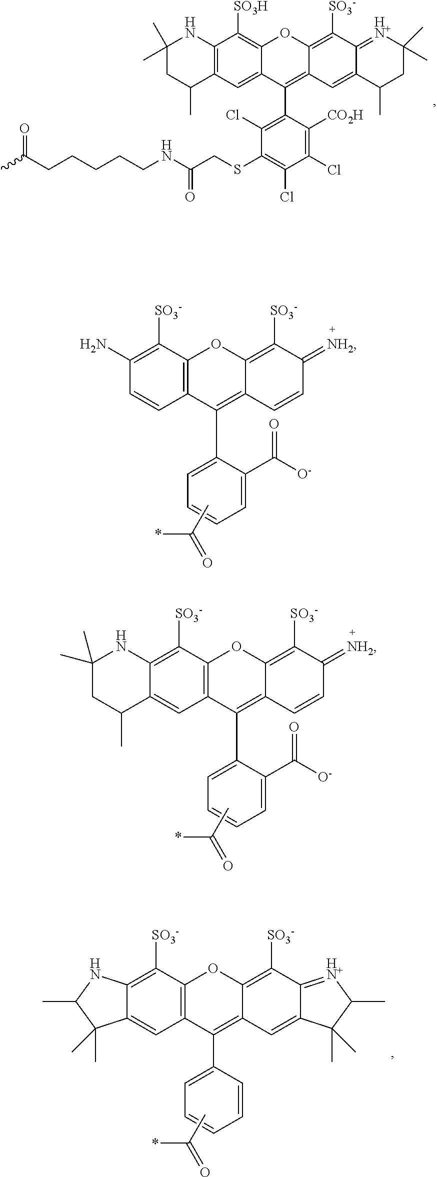

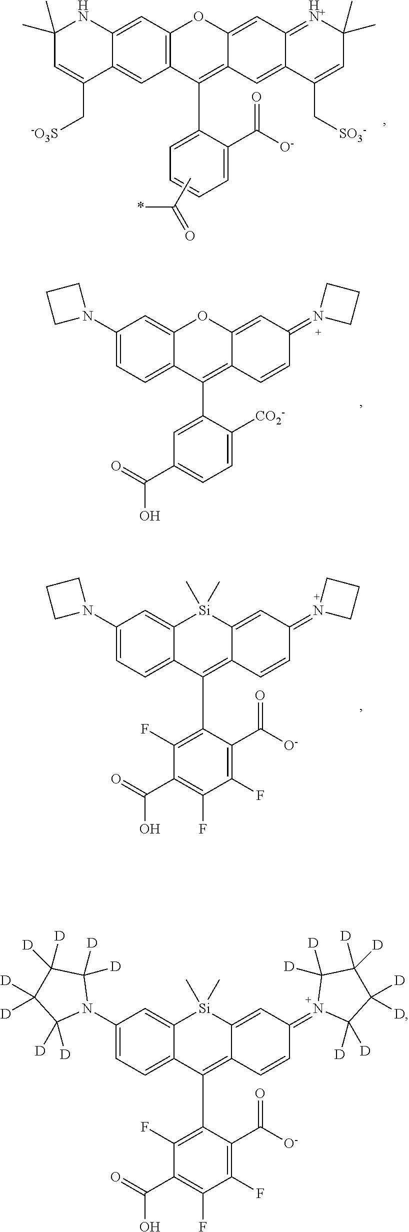

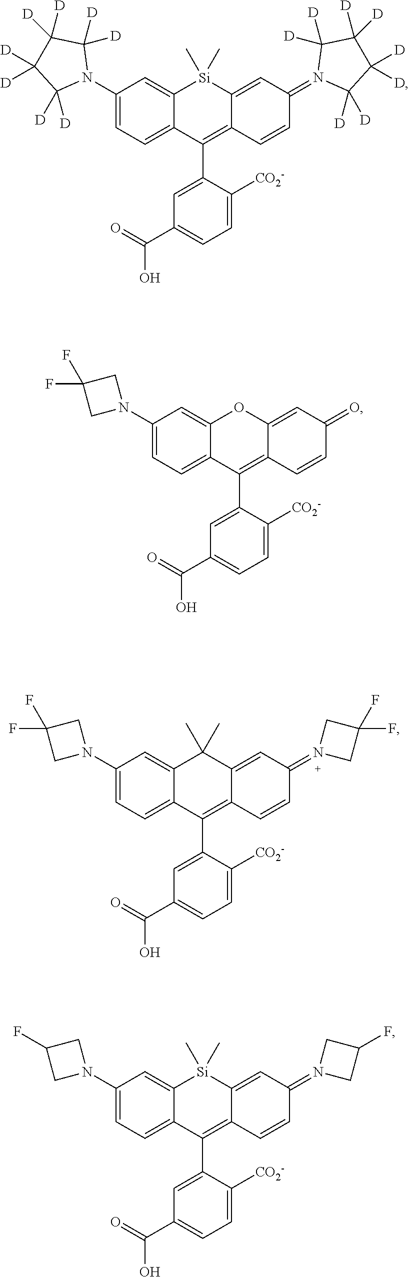

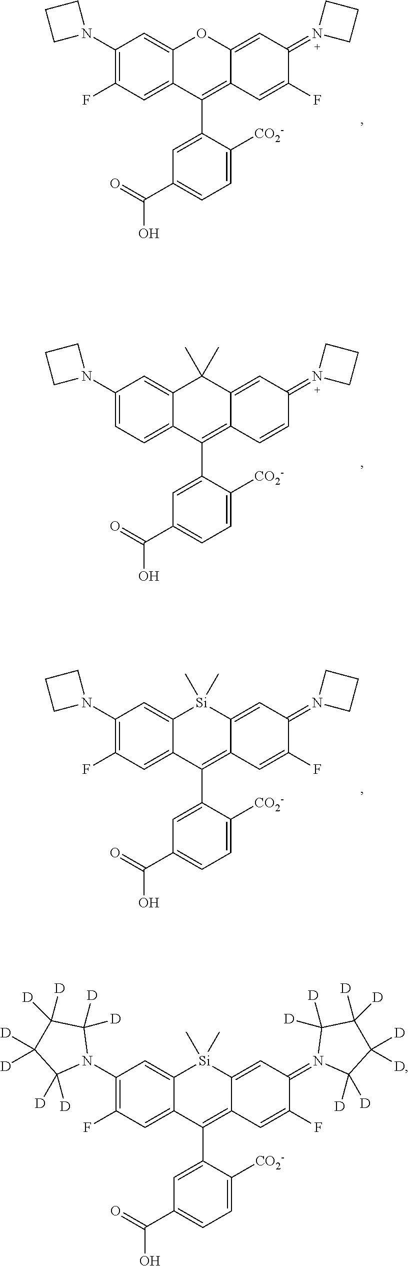

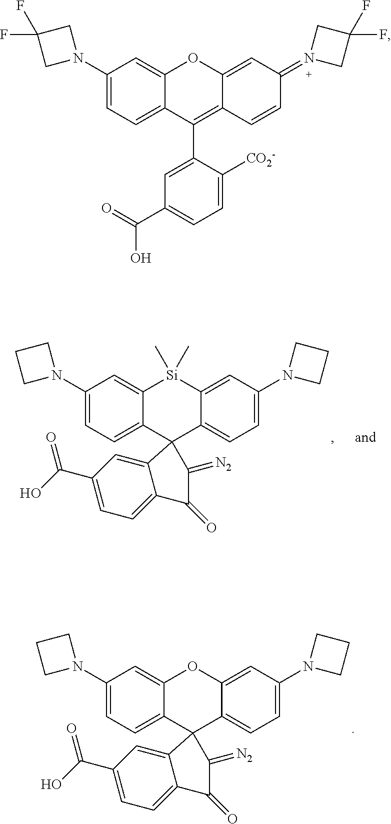

In some embodiments, the fluorophore is a rhodamine.

In some embodiments, a fluorescent moiety is a fluorogenic functional group. A fluorogenic functional group is one that produces and enhanced fluorescent signal upon binding of the substrate to a target (e.g., binding of a haloalkane to a modified dehalogenase). By producing significantly increased fluorescence (e.g., 10×, 31×, 50×, 100×, 310×, 500×, 100×, or more) upon target engagement, the problem of background signal is alleviated. Exemplary fluorogenic dyes for use in embodiments herein include the JANELIA FLUOR family of fluorophores, such as:

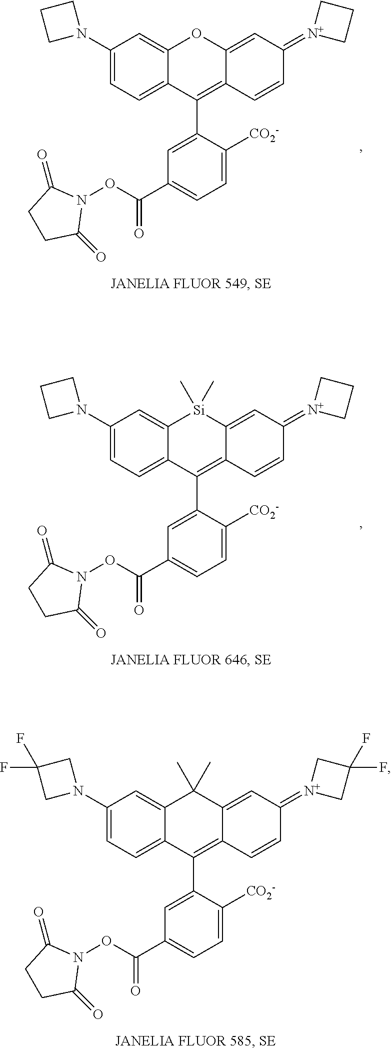

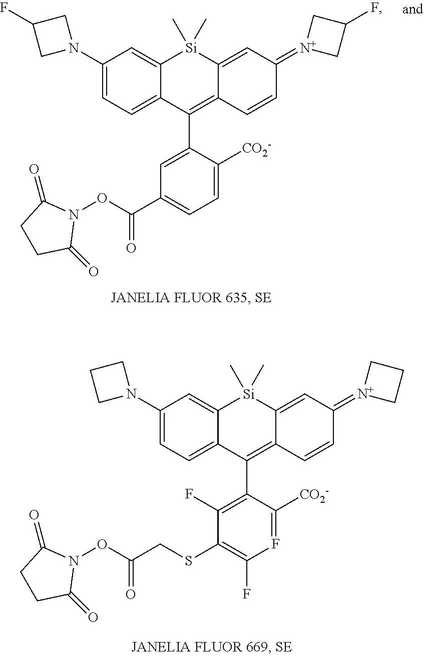

(see, e.g., U.S. Pat. Nos. 9,933,417; 10,018,624; 10,161,932; and 10,495,632; each of which is incorporated by reference in their entireties). In some embodiments, exemplary conjugates of JANELIA FLUOR 549 and JANELIA FLUOR 646 with haloalkane substrates for modified dehalogenase (e.g., HALOTAG) are commercially available (Promega Corp.). The use and design of fluorogenic functional groups, dyes, probes, and substrates is described in, for example Grimm et al. Nat Methods. 3117 October; 14(10):987-994.; Wang et al. Nat Chem. 3120 February; 12(2):165-172; incorporated by reference in their entireties.

In some embodiments, the fluorescent protein is selected from yellow fluorescent protein (YFP), green fluorescent protein (GFP), cyan fluorescent protein (CFP), red fluorescent protein (RFP), umbelliferone, fluorescein, fluorescein isothiocyanate, rhodamine, dichlorotriazinylamine fluorescein, cyanines, dansyl chloride, phycocyanin, and phycoerythrin.

In some embodiments, a fluorescent moiety is a quantum dot. Quantum dots are nanoscale semiconductor particles with unique electronic and optical properties, including size-dependent emission wavelengths. Suitable quantum dots for fluorescent moieties herein include cadmium Selenide (CdSe) quantum dots, lead sulfide (PbS) quantum dots, indium phosphide (InP) quantum dots, silicon (Si) quantum dots, graphene quantum dots (GQDs), zinc selenide (ZnSe) quantum dots, and copper indium sulfide (CuInS₂) quantum dots.

In some embodiments, a luminescent reporter (of a target-specific luminescent detection agent) and/or a secondary reporter is a bioluminescent reporter. In some embodiments, a bioluminescent reporter is a bioluminescent protein. In some embodiments, a bioluminescent protein is a luciferase enzyme. Suitable luciferase enzymes include those selected from the group consisting of: Photinus pyralis or North American firefly luciferase; Luciola cruciata or Japanese firefly or Genji-botaru luciferase; Luciola italic or Italian firefly luciferase; Luciola lateralis or Japanese firefly or Heike luciferase; N. nambi luciferase; Luciola mingrelica or East European firefly luciferase; Photuris pennsylvanica or Pennsylvania firefly luciferase; Pyrophorus plagiophthalamus or Click beetle luciferase; Phrixothrix hirtus or Railroad worm luciferase; Renilla reniformis or wild-type Renilla luciferase; Renilla reniformis Rluc8 mutant Renilla luciferase; Renilla reniformis Green Renilla luciferase; Gaussia princeps wild-type Gaussia luciferase; Gaussia princeps Gaussia-Dura luciferase; Cypridina noctiluca or Cypridina luciferase; Cypridina hilgendorfii or Cypridina or Vargula luciferase; Metridia longa or Metridia luciferase; TurboLuc (Auld et al. Biochemistry 2018, 57, 31, 4700-4706: incorporated by reference in its entirety); Nano-lanterns (Suzuki et al. Nature Communications volume 7, Article number: 13718 (2016); incorporated by reference in its entirety); and Oplophorus luciferase (e.g., Oplophorus gracilirostris (OgLuc luciferase), Oplophorus grimaldii, Oplophorus spinicauda, Oplophorus foliaceus, Oplophorus noraezeelandiae, Oplophorus typus, Oplophorus noraezelandiae or Oplophorus spinous). In some embodiments, a luminescent reporter (of a target-specific luminescent detection agent) and/or a secondary reporter is a luciferase derived from the OgLuc luciferase, such as the NANOLUC® luciferase (Promega Corporation; U.S. Pat. Nos. 8,557,970; 8,669,103; incorporated by reference in their entireties) and engineered luciferases related thereto. PCT Appln. No. PCT/US2010/033449, U.S. Pat. No. 8,557,970, PCT Appln. No. PCT/2011/059018, and U.S. Pat. No. 8,669,103 (each of which is incorporated by reference in their entirety and for all purposes) describe compositions and methods comprising bioluminescent polypeptides. Such polypeptides find use in embodiments herein (e.g., as luminescent reporters and secondary reporters) and can be used in conjunction with the compositions, assays, devices, systems, and methods described herein. In some embodiments, compositions, assays, devices, systems, and methods provided herein comprise a luminescent reporter and/or secondary reporter of SEQ ID NO: 1, or having at least 60% (e.g., 60%, 65%, 70%, 75%, 80%, 85%, 90%, 95%, 96%, 97%, 98%, 99%, 100%, or ranges therebetween) sequence identity with SEQ ID NO: 1. In some embodiments, a luminescent reporter or secondary reporter is a circularly permuted version of a natural or modified bioluminescent protein (See, e.g., U.S. Pat. No. 10,774,364; incorporated by reference in its entirety).

In embodiments herein utilizing bioluminescence (e.g., bioluminescent reporter, bioluminescent secondary reporter, bioluminescent complex of reporter and secondary reporter), suitable luminophore substrates are provided that emit light upon interaction with the bioluminescent proteins and/or complexes described herein. Suitable luminophores for the bioluminescent proteins or complexes used in assay particles or methods herein will be understood. For example, firefly luciferin, with the structure:

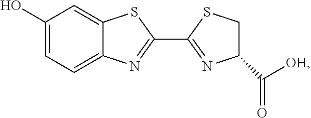 [SMILES: O=C(O)[C@H]1CSC(c2nc3ccc(O)cc3s2)=N1]

is the luciferin found in many Lampyridae species, and is the substrate of beetle luciferases.

Latia luciferin, with the structure:

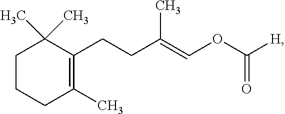

is from the freshwater snail Latia neritoides.

Bacterial luciferin, with the structure:

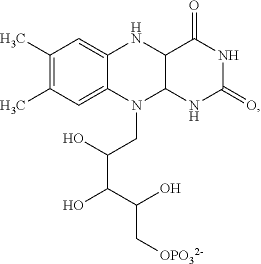

finds use as a substrate for many bacterial luciferases.

Coelenterazine of the structure:

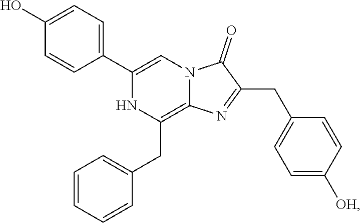 [SMILES: O=c1c(Cc2ccc(O)cc2)nc2c(Cc3ccccc3)[nH]c(-c3ccc(O)cc3)cn1-2]

is found in radiolarians, ctenophores, cnidarians, squid, brittle stars, copepods, chaetognaths, fish, and shrimp, and is the luminophore substrate for the luciferases of those organisms. Variants and derivatives of coelenterazine, such as Furimazine and fluoroFurimazine find use in embodiments herein (e.g., with Oplophorus-derived bioluminescent proteins and complexes).

Other luminophore substrates include those of dinoflagellates:

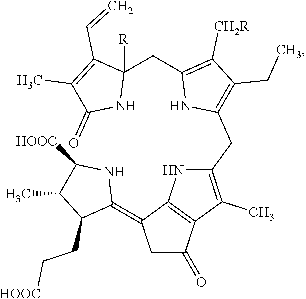

Vargulin (cypridin luciferin):

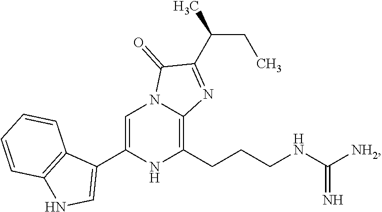 [SMILES: CC[C@H](C)c1nc2c(CCCNC(=N)N)[nH]c(-c3c[nH]c4ccccc34)cn-2c1=O]

and
N. nambi:

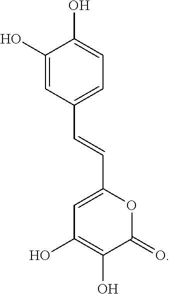 [SMILES: O=c1oc(/C=C/c2ccc(O)c(O)c2)cc(O)c1O]

Pairing of appropriate bioluminescent proteins or complexes with luminophores is understood in the field. In particular embodiments, a bioluminescent protein is provided component of an assay particle or assay herein that utilizes an imidazopyrazine luminophore, such as coelenterazine, Furimazine, or fluoroFurimazine (U.S. application Ser. No. 16/548,214; incorporated by reference in its entirety). In some embodiments, a system or method herein comprises (1) an Oplophorus-derived polypeptide (e.g., NANOLUC) or components of an Oplophorus-derived bioluminescent complex (e.g., NANOBIT, NANOTRIP) and an imidazopyrazine luminophore (e.g., coelenterazine, Furimazine, fluoroFurimazine, etc.). In some embodiments, systems and methods herein comprise an imidazopyrazine luminophore such as native coelenterazine, Furimazine, fluoroFurimazine, hydroFurimazine, coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); and U.S. Pat. No. 8,669,103; the disclosures of which are incorporated by reference herein in their entireties.

In some embodiments, the luminophore emits light upon interaction with the bioluminescent protein or complex. In some embodiments, the luminophore emits light in the visible light spectrum (e.g., about 400 to about 700 nm (e.g., 400 nm, 425 nm, 450 nm, 475 nm, 500 nm, 525 nm, 550 nm, 575 nm, 600 nm, 625 nm, 650 nm, 675 nm, 700 nm, or ranges therebetween). In some embodiments, the luminophore emits light of a wavelength between 400 and 500 nm (e.g., 400 nm, 410 nm, 420 nm, 430 nm, 440 nm, 450 nm, 460 nm, 470 nm, 480 nm, 490 nm, 500 nm, or ranges therebetween).

In some preferred embodiments, a bioluminescent reporter and/or secondary reporter is selected from NANOLUC (e.g., thermostable (TS) and non-thermostable variants), TURBOLUC, Firefly luciferase (FLuc), Ultra-Glo recombinant luciferase, Renilla luciferase (RLuc), Gaussia luciferase (GLuc), Click Beetle luciferase, and Fungal luciferase (Luz). Accordingly, each luciferase-based biosensor is used with its key cognate substrates including, but not limited to: Furimazine (Fz), Fluoro-Fz (FFz), Cephalo-Fz (CFz/CFz-9), hydroFurimazine, coelenterazine (Cz), D-Luciferin, etc.

In some embodiments, luminophore substrates are provided as pro-substrates. Pro-substrates are inactive forms of luminophores that cannot support luminescence directly when combined with the appropriate luciferase, but can be converted into an active substrate through processing (e.g., chemical, enzymatic, light-induced, pH-induced, etc.). In some embodiments, the pro-substrate is converted by the enzyme or chemical reagent to be detected by the assay. In other embodiments, the pro-substrate is converted by the enzyme or chemical reagent intended to trigger analyte detection by converting the pro-substrate into an active substrate. For example, a substrate can be converted into a pro-substrate by inclusion of a blocking group that prevents suitable interaction with a luciferase; upon chemical or enzymatic removal of the blocking group, the pro-substrate is converted into the active substrate, thereby triggering detection of the luminescent signal from the assay particle. Non-enzymatic methods of converting pro-substrates to active luminophore substrates include utilizing pH, heat, chemical activators, or light for homogenous and instant release. Enzymatic conversion of pro-substrates to active luminophore substrates can be performed by various enzymes such as esterases, proteases, monoamine oxidases (MAO), P450, and glutathione S-transferase (GST). Any luminophore substrates described herein or understood in the field can be provided in embodiments herein as pro-substrates.

In some embodiments, a luminescent reporter (of a target-specific luminescent detection agent) and/or a secondary reporter is a chemiluminescent reporter. Examples of chemiluminescent reporters that may find use as luminescent reporters (of a target-specific luminescent detection agent) and/or a secondary reporters herein include horseradish peroxidase, alkaline phosphatase, lactase, glucose oxidase, catalase, acid phosphatase, nitrate reductase, glutathione peroxidase, choline oxidase, or singlet oxygen molecule generators and acceptors.

In some embodiments, a luminescent reporter of a target-specific luminescent detection agent is the sole detectable reporter to which interaction of the target analyte with the target-specific interaction moiety is correlated. In other embodiments, a detectable signal (or change thereof) that can be correlated to interaction of the target analyte with the target-specific interaction moiety is achieved by the interaction of a luminescent reporter of a target-specific luminescent detection agent and a secondary reporter of a secondary detection agent. Exemplary mechanisms of interaction between the target-specific luminescent detection agent and a secondary reporter include structural complementation and resonance energy transfer (e.g., Förster resonance energy transfer (FRET), bioluminescence resonance emery transfer (BRET), etc. In some embodiments, a luminescent reporter (of a target-specific luminescent detection agent) and a secondary reporter are components of a luminescent system (e.g., structural complementation system, energy transfer system (e.g., BRET, FRET, etc.), etc.

Bioluminescent structural complementation is the formation of a bioluminescent complex from two or more peptide/polypeptide components. The bioluminescent signal produced by the complex in the presence of a luminophore substrate is significantly enhanced (e.g., 2-fold, 5-fold, 10-fold, 20-fold, 50-fold, 100-fold, 200-fold, 500-fold, 1000-fold, or more) compared to any of the individual components and the luminophore substrate.

The commercially-available NANOBIT complementation system (Promega) is an exemplary bioluminescent structural complementation system for use in certain embodiments herein. The native Oplophorus gracilirostris luciferase (OgLuc) and commercially-available NANOLUC luciferase (Promega Corporation) each comprise polypeptides of 10 β (beta) strands (β1, β2, β3, β4, β5, β6, β7, β8, β9, 10). U.S. Pat. No. 9,797,889 (incorporated by reference in its entirety) describes development and use of a complementation system comprising a β1-9-like polypeptide and a β10-like peptide (certain OgLuc/NANOLUC-based polypeptide and peptide sequences in polypeptide and peptide sequences in U.S. Pat. No. 9,797,889 differ from the corresponding sequences in NANOLUC and wild-type native OgLuc). Similarly, U.S. application Ser. No. 16/439,565 (incorporated by reference in its entirety) describes the development and use of a complementation systems comprising two or more OgLuc/NANOLUC peptides and/or polypeptides (certain OgLuc/NANOLUC-based polypeptide and peptide sequences in U.S. patent Ser. No. 16/439,565 differ from the corresponding sequences in NANOLUC and wild-type native OgLuc).

In some embodiments, a luminescent reporter herein comprises a first component (e.g., peptide or polypeptide) of a bioluminescent complex that is capable of associating through structural complementation with a secondary reporter comprising a second component (e.g., polypeptide or peptide) of the bioluminescent complex to form a complex that is capable of activating a luminophore to emit light. In some embodiments, a luminophore emits significantly more light in the presence of the bioluminescent complex than in the presence of any one of the components alone. In some embodiments, a bioluminescent complex is formed from fragments (e.g., peptide(s) and/or polypeptide(s)) of a luciferase enzyme (e.g., natural or synthetic luciferase). In some embodiments, a bioluminescent complex is a circularly permuted version of a natural or modified bioluminescent component (e.g., formed from two fragments of a circularly permuted luciferase); See, e.g., U.S. Pat. No. 10,774,364; incorporated by reference in its entirety. PCT Appln. Nos. PCT/US14/26354, PCT/US19/036844, and PCT/US20/62499; U.S. Pat. No. 9,797,889; U.S. patent application Ser. No. 16/439,565; and U.S. Pub. No. 2021/0262941 (each of which is incorporated by reference in their entirety and for all purposes) describe compositions and methods for the assembly of bioluminescent complexes; such complexes, and the peptide and polypeptide components thereof, find use in embodiments herein and can be used in conjunction with the assays and methods described herein.

In some embodiments, a luminescent reporter and secondary reporter comprise peptide and polypeptide components for the assembly of a bioluminescent complex capable of generating luminescence in the presence of an appropriate substrate (e.g., a coelenterazine or a coelenterazine analog (e.g., Furimazine, fluoroFurimazine, etc.). In some embodiments, complementary polypeptide(s) and peptide(s) collectively span the length (or >75% of the length, >80% of the length, >85% of the length, >90% of the length, >95% of the length, or more) of a luciferase base sequence (or collectively comprise at least 40% sequence identity to a luciferase base sequence (e.g., >40%, >45%, >50%, >55%, >60%, >65%, >70%, >75%>80%, >85%, >90%, >95%, or more). In some embodiments, “complementary” polypeptide(s) and peptide(s) are separate molecules that each correspond to a portion of a luciferase base sequence. Through structural complementarity, the luminescent reporter and the secondary reporter assemble to form a bioluminescent complex. Suitable luciferase base sequences may include SEQ ID NOS: 1 or 2, or the sequences of any of the full-length luciferases listed above. In some embodiments, the bioluminescent complex comprises the NANOBIT or NANOTRIP systems (Promega; Madison, WI). In some embodiments, the peptide and/or polypeptide components of a bioluminescent complex collectively comprise at least 60% sequence identity (e.g., >60%, >65%, >70%, >75%, >80%, >85%, >90%, >95%, >99%) with SEQ ID NO: 1 and/or SEQ ID NO: 2. In some embodiments, the peptide and/or polypeptide components of the bioluminescent complex comprise HiBiT (SEQ ID NO: 3), SmBiT (SEQ ID NO: 4), LgBiT (SEQ ID NO: 5), LgTRIP (SEQ ID NO: 6), and/or SmTRIP9 (SEQ ID NO: 7). In some embodiments, the peptide and/or polypeptide components of the bioluminescent complex comprise at least 60% sequence identity (e.g., >60%, >65%, >70%, >75%, >80%, >85%, >90%, >95%, >99%) with HiBiT (SEQ ID NO: 3), SmBiT (SEQ ID NO: 4), LgBiT (SEQ ID NO: 5), LgTRIP (SEQ ID NO: 6), and/or SmTRIP9 (SEQ ID NO: 7).

Embodiments herein utilizing bioluminescent structural complementation as a luminescent reporter system are not limited to NANOBIT, NANOTRIP, or other NANOLUC related systems. Components of any split luciferase may find use as a luminescent reporter and secondary reporter in assay particles herein. Other split protein reporters and/or complementation systems that are understood in the field and may find use in embodiments herein and/or inform complementation systems that may find use in embodiments herein include split β-lactamase, split β-galactosidase, split dihydrofolate reductase, split green fluorescent protein (Cabantous et al., Sci Rep 3, 2854 (2013); incorporated by reference in its entirety), split ubiquitin, and split TEV protease (Morrell et al., FEBS Lett 583, 1684-91 (2009); incorporated by reference in its entirety).

Resonance energy transfer is a mechanism by which energy is transferred from a donor molecule to an acceptor molecule through nonradiative dipole-dipole coupling. The energy is then emitted from the acceptor molecule, typically in the form of luminescence (e.g., fluorescence). The efficiency of this energy transfer is inversely proportional to the sixth power of the distance between donor and acceptor, making it extremely sensitive to small changes in distance. Förster resonance energy transfer (FRET) typically refers to the transfer of energy from one fluorescent molecule (e.g., fluorophore, fluorescent protein, etc.) to a second fluorescent molecule. The first fluorescent molecule is excited by exposure to light within the excitation spectrum of the first fluorescent molecule, energy is transferred via FRET to a second fluorescent molecule with an excitation spectrum that overlaps with the emission spectrum of the first fluorescent molecule, and then the second fluorescent molecule emits the energy as light within the emission spectrum of the second fluorescent molecule. Bioluminescent resonance energy transfer (BRET) typically refers to the transfer of energy from a bioluminescent protein/complex and luminophore substrate molecule to an acceptor fluorescent molecule (e.g., fluorophore, fluorescent protein, etc.). The luminophore substrate is excited by enzymatic action of the bioluminescent protein/complex, energy is transferred via BRET to the acceptor fluorescent molecule with an excitation spectrum that overlaps with the emission spectrum of the bioluminescent protein/complex and luminophore, and then the fluorescent molecule emits the energy as light within the emission spectrum of the fluorescent molecule.

In some embodiments, a luminescent reporter of a target-specific luminescent detection agent is a FRET or BRET donor (or a component of a bioluminescent donor complex), and a secondary reporter of a secondary detection agent is an acceptor. In other embodiments, a secondary reporter of a secondary detection agent is a FRET or BRET donor (or a component of a bioluminescent donor complex) and a luminescent reporter of a target-specific luminescent detection agent is an acceptor. Any bioluminescent proteins (e.g., luciferases (e.g., NANOLUC, Firefly, etc.)) and/or bioluminescent complexes (e.g., bioluminescent structural complementation systems (e.g., NANOBIT, NANOTRIP, etc.)) described herein find use as donors in BRET reporter systems (e.g., as luminescent reporter or secondary reporter) of assay particles herein. Any fluorophores and/or fluorescent proteins described herein find use as donors in FRET reporter systems (e.g., as luminescent reporter or secondary reporter) and/or acceptors in BRET or FRET systems of assay particles herein. Criteria for selecting appropriate donor/acceptor pairs for excitation/emission in BRET or FRET systems is understood in the field.

In some embodiments, provided herein are assay particles comprising a secondary detection agent. A secondary detection agent comprises (i) a secondary reporter (e.g., a fluorophore), and (ii) typically (A) a secondary target binding moiety, or (B) a target mimetic. Whether functioning through structural complementation or resonance energy transfer, the secondary reporter is brought into proximity of the luminescent reporter of the target-specific interaction element to produce a combined signal. While the target-specific interaction moiety places the luminescent reporter in proximity of the target analyte, a secondary detection agent may function through multiple different mechanisms to place the secondary reporter in proximity of the luminescent reporter of the target-specific interaction agent.

In some embodiments, a secondary detection agent comprises (i) a secondary reporter (e.g., a fluorophore, complementary component of a luminescent reporter, etc.) and (ii) a secondary target binding moiety. In such embodiments, upon interaction of the target-specific interaction moiety of the target-specific interaction agent with the target analyte, the luminescent reporter is placed in proximity of the target analyte. Then, upon binding of the secondary target binding moiety to the target analyte, the secondary reporter is placed in proximity of the luminescent reporter, and resonance energy transfer or structural complementation between the luminescent reporter and the secondary reporter can occur.

In some embodiments, a secondary detection agent comprises (i) a secondary reporter (e.g., a fluorophore, complementary component of a luminescent reporter, etc.) and (ii) a target mimetic. In certain embodiments comprising such a system, the target-specific interaction moiety of the target-specific interaction agent is allowed to bind to the target mimetic of the secondary detection agent; upon such binding, the luminescent reporter and secondary reporter are placed in proximity for resonance energy transfer or for structural complementation between the luminescent reporter and the secondary reporter to occur. Then, upon exposure of the assay particle to the target analyte, the target analyte completes with the target mimetic of the secondary detection agent, thereby decreasing the resonance energy transfer or structural complementation between the luminescent reporter and the secondary reporter.

In typical embodiments, a population of assay particles (e.g., equivalent assay particles) is provided. A population may comprise 10-10,000 individual assay particles (e.g., 10, 20, 50, 100, 200, 500, 1,000, 2,000, 5,000, 10,000, or ranges or values therebetween). In some embodiments, all of the assay particles within a population of assay particles comprise the same components (e.g., same target-specific luminescent detection agent, same secondary detection agent, etc.). In some embodiments, a population of assay particles (e.g., equivalent assay particles) comprises two or more (e.g., 2, 3, 4, 5, 6, 7, 8, 9, 10, 20, 30, 40, 50, or more or ranges therebetween) subsets of distinct assay particles. Typically, all of the assay particles in the population, regardless of subset, comprise substantially the same physical/structural makeup (e.g., matrix material, percent interstitial void volume, crosslinking concentration, etc.). All of the assay particles in the population, regardless of subset, may also contain certain substantially identical assay reagents, such as the luminescent reporter portion of the target-specific luminescent detection agent, the secondary reporter portion of the secondary detection agent, the entire secondary detection agent, linkers, etc. However, the assay particles of different subsets of a population of equivalent assay particles typically differ in at least the identity of the target-specific moiety portion of the target-specific luminescent detection agent and the assay-particle-identifying physical characteristic. The assay particles of different subsets of a population may also optionally differ in the identity of the secondary binding moiety portion of a secondary detection agent. In some embodiments, a unique target-specific moiety (and/or secondary binding moiety) allows the assay particles of a subset to bind to a different target analyte than the assay particles of a different subset. In other embodiments, the unique target-specific moiety (and/or secondary binding moiety) allows the assay particles of a subset to bind to the same target analyte as the assay particles of a different subset, but with different affinity, specificity, at a different biding location (e.g., epitope), etc. Because the assay particles of different subsets with the population have the same luminescent reporters and/or secondary reporters, the signal produced as a readout for an assay performed with the assay particles is not distinguishable between the different subsets; however, because each subset has a unique assay-particle-identifying physical characteristic (e.g., spectral signature) that is distinguishable from the -particle-identifying physical characteristics of other subsets of the population, the readout of each particle can be correlated to the subset to which it belongs by identifying the -particle-identifying physical characteristic of the assay particle(s). In some embodiments, inclusion of multiple subsets of assay particles in a population allows for multiplex assays to be performed in a population of assay particles in a single sample or reaction mixture.

The assay particles and systems herein can be flexibly fitted for different assays and detection modalities due to the modularity of being able to tether different biosensors (e.g., different target-specific interaction moieties, different secondary detection agent, etc.) to the assay particles. In some embodiments, the assay particles herein utilize non-overlapping methods of chemical conjugation that allow for the more multi-component biosensors (e.g., utilizing structural complementation (e.g., based on NanoTrip, NanoBiT, etc.), BRET (e.g., based on NanoBRET), etc.) to be stoichiometrically introduced and optimized for specific applications. In some embodiments, the desired components for a particular assay (e.g., target-specific interaction moieties, secondary detection agents, etc.) are tethered onto assay particles utilizing varying aspects of conjugation chemistry. Adjusting the length and composition of linkers used in conjugation can influence the flexibility, stability, and spatial orientation of the assay components. Linkers may include chemical linkers (e.g., alkyl chains, heteroalkyl chains, polyethylene glycols, etc.) amino acid linkers etc. Optimizing linkers can enhance the efficiency of signal transduction and accessibility to target analytes. Any components herein, particularly those as being described as conjugated, linked, or tethered to another component and/or the matrix material may be connected to a linker. A wide variety of linkers can be used in tethering components described herein to each other and/or to the assay particles (e.g., to the matrix materials).

In some embodiments, chemical linkers are provided. In some embodiments, chemical linkers find particular use in tethering small molecule components (e.g., fluorophores, ligands, substrates, target mimetics, HALOTAG ligand, biotin, etc.) to each other and/or to the matrix material. Exemplary chemical linkers may comprise functional groups selected alkylene, arylene, —O—, —NH—, carbamate, and —C(O)— groups. For example, linkers may include various combinations of such groups to provide linkers having ester (—C(O)O—), amide (—C(O)NH—), carbamate (—NHC(O)O—), urea (—NHC(O)NH—), phenylene (e.g., 1,4-phenylene), straight or branched chain alkylene, and/or oligo- and poly-ethylene glycol (—(CH₂CH₂O)ₓ—) linkages, and the like. In some embodiments, the linker may include 2 or more atoms (e.g., 2-200 atoms, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 atoms, or any range therebetween (e.g., 2-20, 5-10, 15-35, 25-100, etc.)).

In some embodiments, linkers are stable covalent bonds between conjugated molecules, and may include alkyl, aryl, heteroaryl groups, or other functional groups. In some embodiments, linkers include groups to impart solubility and/or reduce aggregation. For instance, polyethylene glycol (PEG) linkers, PEG derivatives, and polyglutamine can enhance solubility and biocompatibility. Hydrophobic linkers, such as alkyl chains or aromatic groups, may be utilized to enhance stability or promote specific interactions with insoluble components.

In some embodiments, linkers are formed between reactive functional groups on the two components being linked, such as thiols (—SH), amines (—NH₂), carboxyls (—COOH), or isocyanates. Examples include N-hydroxysuccinimide (NHS) esters, maleimides, disulfide bonds, thioether linkers, and biaryl linkers.

Peptide linkers find use in tethering of components herein, particularly in the joining of two peptide or polypeptide components. The use of specific peptide sequences, often consisting of flexible amino acid residues, provides an effective method for linking biomolecules while maintaining functionality. Typical amino acid linkers include flexible sequences composed of glycine (Gly), serine (Ser), glutamine (Gln), asparagine (Asn), and proline (Pro) residues. For example, (Gly) n (where n is an integer, typically between 1 and 10) can be used to provide flexibility and reduce steric hindrance. Other peptide linkers may include sequences like GGGGS (a commonly used flexible linker sequence) or (Ser) 4, which have been optimized for their non-interfering nature and ability to maintain protein structure.

In certain embodiments, a component is functionalized with a moiety to allow for conjugation to the matrix material and/or another component (e.g., with a complimentary functional group). The conjugation may be covalent or non-covalent, depending on the application herein. Exemplary chemistries for covalent conjugation include N-hydroxysuccinimide (NHS) esters and various click chemistries. NHS esters react with primary amines to form stable amide bonds, thereby providing a stable and permanent attachment, suitable for long-term assays. Strain-promoted azide-alkyne cycloaddition (SPAAC) is a bio-orthogonal conjugation strategy that does not require a copper catalyst, thereby avoiding toxicity. Copper-catalyzed azide-alkyne cycloaddition (CuAAC) is a highly efficient conjugation strategy using a copper catalyst to form triazoles that provides robust and specific conjugation. Inverse electron-demand diels-alder (IEDDA) is a fast and biocompatible conjugation strategy between a dienophile and a 1,2,4,5-tetrazine that proceeds rapidly under physiological conditions.

In some embodiments, a linker is cleavable by any suitable chemistry, for example by light, chemical reaction, heat, pH, exposure to an enzyme, radical oxygen species, biorthogonal chemistries, etc. Cleavable linkers may find use for applications in which separation of components within the assay particle is required. The components are held separate by being tethered to the matrix material; upon cleaving of one or both linkers, the components are released into the interstitial void volume and allowed to interact with each other and/or other components. Cleavable linkers are designed to undergo cleavage under specific conditions, allowing for the reversible attachment of components (e.g., to each other or the matrix material). Cleavage can be triggered by various stimuli, including light, chemical reactions, pH changes, heat, phase change, mechanical force, electric stimuli, enzymatic activity, etc.

Light-sensitive linkers are useful in applications requiring spatial and temporal control over the cleavage event. These linkers can be cleaved upon exposure to light of specific wavelengths. Nitrobenzyl-based linkers are cleaved upon exposure to UV or visible light. A typical example is the nitrobenzyl ester or nitrobenzyl ether linkage, which undergoes homolytic bond cleavage upon light exposure, releasing the conjugated biomolecule. Caged linkers, such as those based on ortho-nitrobenzyl groups, are also cleaved upon exposure to specific wavelengths of light (usually UV), these linkers release the attached molecule or fragment. Benzoin ether linkers are cleaved under UV light.

Chemical reaction-cleavable linkers undergo cleavage via specific chemical reactions, which may be triggered by a variety of conditions. These reactions are useful in applications where precise control over the release of conjugated biomolecules is required, such as in drug delivery systems or targeted therapies. Disulfide bonds, formed between two cysteine residues or cysteine-like groups, are highly cleavable under reducing conditions. The reduction of the disulfide bond by agents like dithiothreitol (DTT) or tris(2-carboxyethyl)phosphine (TCEP) leads to cleavage of the linker. This type of linker is commonly used in protein conjugation (e.g., between peptide/polypeptide groups and/or linking a peptide/polypeptide to a non-peptidic moiety). Hydrazone and hydrazide linkers can be cleaved under mildly acidic conditions, typically at pH values between 4 and 6. Acylhydrazone linkers can be cleaved in the presence of nucleophiles like amines or in the presence of reducing agents.

Heat-sensitive linkers undergo cleavage when exposed to elevated temperatures. Thermoresponsive polymers, like poly(N-isopropylacrylamide) (PNIPAM), can undergo a transition at specific temperatures, leading to either reversible or irreversible cleavage. Thermolabile esters, such as benzyl esters, are cleavable upon exposure to heat, allowing for controlled release in heat-sensitive applications.

pH-sensitive linkers are cleaved under acidic or basic conditions. Hydrazone and acetals can be used as acid-sensitive cleavable linkers. For example, the hydrazone bond between an aldehyde or ketone group and a hydrazine is stable at neutral pH but undergoes cleavage in acidic environments (typically pH 4-6). Imidazole-containing linkers undergo protonation at low pH, leading to cleavage of the bond. Acetal or ketal groups, such as 1,2-dioxane derivatives, can be cleaved when exposed to acidic pH.

Enzyme-sensitive linkers exploit the specificity of enzymes to cleave the linker and release conjugated molecules in a controlled manner. Peptide sequences that are cleavable by specific enzymes are commonly used as linkers. For instance, the use of linkers cleavable by matrix metalloproteinases (MMPs), cathepsins, or plasmin enables selective cleavage in the presence of these enzymes. A typical example is the use of Val-Cit (valine-citrulline) sequences, which are cleaved by cathepsin B or other proteases. Other protease-sensitive linkers, such as Gly-Arg, Pro-Gly, or Leu-Leu, are cleaved by enzymes like furin, trypsin, or proteinase K. Peptoid linkers can be designed to be cleavable by specific enzymes, providing high specificity and control over cleavage.

Self-labeling proteins are engineered proteins that can covalently bind to specific small molecules, and find use in various labeling and conjugation application herein. The protein component of a self-labeling system may be referred to herein as a “capture agent” and the small molecule as the “capture ligand.” In some embodiments, the capture agent (e.g., modified dehalogenase (e.g., HALOTAG)) is linked to, or is provided as a fusion with, another component of the systems herein (e.g., reporter (e.g., NANOLUC, LgBiT, SmBiT, HiBiT, etc.), target-specific interaction moiety, etc.). In some embodiments, the capture ligand (e.g., haloalkyl group) is linked to another component of the systems herein (e.g., target mimetic, fluorophore, ligand, etc.). In such embodiments, when the capture agent binds to the capture ligand, the associated components are thereby linked together.

In some embodiments, the self-labeling protein (e.g., capture agent) is a SNAP-tag protein. SNAP-tag is a modified version of the 06-alkylguanine-DNA alkyltransferase (AGT), a naturally occurring DNA repair protein that can transfer alkyl groups from damaged DNA to itself. The SNAP-tag technology was developed to exploit the enzyme's ability to covalently attach small molecules to its active site. The SNAP-tag protein contains a mutant version of AGT that has been modified to covalently bind to a synthetic substrate, often referred to as 06-benzylguanine derivatives. The most common substrate is 06-benzylguanine (BG), which contains a benzyl group that forms a stable covalent bond with the SNAP-tag at its active site. CLIP-Tag is a variant of the Snap-Tag that reacts with benzylcytosine derivatives. In some embodiments, SNAP-tag is used to label proteins fused thereto with a small molecule (e.g., fluorescent dye, ligand, etc.) or other moiety (e.g., matrix material) that is conjugated to the 06-benzylguanine substrate.

In some embodiments, the self-labeling protein (e.g., capture agent) is a dehalogenase that has been modified to covalently bind to its haloalkyl substrate (e.g., HALOTAG). In some embodiments, the modified dehalogenase is the commercially-available HALOTAG protein (SEQ ID NO: 8), or a variant thereof (e.g., >70% sequence identity). HALOTAG is a 297-residue self-labeling polypeptide (33 kDa) derived from a bacterial hydrolase (dehalogenase) enzyme, which has modified to covalently bind to its ligand, a haloalkane moiety. The HALOTAG ligand can be linked to the matrix material or functional groups (e.g., fluorophores, ligands, target mimetics, etc.), and the HALOTAG polypeptide can be fused to various proteins, allowing covalent attachment of the protein to the matrix material or functional group. The HALOTAG polypeptide is a hydrolase with a genetically modified active site, which specifically binds to the haloalkane ligand chloroalkane linker with an enhanced and increased rate of ligand binding (Pries et al. The Journal of Biological Chemistry. 270(18):10405-11; incorporated by reference in its entirety). The reaction that forms the bond between the protein tag and chloroalkane linker is fast and essentially irreversible under physiological conditions (Waugh D S (June 2005). Trends in Biotechnology. 23(6):316-20; incorporated by reference in its entirety). In the natural hydrolase enzyme, nucleophilic attack of the chloroalkane reactive linker causes displacement of the halogen with an amino acid residue, which results in the formation of a covalent alkyl-enzyme intermediate. This intermediate would then be hydrolyzed by an amino acid residue within the wild-type hydrolase (Chen et al. (February 2005) Current Opinion in Biotechnology. 16(1):35-40; incorporated by reference in its entirety). This would lead to regeneration of the enzyme following the reaction. However, with HALOTAG, the modified haloalkane dehalogenase, the reaction intermediate cannot proceed through the second reaction because it cannot be hydrolyzed due to the mutation in the enzyme. This causes the intermediate to persist as a stable covalent adduct with which there is no associated back reaction (Marks et al. (August 2006) Nature Methods. 3 (8): 591-6; incorporated by reference in its entirety). Various HALOTAG ligands, functional groups, fusions, assays, modifications, uses, etc. are described in U.S. Pat. Nos. 8,748,148; 9,593,316; 10,246,690; 8,742,086; 9,873,866; 10,604,745; U.S. Pat. App. 2009/0253131; U.S. Pat. App. 2010/0273186; 20130337539; U.S. Pat. App. 2012/0258470; U.S. Pat. App. 2012/0252048; U.S. Pat. App. 2011/0201024; U.S. 2014/0322794; each of which is incorporated by reference in their entireties. In some embodiment, a modified dehalogenase for use in embodiments herein has one or more extended surface loop regions that provide a location for internal fusion insertions and modulate binding interaction and activation of environmentally-sensitive chemistries, such as those described in International Patent Application PCT/US2023/021041 (incorporated by reference in its entirety).

The modified dehalogenases herein (e.g., HALOTAG) utilize haloalkane substrates. In some embodiments, the substrate is of formula (I): R-linker-A-X, wherein R is the matrix material, one or more functional groups, or absent, wherein the linker is a multiatom straight or branched chain including C, N, S, or O, or a group that comprises one or more rings, e.g., saturated or unsaturated rings, such as one or more aryl rings, heteroaryl rings, or any combination thereof, wherein A-X is a substrate for a dehalogenase, hydrolase, HALOTAG, or a modified dehalogenase system herein (e.g., wherein A is (CH₂)₄₋₂₀ and X is a halide (e.g., Cl or Br)). Suitable substrates are described, for example, in U.S. Pat. Nos. 11,072,812; 11,028,424; 10,618,907; and 10,101,332; incorporated by reference in their entireties. In some embodiments, R is one or more functional groups (such as a fluorophore, biotin, luminophore, ligan, target mimetic, etc.). Exemplary functional groups include, but are not limited to, an amino acid, protein, e.g., enzyme, antibody or other immunogenic protein, a radionuclide, a nucleic acid molecule, a drug, a lipid, biotin, avidin, streptavidin, an electron opaque molecule, chromophore, a dye, e.g., a xanthene dye, a calcium sensitive dye, e.g., 1-[2-amino-5-(2,7-dichloro-6-hydroxy-3-oxy-9-xanthenyl)-phenoxy]-2-(2′-amino-5′-methylphenoxy)ethane-N,N,N′,N′-tetraacetic acid (Fluo-3), a sodium sensitive dye, e.g., 1,3-benzenedicarboxylic acid, 4,4′-[1,4,10,13-tetraoxa-7,16-diazacyclooctadecane-7,16-diylbis(5-methoxy-6,2-benzofurandiyl)]bis (PBFI), a NO sensitive dye, e.g., 4-amino-5-methylamino-2′,7′-difluorescein, or other fluorophore. In one embodiment, the functional group is an immunogenic molecule, i.e., one which is bound by antibodies specific for that molecule. In some embodiments, substrates herein comprise a cleavable linker, for example, those described in U.S. Pat. No. 10,618,907; incorporated by reference in its entirety.

In some embodiments, ‘dual warhead’ substrates are provided that comprise a haloalkane moiety (e.g., a substrate for a modified dehalogenase (e.g., HALOTAG)) and a dimerization moiety that is a ligand (or capture ligand) for a second binding protein (capture agent). For example, certain embodiments herein utilize a haloalkane linked to a SNAP-tag ligand (Cermakova & Hodges. Molecules 2018, 23(8), 1958; incorporated by reference in its entirety); a haloalkane linked to cTMP (Cermakova & Hodges. Molecules 2018, 23(8), 1958; incorporated by reference in its entirety); a haloalkane linked to rapamycin-like moiety capable of binding to FKBP or FRB (Chen et al. ACS Chem. Biol. 2021, 16, 12, 2808-2815; incorporated by reference in its entirety); or other haloalkane ‘dual warhead’ ligands capable of binding to a modified dehalogenase (e.g., HALOTAG) and a second capture agent. In such embodiments, a system is provided comprising modified dehalogenase described herein, a dual warhead substrate, and a capture agent capable of binding to the dimerization moiety (e.g., FKBP, FRB, SNAP-tag, eDHFR, etc.). In some embodiments, the insert within the modified dehalogenase and the capture agent are capable of interaction (e.g., structurally or by energy transfer). In some embodiments, by adding another protein binding small molecule moiety onto a haloalkane, the dual warheads trigger close proximity of the inserted heterologous sequence and the capture agent. Such embodiments provide forced proximity of the insert and the capture agent. Any suitable linkers may find use in assembly of dual warhead substrates. The linker may include various combinations of such groups to provide linkers having ester (—C(O)O—), amide (—C(O)NH—), carbamate (—NHC(O)O—), urea (—NHC(O)NH—), phenylene (e.g., 1,4-phenylene), straight or branched chain alkylene, and/or oligo- and poly-ethylene glycol (—(CH₂CH₂O)ₓ—) linkages, and the like. In some embodiments, the linker may include 2 or more atoms (e.g., 2-200 atoms, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 atoms, or any range therebetween (e.g., 2-20, 5-10, 15-35, 25-100, etc.)). In some embodiments, the linker includes a combination of oligoethylene glycol linkages and carbamate linkages. In some embodiments, the linker has a formula —O(CH₂CH₂O)_{z1}—C(O)NH—(CH₂CH₂O)_{z2}—C(O)NH—(CH₂)_{z3}—(OCH₂CH₂)_{z4}O—, wherein z1, z2, z3, and z4 are each independently selected form 0, 1, 2, 3, 4, 5, and 6. For example, in some embodiments, the linker has a formula selected from:

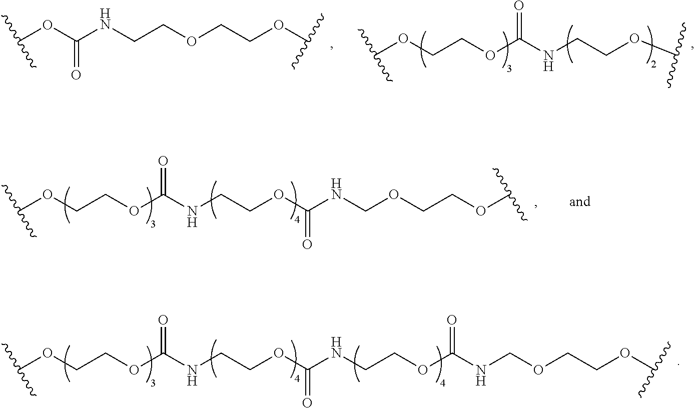

Such linkers may also find use in other tethering, conjugation, and or linking embodiments herein.

In some embodiments, N-terminal cysteine labeling via the condensation reaction between 2-cyanobenzothiazole (CBT) and an engineered N-terminal cysteine residues (e.g., on a reporter protein, target-specific luminescent detection agent, etc.) provides site-specific and stable attachment for lining two components and/or a component to the matrix material.

In some embodiments, light-activated crosslinkers are used to form covalent bonds between components, such as between a target-specific luminescent detection agent and the matrix material. Light-activated crosslinkers provide spatial and temporal control over the immobilization process (e.g., proximity labeling under bioluminescence activation or linkers amenable to UV-dependent crosslinking).

In some embodiments, protein splicing elements, such as inteins, are used to covalently attach proteins (e.g., to the matrix material or other components) through a self-catalyzed process that allows for self-assembling and potentially reversible attachment.

In some embodiments, non-covalent interactions are utilized for conjugation of components (e.g., to other components or to the matrix material). Two moieties with specific affinity for one another, an affinity agent and an affinity ligand, are attached to components to be conjugated (or the matrix material and a component to be conjugated thereto), and the affinity of the affinity agent for the affinity ligand results in conjugation of the components. In some embodiments, because the conjugation relies of non-covalent interactions, addition of excess affinity ligand to the system can result in release of the conjugated components.

In some embodiments, the preferred system for non-covalent conjugation is biotin and streptavidin. The biotin-streptavidin interaction is one of the most widely used non-covalent conjugation pairs. Biotin is a small molecule (vitamin B7) that binds with extraordinarily high affinity to streptavidin (a protein) or avidin (a similar protein found in egg whites). The strength of this interaction makes it highly useful. In some embodiments, an affinity agent (e.g., streptavidin) or a capture agent is linked to the matrix material; addition of an affinity/capture ligand-linked component to the assay particle results in non-covalent, but stable, conjugation of the component to the matrix material. Analogous systems can be used for conjugation of two components together. In the case of an affinity-based conjugation, addition of excess affinity ligand can be used to overwhelm the interaction and release to component from the conjugation. Other pairs for non-covalent conjugation that find use in embodiments herein include GST-Tag/Glutathione (GSH), protein A/immunoglobulin (IgG) (or other antibody-binding agents), antibody (or antibody fragment) and antigen, etc.

In some embodiments, the various covalent/non-covalent, reversible/irreversible, cleavable/non-cleavable, flexible/rigid, of varying lengths, hydrophobicity/hydrophilicity systems for tethering, conjugating, and linking components together and/or to the matrix material find use in constructing assay particles with any combinations of the components described herein for any suitable applications.

The assay particles herein provide biosensors in a discrete reaction space. Integrations of the components described herein (with each other and/or with target analytes, analyte mimetics, etc.) and conformational changes of the components are optimized to provide target-analyte sensitivity. A feature of the embodiments herein is that the components, their connectivity, arrangement, etc., can be varied to provide alternative assay formats for the detection/quantitation of any suitable target and under any desired conditions. The platform is versatile and agnostic regarding the identity of the components that can be used. The use of modular and discrete components, where each assay particle represents one assay, offers precision and versatility. Each assay particle functions independently, allowing for multiple assays to be conducted simultaneously within a single sample. This modularity facilitates high-throughput screening and multiplexing capabilities, making it possible to detect and measure multiple analytes or reactions simultaneously, thereby enhancing the efficiency and speed of analytical processes. In some embodiments, the localization of reactions within individual assay particles ensures that each reaction is confined within the boundaries of its respective bead. This spatial separation minimizes the risk of cross-reactivity between different assays, thereby increasing the reliability and accuracy of the results. It also allows for the detection of multiple targets within a single sample without interference, which is crucial for complex biological and chemical analyses. The spatial separation allows for the luminescent signal from multiple assay particles to be imaged at the same time, thereby reducing time for data collection and analysis.

The conjugation of components (e.g., target-specific luminescent detection agents, etc.) onto the matrix material provides a flexible, application-dependent construction of assay particles. Biosensors are tailored and optimized for specific applications by selecting appropriate components and conjugation/combination thereof. This flexibility allows for the customization of the platform to meet the unique requirements of different assays, ranging from environmental monitoring and pathogen monitoring to clinical diagnostics and beyond. The ability to mix and match assay particles and biosensors within the platform makes it highly transferable and adaptable. Researchers and developers can easily swap out or combine different biosensors to create customized assay panels. This modular approach not only simplifies the development process but also allows for rapid scaling and adaptation to new targets or emerging needs without the need for extensive re-engineering. Adding target analytes and substrates in a homogeneous format streamlines the workflow by eliminating the need for complex and time-consuming sample preparation steps. This homogeneity ensures that all reactions occur uniformly across the assay particles, which can enhance the sensitivity and reproducibility of the assays. It also reduces potential sources of error and variability, leading to more consistent and reliable results.

In some embodiments, the assay-particle-identifying physical characteristic (e.g., spectral signature) and luminescent signal(s) s of the assay particles herein are detectable and/or quantifiable by imaging-based detection. Any imagining system capable of detection of luminescence from discrete assay particles within a sample may find use in embodiments herein. In particular, microscopy imaging is used to detect signals from individual assay particles. In some embodiments, widefield microscopy, confocal microscopy, lifetime imagine microscopy, or another imagine system find use in detecting the luminescence and assay-particle-identifying physical characteristic (e.g., spectral signature) from the assay particles. However, assay readouts are not limited to imaging-based measurements and are compatible with standard plate-readers, for example plate readers with dual luminescence and fluorescence detection capabilities, individual luminometers and fluorescence detectors, etc.

As discussed throughout, the assay particles herein can be configured using various combinations of reporters, analyte interaction elements, etc., to detect any suitable analyte in any suitable sample. The claims and the following assay formats provide exemplary particle configurations but are not limiting on the full scope of the types of assays and assay particles encompassed herein.

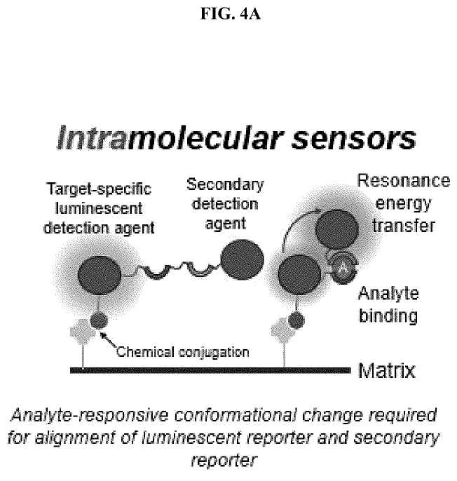

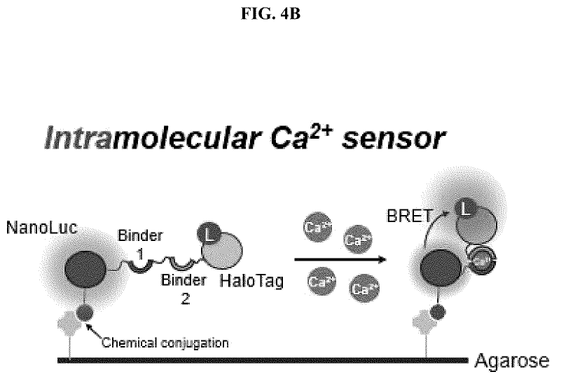

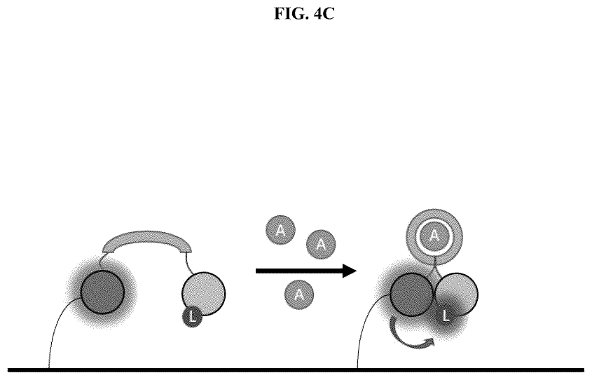

In some embodiments, an assay format is provided for the detection of a target analyte (or multiple different target analytes) using an intramolecular biosensor on and/or within assay particles herein. In an intramolecular sensor, a target-specific luminescent detection agent is provided that comprises two target-specific binding agents and two components of a luminescent reporter. Upon binding of the target-specific binding agents to the target analyte, the components of the luminescent reporter are brought into the proper proximity and/or orientation for emitting a bioluminescent signal (and/or altering (decreasing/increasing), the luminescent signal emitted from the luminescent reporter. The first and second reporter components may interact through resonance energy transfer, as depicted in FIG. 4A, or they may interact through structural complementation (e.g., as in NANOBIT or NANOTRIP). The target-specific binding agents may be any type of binding agents described herein and may be the same or different. The binding agents may both be target specific or one of the two may be a more general target binder. In an exemplary embodiment depicted in FIG. 4B for the detection of calcium ions in a sample, a HALOTAG protein is tethered to a NANOLUC reporter by a pair of Ca²⁺ binding moieties, and the NANOLUC is conjugated to the matrix material; a luminophore substrate for NANOLUC and fluorescent HALOTAG ligand are introduced into the assay particle; in the presence of Ca²⁺, binding of Ca²⁺ by both the Ca²⁺ binding moieties brings the NANOLUC reporter and fluorescent HALOTAG ligand into proximity to allow resonance energy transfer. In such an embodiment, the Ca²⁺ binding moieties may be selected from 1,2-Bis(o-aminophenoxy)ethane-N,N,N′,N′-tetraacetic acid (BAPTA), ethylene glycol tetraacetic acid (EGTA), Calmodulin, Calbindin, Aequorin, etc. In some embodiments, one of the binding moieties is a more generic metal ion binder, such as EDTA, a polyphosphate or citrate, and the other is a specific Ca²⁺ binder. In other embodiments, two different, yet specific, Ca²⁺ binding moieties are used to increase the selectivity for Ca²⁺. Alternative versions of the assay could include specificity for a different ligand (e.g., a different metal ion), a different reporter (e.g., utilizing structural complementation instead of BRET), etc. In some embodiments, a population of assay particles is provided comprising different subsets, each with an intermolecular biosensor, such as those depicted in FIG. 4A-B, capable of detecting a different metal ion (or other ligand). Upon binding exposing the assay particles to a single sample, the various binding moieties in the different subsets of assay particles bind to their specific target analytes, resulting in the detectable signal from that assay particle. Although the particles of each subset emit the same type of luminescent signal upon ligand binding, the particles are differentiable by the assay-particle-identifying physical characteristic (e.g., spectral signature). Therefore, the resulting luminescent signal from each particle can be assigned to a corresponding assay particle subset and therefore the presence/absence/level of a specific analyte in the sample.

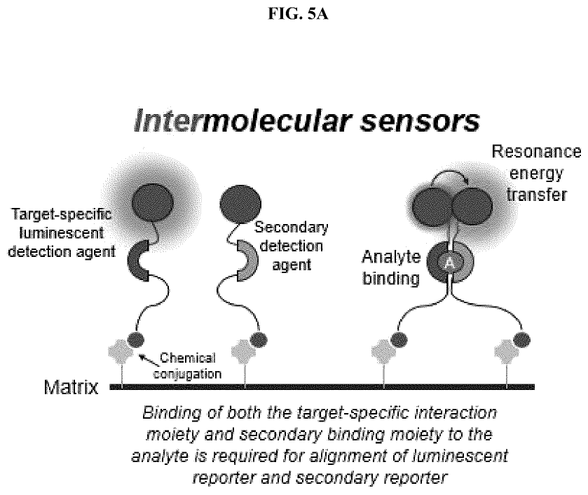

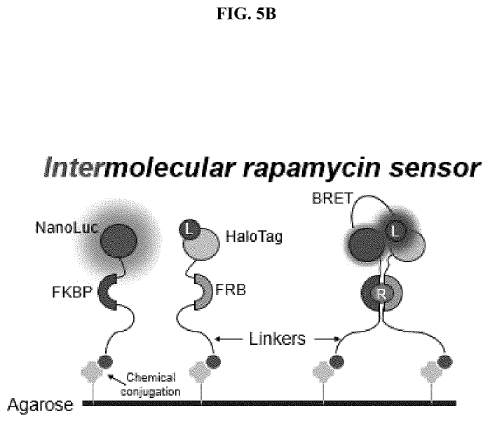

In some embodiments, an assay format is provided for the detection of a target analyte (or multiple different target analytes) using an intermolecular biosensor on and/or within the assay particles herein. In an intermolecular sensor, a pair of target-specific luminescent detection agents are provided that each comprises a target-specific binding agent and a component of a luminescent reporter. Upon bind of the same target analyte by the target-specific binding agents, the components of the luminescent reporter are brought into the proper proximity and/or orientation for emitting a bioluminescent signal (and/or altering (decreasing/increasing) the luminescent signal emitted from the luminescent reporter). The first and second reporter components may interact through resonance energy transfer, as depicted in FIG. 5A, or they may interact through structural complementation (e.g., as in NANOBIT or NANOTRIP). The target-specific binding agents may be any type of binding agents described herein and may be the same or different. The binding agents may both be target specific or one of the two may be a more general target binder. In an exemplary embodiment depicted in FIG. 5B for the detection of rapamycin, NANOLUC is linked to the matrix material via FKBP and HALOTAG is linked to the matrix material via FRB. Upon exposure of the system to a substrate for NANOLUC (e.g., Furimazine) and a HALOTAG ligand comprising a haloalkyl group linked to an appropriate fluorophore, BRET from NANOLUC to the fluorophore can occur in the presence of rapamycin, which results in formation of the FRB/rapamycin/FKBP ternary complex and positions NANOLUC near to the fluorophore. The same intermolecular biosensor could be made specific for a different target analyte by merely using different binding moieties. In some embodiments, a population of assay particles is provided comprising different subsets, each with an intermolecular biosensor, such as those depicted in FIG. 5A-B, capable of detecting a different target analyte. Upon binding exposing the assay particles to a single sample, the various binding moieties in the different subsets of assay particles bind to their specific target analytes, resulting in the detectable signal from that assay particle. Although the particles of each subset emit the same type of luminescent signal upon ligand binding, the particles are differentiated by the assay-particle-identifying physical characteristic (e.g., spectral signature). Therefore, the resulting luminescent signal from each particle can be assigned to a corresponding assay particle subset and therefore the presence/absence/level of a specific analyte in the sample.

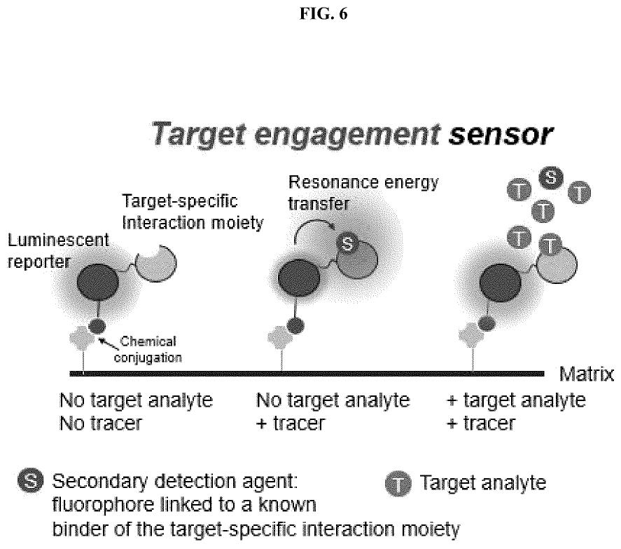

In some embodiments, an assay format is provided for the detection of engagement of a target analyte with a binding partner, such as a target engagement assay. In a target engagement assay, a target mimetic linked to a first component of a reporter is allowed to bind to a protein of interest or other molecular entity of interest (e.g., a binding partner of the target analyte) that is linked to a second component of the reporter; upon exposure of the system to a sample, the target analyte, if present in the sample will compete with the target mimetic for binding to the protein/molecule of interest, thereby reducing a signal produced by the interaction of the first and second components of the reporter. For example, as depicted in the exemplary embodiment of FIG. 6, a target-specific luminescent detection element is provided in which a target-specific interaction moiety is linked to the matrix material via a luminescent reporter; upon binding of a tracer (e.g., target mimetic tethered to a fluorophore) to the target-specific interaction moiety, resonance energy transfer is able to occur between the luminescent reporter and the tracer; upon exposure to a sample containing a target analyte, the target analyte competes with the tracer for binding to the target-specific interaction moiety, thereby reducing the signal produced by the energy transfer. In particular embodiments, a BRET-based (e.g., NANOBRET) reporter system is utilized in which the target-specific interaction moiety is linked to a donor bioluminescent reporter (e.g., a luciferase (e.g., NANOLUC)), and the target mimetic is linked to an acceptor fluorophore. In related embodiments, the first and second reporter components may interact through structural complementation (e.g., as in NANOBIT or NANOTRIP), rather than resonance energy transfer. Various target engagement assays and components for use therewith are described in, for example, U.S. Pat. No. 11,442,066; U.S. Pub. No. 2022/0064696; U.S. Pat. No. 11,327,072; incorporated by reference in their entireties. The target engagement assays that find use with the assay particles may also find use with reporter modalities such as fluorescence anisotropy or thermal shift.

In some embodiments, the assay particle technology herein is utilized to assess the binding of a target analyte to multiple different binding targets (e.g., target-specific interaction moieties) and/or to assess the ability of a target analyte to displace multiple different target mimetics (e.g., tracers) from a binding target. For example, a population of assay particles is provided comprising an identical target-specific interaction moieties and luminescent reporter tethered to the matrix material; different subsets of the population are formed by allowing different tracers comprising a different target mimetics linked to a secondary reporter to bind to the target-specific interaction moiety; each of the subsets emits the same signal based on the association and/or proximity of the luminescent reporter and secondary reporter; upon exposure of the population of assay particles to a sample comprising the target analyte, the target analyte competes with the various target mimetics for binding to the same target-specific interaction moiety in each assay particle; the reduction of signal in a given subset of assay particles indicates displacement of the target mimetic by the target analyte; the identity of the target mimetic is determined by correlation with the known assay-particle-identifying physical characteristic (e.g., spectral signature) for the subset of assay particles. In another exemplary embodiment, a population of assay particles is provided comprising multiple subsets, each comprising a distinct target-specific interaction moiety and identical luminescent reporter tethered to the matrix material; identical tracers comprising a target mimetic linked to a secondary reporter are allowed to bind to the target-specific interaction moiety; each of the subsets emits the same signal based on the association and/or proximity of the luminescent reporter and secondary reporter; upon exposure of the population of assay particles to a sample comprising the target analyte, the target analyte competes target mimetic for binding to the different target-specific interaction moieties in each subset; the reduction of signal in a given subset of assay particles indicates displacement of the target mimetic by the target analyte; the identity of the target-specific interaction moiety is determined by correlation with the known assay-particle-identifying physical characteristic (e.g., spectral signature) for the subset of assay particles.

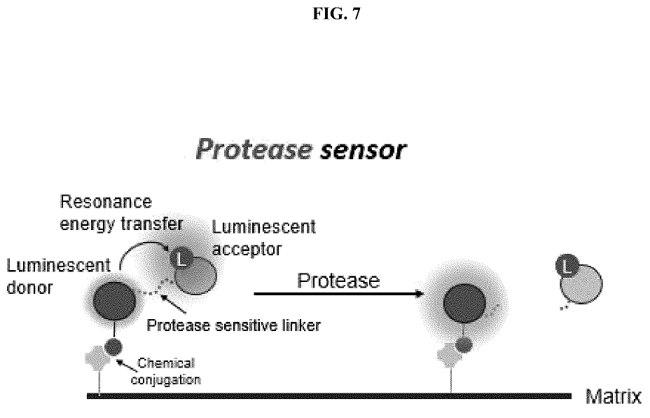

In some embodiments, an assay format is provided for the detection of a cleavage event. In such embodiments, assay particles herein are loaded (e.g., tethered to) a pair of reporters that are connected by a linker that can be cleaved by a target analyte. A signal produced by the reporter pair when linked (e.g., due to resonance energy transfer, structural complementation, etc.) can be detectably differentiated from a signal produced after cleavage of the linker. In an exemplary embodiment depicted in FIG. 7, a luminescent reporter is tethered to the matrix material, a secondary reporter is tethered to the luminescent reporter by a protease-sensitive linker, and the luminescent reporter and secondary reporter are within proximity of each other for resonance energy transfer to occur between the two reporters. Upon exposure to a sample comprising a protease capable of cleaving the linker, the luminescent reporter and secondary reporter are no longer tethered, and resonance energy transfer does not occur. The luminescent reporter and secondary reporter may interact through resonance energy transfer, as depicted in FIG. 7, or they may interact through structural complementation (e.g., as in NANOBIT or NANOTRIP). Embodiments herein are also not limited to protease detection; the linker may be sensitive to any type of molecule (e.g., nucleases, oxygen radicals, metal ions, etc.).

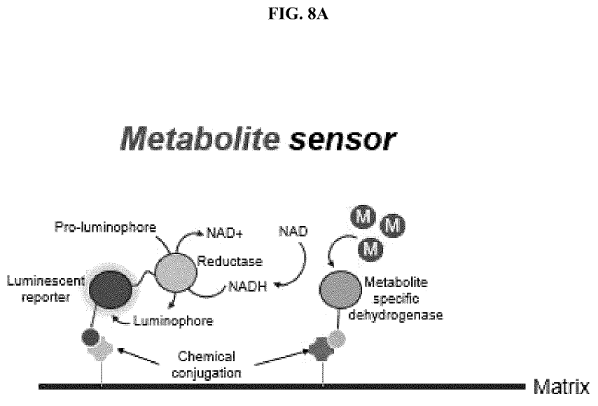

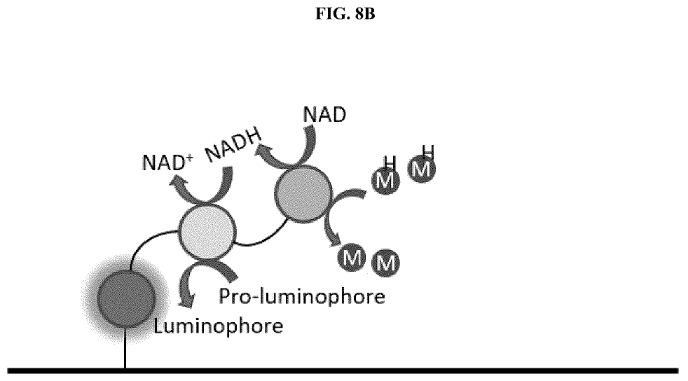

In some embodiments, an assay format is provided for the detection of a metabolite in a sample. In some embodiments, commercially-available assays based on the ULTRA-GLO technology (Promega Corp.), such as Glucose-Glo, Glutamate-Glo, Glutamine-Glo, Lactate-Glo, Malate-Glo, BCAA-Glo, Triglyceride-Glo, etc., are formatted for use in the assay particles and populations of assay particles described herein. The use of such assays is described, for example, in Leippe et al. Bioluminescent Assays for Glucose and Glutamine Metabolism: High-Throughput Screening for Changes in Extracellular and Intracellular Metabolites. SLAS Discov 4, 366-377 (incorporated by reference in its entirety). A generic exemplary embodiment of a metabolite sensing assay that can be performed in the assay particles herein is depicted in FIG. 8. In such embodiments, the presence of a particular metabolite is recognized by a metabolite-specific dehydrogenase. In the presence of the metabolite, the dehydrogenase converts NAD to NADH. A reductase converts a pro-luminophore into an active luminophore substrate in the presence of NADH (NADH is oxidized to NAD⁺). The luminophore then interacts with a luminescent sensor to produce a bioluminescent signal.

In a general embodiment of the metabolite sensor, a metabolite sensing assay particle is provided comprising (a) a metabolite-specific enzyme capable of converting a signal molecule from an inactive to active form in the presence of a metabolite target, wherein the metabolite-specific enzyme is tethered to the matrix material of the assay particle; (b) a luminescent reporter capable of emitting a luminescent signal in the presence of a luminophore substrate, wherein the luminescent reporter is tethered to the matrix material of the assay particle; (c) a signal-molecule-specific enzyme capable of converting an inactive pro-luminophore to the luminophore substrate in the presence of the active form of the signal molecule, wherein the signal-molecule-specific enzyme is tethered to the matrix material of the assay particle (e.g., directly or via the luminescent reporter (e.g., fused to the luminescent reporter)). In the presence of the signal molecule, the pro-luminophore, and the metabolite, a detectable luminescent signal is produced that is proportional to the concentration of the metabolite.

In particular embodiments, provided herein is a metabolite sensing assay particle comprising (a) a metabolite-specific dehydrogenase capable of converting NAD to NADH in the presence of a metabolite target, wherein the metabolite-specific dehydrogenase is tethered to the matrix material of the assay particle; (b) a luminescent reporter capable of emitting a luminescent signal in the presence of a luminophore substrate, wherein the luminescent reporter is tethered to the matrix material of the assay particle; (c) a reductase capable of coupling the oxidation of NADH to NAD⁺ to the reduction of an inactive pro-luminophore to the luminophore substrate, wherein the reductase is tethered to the matrix material of the assay particle (e.g., directly or via the luminescent reporter (e.g., fused to the luminescent reporter)). In the presence of NAD, the pro-luminophore, and the metabolite, a detectable luminescent signal is produced that is proportional to the concentration of the metabolite.

In some embodiments, a population of metabolite sensing assay particles is provided, each comprising the same luminescent reporter and signal-molecule-specific enzyme (e.g., reductase), but different subsets of the population having different metabolite-specific enzymes (e.g., dehydrogenases) that are specific for different metabolites (e.g., as glucose, glutamate, glutamine, lactate, malate, BCAA, triglycerides, etc.). In such embodiments, each subset of assay particles emits the same luminescent signal in response to the presence/concentration of the particular metabolite to which the subset is specific. The identity of the subset to which a particular assay particle belongs (e.g., to which metabolite is it specific) is determined based on the assay-particle-identifying physical characteristic (e.g., spectral signature) that is unique to that subset.

Examples of metabolite-specific dehydrogenases that utilize NAD as a coenzyme and find use in embodiments herein include, but are not limited to glyceraldehyde 3-phosphate dehydrogenase (GAPDH), lactate dehydrogenase (LDH), isocitrate dehydrogenase (IDH), malate dehydrogenase (MDH, pyruvate dehydrogenase complex (PDC), and β-hydroxybutyrate dehydrogenase, etc.

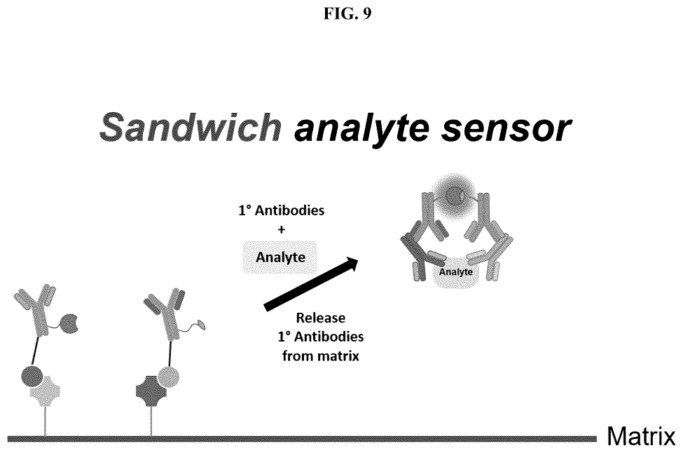

In some embodiments, a sandwich assay analyte sensor format is provided for the detection of an analyte using the assay particles described herein. For example, in certain embodiments assay particles are provided comprising and assay format such as the one depicted in FIG. 9. In such an embodiment, first and second target-specific luminescent detection agents (e.g., antibodies tethered to a component of a luminescent reporter), each capable of binding to the same analyte (e.g., specifically and/or non-specifically), are tethered to the matrix material (one or both is reversibly tethered). Upon addition of a sample comprising the target analyte, one the target-specific luminescent detection agent is capable of binding to the target analyte (the tethering of the target-specific luminescent detection agents to the matrix material would prevent binding of both to the target analyte). Upon release of one or both of the target-specific luminescent detection agents from the matrix material into the interstitial void volume of the assay particle, both target-specific luminescent detection agents are able to bind to the same target analyte, and the components of the luminescent reporter are able to interact to emit a signal the corresponds to the concentration of the analyte in the sample. In some embodiments, the components of the luminescent reporter interact through resonance energy transfer (e.g., BRET, FRET) or structural complementation (e.g., NANOBIT, NANOTRIP).

In some embodiments, a population of sandwich assay particles is provided. The target-specific luminescent detection agents of the assay particles in the population may comprise the same components of the luminescent reporter but target-specific interaction moieties that are specific for different analytes. In such embodiments, assay particles emits the same luminescent signal in response to the presence/concentration of the particular analyte to which the assay particle is specific, and the identity of the assay particle (e.g., to which analyte is it specific) is determined based on the assay-particle-identifying physical characteristic (e.g., spectral signature) that is unique to that assay particle.

In some embodiments, an assay format is provided for multiplex detection of antibodies in a sample using generic reagents (see, e.g., FIG. 10). For example, a sample comprising antibodies to unknown antigens (e.g., different potential pathogens) is contacted with a reagent comprising a generic antibody binding moiety (e.g., protein A, protein G, protein L, etc.) and a luminescent reporter; all suitable antibodies in the sample will then be luminescent labeled. The sample is then contacted with a population of assay particles comprising multiple subsets (e.g., 2, 5, 10, 20, 50, 100, or more), each subset having a known antigen tethered to the matrix material and a assay-particle-identifying physical characteristic (e.g., spectral signature) that is unique to the subset of assay particles displaying a particular antigen. The labeled antibodies capable of binding to one of the displayed antigens will become bound to the particular assay particle. After washing away unbound antibodies, a luminescent signal from an assay particle indicates the present of the corresponding antibody in the sample; the identity of the antigen displayed by a particular assay particle is determined based on the assay-particle-identifying physical characteristic (e.g., spectral signature). In a related embodiment, the antibodies are labeled with a first component of a luminescent reporter, and the second component is linked to the antigen. In such embodiments, a luminescent signal indicating the presence/concentration of the antigen is only produced in assay particles in which the antibody is bound to the antigen (e.g., eliminating the need for washing away unbound antibodies).

In some embodiments, an assay format is provided for multiplex detection of nucleic acids in a sample. In an exemplary embodiment for multiplex detection of nucleic acids, a binary or ternary reporter system is utilized (e.g., binary structural complementation, resonance energy transfer, ternary structural complementation, binary structural complementation+BRET, etc.). In some embodiments, oligonucleotide probes comprising sequences capable of hybridizing to target sequences and a first component of a reporter system are immobilized to the matrix material of an assay particle, either through covalent bonding or other attachment strategies provided herein or understood in the field. In some embodiments, the oligonucleotides are oriented to maximize potential interactions with target nucleic acids. Upon introduction of a sample containing target nucleic acids to the assay particles, the probes will hybridize specifically with complementary sequences in the sample. Addition of the other components of the reporter system forms an active reporter capable of producing luminescent signal that is proportional to the concentration of the target nucleic. In some embodiments the other components of the reporter system are added to the assay particle. In other embodiments the other components of the reporter system are contained within the assay particle and released to interact with the reporter component of the oligonucleotide probe bound to the target nucleic acid. In such embodiments, if the target nucleic acid is present in the sample and hybridizes with the oligonucleotide probe, it will bring the complementary reporter components in proximity to reconstitute the luminescent complex.

In an alternative assay format for detection of nucleic acids in a sample, a first single-stranded nucleic acid (e.g., DNA or RNA) probe and a luminescent reporter (e.g., component of a bioluminescent complex, acceptor fluorophore, donor luciferase, etc.) are tethered to an assay particle; the probe is configured to hybridize to a first region on a target nucleic acid. The assay particles are exposed to a sample that contains (or might contain) the target nucleic acid, and the probe is allowed to bind to the target nucleic acid. Detection reagents are added to the assay particles, the detection reagents comprising a secondary (and possibly ternary) reporter (e.g., component of a bioluminescent complex, acceptor fluorophore, donor luciferase, etc.) attached to a second nucleic acid probe configured to hybridize to a second region adjacent (or near) to the first region on the target nucleic acid. A complex is allowed to form comprising the first and second probes, the target nucleic acid, and the luminescent reporter and the secondary reporter. A signal produced by the interaction (e.g., structural complementation, BRET, FRET, etc.) of the luminescent reporter and the secondary reporter is proportional to the concentration of the target nucleic acid in the sample.

In some embodiments, assay particles and the associated reagents, components, and methods herein find use in detection of analytes (e.g., DNA, RNA, proteins, antibodies, toxins, infectious agents, etc.) in tissue samples (e.g., on slides), providing significant advantages in, for example, sensitivity, multiplexing, and quantitative capabilities, in spatially resolved analyses not found in current methods. In some embodiments, assay particles herein are employed in immunohistochemistry (IHC) or immunofluorescence (IF) techniques/protocols, thereby combining the sensitivity and multiplexing capabilities of the assay particles herein with the spatial resolution of IHC and/or IF. Current methodologies are limited in multiplex capabilities, often relying on visual discrimination by a pathologist and/or use multi-step cumbersome protocols often comprising the tissue sample through the process; the use of the assay particles herein with IHC and/or IF would overcome such limitations.

IHC involves the use of antibodies to detect specific antigens in tissue sections, followed by a colorimetric reaction to visualize the bound antibodies. A typical IHC protocol involves several of the following steps: tissue samples are fixed (often in formalin) and sectioned into slices; nonspecific binding sites may be blocked; primary antibody incubation (e.g., to bind to target analytes); secondary antibody incubation (e.g., to bind labelled antibody to primary antibody); and visualization. IF is similar to IHC but differs in the detection method. Instead of a colorimetric reaction, IF uses fluorescently labeled antibodies to visualize the antigen in tissue samples. Both techniques and variations thereof are understood in the field. An exemplary variation of these techniques using the assay particles herein is provided: tissue is fixed, section, and/or blocked according to existing protocols understood in the field; a population of assay particles containing target-specific luminescent detection agents (e.g., comprising a target-specific interaction moiety, and a luminescent reporter) are added to the tissue (different subsets of assay particles within the population comprise different target-specific interaction moieties capable of binding different analyte; the assay particles of the different subsets are differentiated by different assay-particle-identifying physical characteristics (e.g., spectral signatures)); free secondary detection agents (e.g., comprising a secondary target-specific interaction moiety and a secondary reporter) are added to the tissue; upon binding of the target-specific interaction moiety and the secondary target-specific interaction moiety to the same analyte, interaction of the luminescent reporter and secondary reporter produces a detectable signal; the detectable signal is detected; the assay-particle-identifying physical characteristics (e.g., spectral signatures) of the assay particles are detected; and the spatially-resolved identify of the analytes are identified throughout the tissue sample. Such applications can find use with any combinations of the assay particles and components thereof described herein (e.g., structural complementation, BRET, FRET, etc.).

In some embodiments, the assay particles herein find use in single cell analysis of analytes, for example, when paired with microfluidic chips. Microfluidic chips offer a powerful and precise way to isolate, manipulate, and analyze individual cells in a high-throughput, highly controlled environment. Microfluidic chips leverage micro-scale channels, valves, and chambers, to perform analyses on single cells while consuming minimal reagents and sample volumes. Mirofluidic chips process large numbers of single cells simultaneously and allow for single-cell genomics, transcriptomics, proteomics, and other assays, where cellular heterogeneity is to be captured. The components (e.g., channels, valves, chambers, actuators, sensors, etc.) and protocols for microfluidic separation of single cells is understood in the field and finds use in embodiments herein. For example, a sample comprising a cell population is applied to a microfluidic chip (or technology capable of separating single cells for parallel analysis) and single cells are separated into chambers containing a population of different assay particles, target-specific luminescent detection agents, substrates, etc. (or assay particles and other reagents are added after cell isolation). Analysis using any of the various suitable assays described herein allows for multiplex analysis of analytes of on a single cell to be performed in parallel for multiple single cells from a sample. In some embodiments, an assay format is provided for use of the assay particles herein for multiplex flow cytometry. Assay particles are functionalized (e.g., via a suitable conjugation technique) with target-specific interaction moieties capable of binding to cell surface markers that are unique to a specific cell type. A population of assay particles may comprise multiple subsets of assay particles, each displaying target-specific interaction moieties capable of binding to cell surface markers of different cell types. Each subset also contains an assay-particle-identifying physical characteristic (e.g., spectral signature (e.g., fluorescent signature)) that is unique to the subset. A sample containing one or more cell types is combined with the population of assay particles under conditions that allow the assay particles to bind to their specific cell type (if present in the sample). Flow cytometry is then used to determine the identity of the cells present in the sample.

In a particular embodiment, assay particles herein are used in cellular flow cytometry to characterize, for example, peripheral blood mononuclear cells (PBMCs) in a sample, to provide detailed insights into the cellular composition and functionality of the immune cells. The assay particles are encoded with a specific assay-particle-identifying physical characteristic (e.g., spectral signature) and functionalized with antibodies of ligands that bind to cell surface markers unique to various types of PBMCs and identification of various immune cell types within PBMCs, such as lymphocytes (T cells, B cells, NK cells) and monocytes. The PBMCs are isolated from blood samples using, for example, density gradient centrifugation, which separates them from other blood components. The isolated PBMCs are incubated with the encoded assay particles. During this incubation, the assay particles bind to their corresponding cell markers due to the specificity of the antibodies or ligands on their surface. The specific fluorescent encoding of the assay particles allows for detailed analysis of the cell surface markers, providing insights into the cellular composition of the PBMCs. In other embodiments, assay particles are designed/fabricated to assess cellular functions or states, such as activation markers, cytokine production, or other immunological functions, rather than cell type.

As discussed herein, assay particles can be lyophilized and added to a liquid sample (e.g., suspected of comprising one or more analytes) for reconstitution. While lyophilized assay particles can find use with any assay formats described herein or consistent with the embodiments herein, certain embodiments may find particular benefit from lyophilized particles. For example, lyophilized assay particles may be contained within a storage device or within the flow path of a liquid sample. Upon contacting the sample, the lyophilized assay particles are reconstituted within the sample, and the components of the assay particles can interact with target analytes that may be present in the sample. The assay particles can then be recovered (e.g., by magnetic separation, by filtration, etc.) and analyzed to determine the presence/concentration of analytes within the sample.

In some embodiments, lyophilized assay particles find use in methods to query large sample volumes. In particular exemplary embodiments, assay lyophilized assay particles are included in a urine collection bag, the interior of a catheter, IV tubing, etc. As the sample (e.g., urine, blood, etc.) moves through or is contained within the vessel, the assay particles are resuspended. The assay particles are then isolated/collected (e.g., by magnetic separation, by filtration, etc.), and the assay particles are analyzed. In the example of a urine collection bag, as the bag is emptied, the drainage tube can have a magnetic sampling port to capture the assay particles, and the particles are analyzed in a hospital lab for presence of infections/biomarkers. Such an embodiment could be used to detect urinary tract infections, biomarkers to assess kidney function, etc. Other embodiments herein could use lyophilized assay particles and other assay formats to analyze other samples in various contexts.

The various assays, methods, materials, components, reporters, interaction moieties, assay-particle-identifying physical characteristics, linkers, conjugation techniques, and other elements, steps, etc., described above can be combined in any suitable manner using the understanding in the field to achieve any assay particles, populations of assay particles, and assays that can be contemplated based thereon.

In some embodiments, the compositions and methods herein are utilized to perform multiplex assays (e.g., for the detection of multiple analytes in a single sample). The assay particles herein provide a platform that is well suited to multiplex assays. In some embodiments, assay multiplexing further comprises computational methods, algorithms, and/or machine learning allows researchers to extract more information and/or increase throughput. In multiplexed assays, multiple analytes may be detected/quantitated at once, often generating large and complex datasets. In some embodiments, provided herein are computational methods for use with the assay particles herein to integrate and analyze the data (e.g., correlate assay-particle-identifying physical characteristic, identity of analyte, size of luminescent signal, concentration of analyte, etc.). Multiplexed assays can result in data with many variables and/or measurements; in some embodiments, dimensionality reduction methods (e.g., principal component analysis (PCA) or t-SNE (t-distributed stochastic neighbor embedding) are used in embodiments herein for transforming data into a lower-dimensional space, making it easier to visualize, interpret, and process. In some embodiments, machine learning algorithms, such as supervised learning (e.g., decision trees, random forests, support vector machines), unsupervised learning (e.g., clustering), and deep learning are employed to recognize patterns in multiplexed data. In some embodiments, methods herein utilize statistical modeling and multivariate analysis to analyze data collected from multiplexed assays (e.g., interactions between multiple analytes can occur, and statistical models can help understand these relationships, multivariate statistical methods can be employed to analyze how different analytes co-vary or correlate with each other, etc.). In some embodiments, bioinformatics algorithms are used for data normalization and correction. Other methods that may find use in embodiments herein include error detection and quality control by using machine learning to flag outlier data points or problematic data, predictive modeling, real-time data processing, etc.

### EXPERIMENTAL

### Example 1

### Porous Agarose Assay Particles Loaded with Bioluminescent Reporters

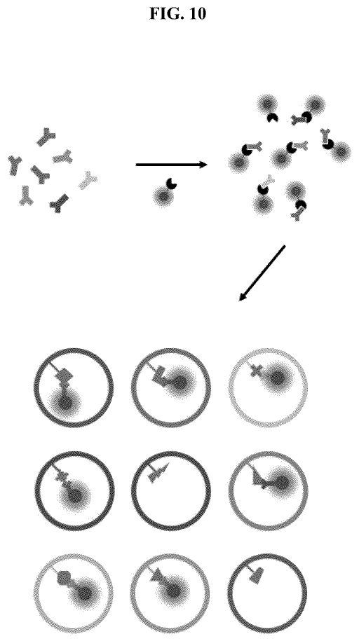

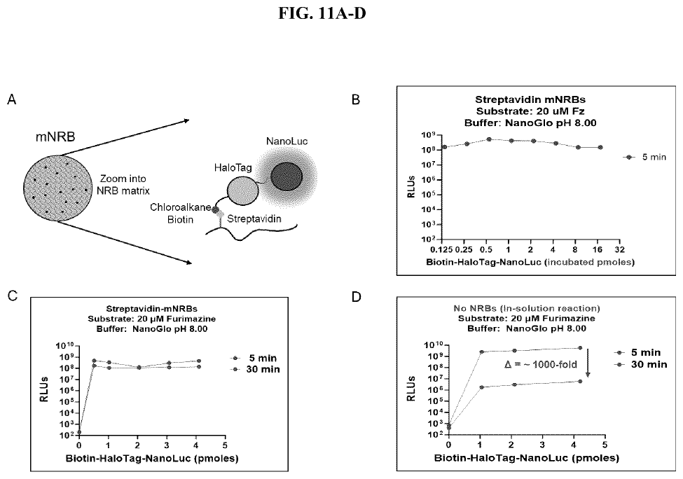

Porous agarose assay particles provide a favorable environment for robust and stable luminescence signal (FIG. 11). The assay particles were functionalized with streptavidin allowing for subsequent binding of biotinylated moieties. Inventory HaloTag-NanoLuc was biotinylated using a chloroalkane-biotin ligand and tethered onto the streptavidin assay particles (FIG. 11A). On performing a titration with this fusion protein, it was observed that low amounts of luciferase (FIG. 11B) generate a robust luminescence signal, which reaches saturation quickly. The luminescent signal remains stable over time when generated on the assay particles (FIG. 11C), in stark contrast to the 1000-fold decreased signal for the same experiment carried out in free solution in the absence of the assay particles (FIG. 11D). This indicates that the unique microenvironment of the mNRBs overcomes unfavorable conditions for NanoLuc and its cognate substrate Furimazine, such as high pH. Furthermore, the experiments indicate that an luciferase/assay particle platform is less sensitive to environmental variations.

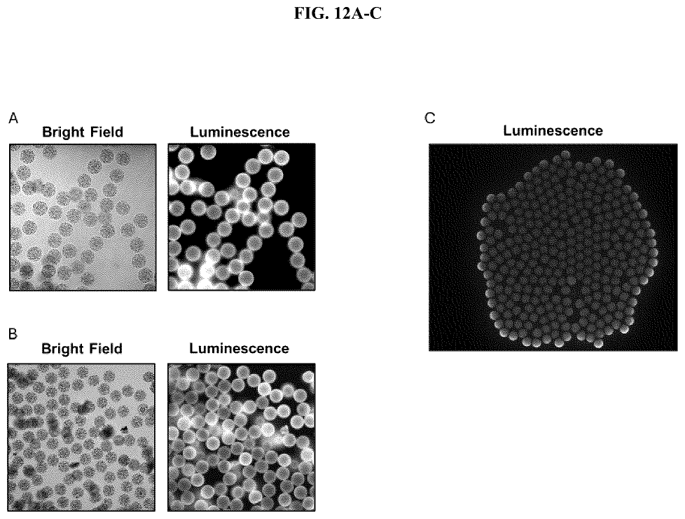

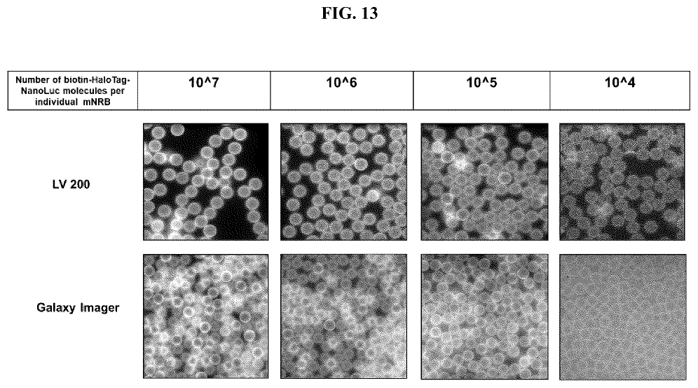

The bioluminescent signal generated from NanoLuc bound to porous agarose assay particles allows for individual bead discrimination and are easily identified against background (FIG. 12), allowing for individual bead analysis. Luciferase-dependent signal from the assay particles was be captured using different imagers with differing sensitivities and performance (FIGS. 12A, 12B, and 12C), displaying the robust nature of this platform. This platform can be used to image signal under steady state conditions (FIGS. 12A and B) in which Furimazine at 20 μM freely diffuses into the assay particles. It is also amenable to dynamic-flow conditions (FIG. 12C) in which Furimazine at 20 μM is made to flow through the assay particles at a constant rate of 100 μL/minute, and the assay particles are immobilized using a magnet within a microfluidic device. This broad versatility shows compatibility with different instruments and formats, and thereby provides a flexible system for users.

The optimal amount of NanoLuc in the exemplary assay particle system was empirically determined to be 75 attomoles (i.e., ~4.5×10⁷ molecules per individual assay particle). Assay particles can reliably be imaged, and individual assay particles can be discriminated to at least 10⁴ molecules per assay particle (i.e., 3-4 log fold below optimal loading density of NanoLuc® molecules). This displays a wide dynamic range based on imaging and demonstrates that this sensitivity can be leveraged to titrate luciferase-based biosensors.

Imaging-based measurements follow similar quantitative trends as in plate-based assays (FIG. 14). Using free solution experiments at steady state conditions, the bioluminescence signal intensity is converted to grey values post imaging for known standard concentrations of NanoLuc, and a calibration curve is established (FIGS. 14A and B). Using this curve, it is possible to calculate the number of biotin-HaloTag-NanoLuc molecules accurately per μL that are incubated with assay particles based on specific luminescence signal originating from the assay particles (FIG. 14C).

### Example 2

### Incorporation of a Spectral Signature and Bioluminescent Detection into Exemplary Assay Particles

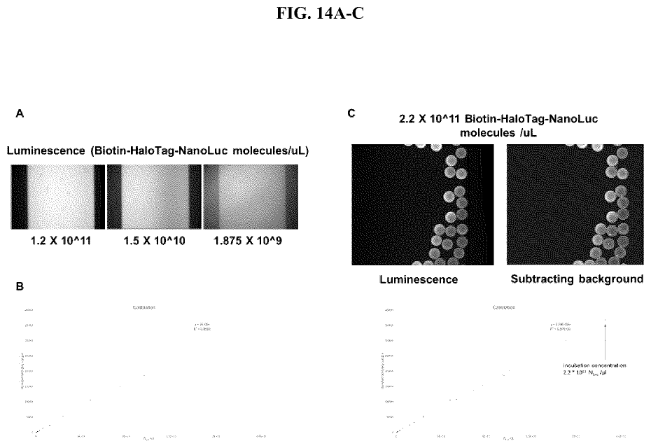

Assay particles were fluorescently encoded with a combination of four fluorescent dyes, producing four distinct subsets of the population of assay particles (FIG. 15). Each subset is represented by a single spectral signature. When combined with a luciferase, the bioluminescent signal can be separately imaged from each of the four fluorescence channels employed to generate unique spectral signatures as indicated in FIG. 15B. When the fluorescence channels are overlayed together, the luminescence signal of each assay particles can be correlated with the spectral signature with which it is labeled.

### Example 3

### Complementation-Based Bioluminescence on Exemplary Assay Particles

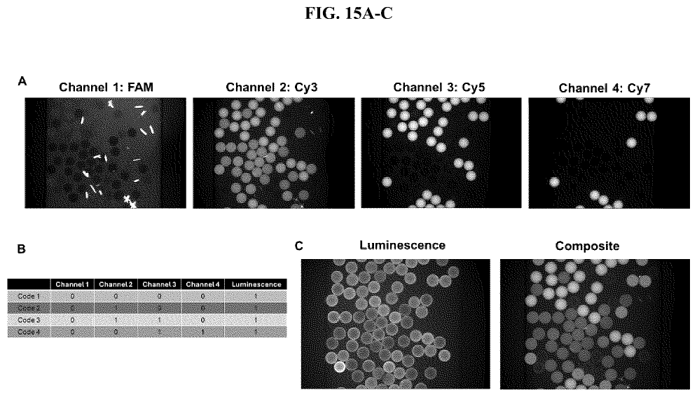

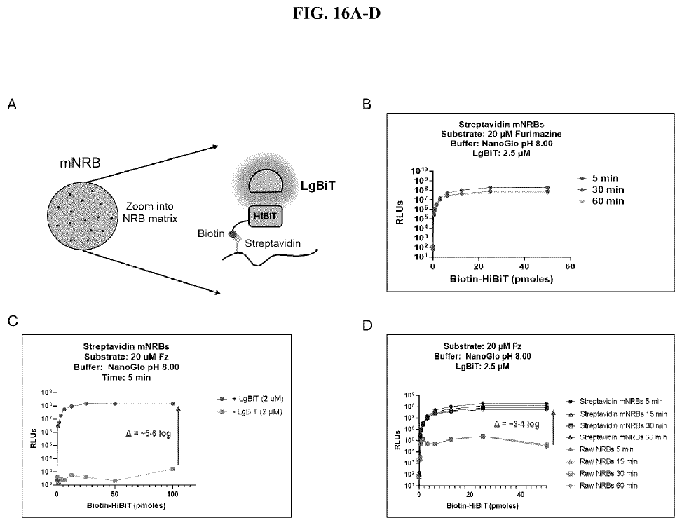

Experiments were conducted during development of embodiments herein to demonstrate that an exemplary porous agarose assay particle system is compatible not only with a luciferase reporter, but also with a binary complementation system (NANOBIT). A biotinylated HiBiT peptide was allowed to bind to streptavidin-functionalized porous agarose assay particles, which were subsequently incubated with LgBiT to generate the complemented NANOBIT complex (FIG. 16A). Similar to full-length NanoLuc, the NanoBiT system produced robust and stable bioluminescent signal in the mNRBs over time, with minimal signal loss at 60 minutes (FIG. 16B). The assay particles were immobilized using magnetic manipulations via magnetic particles within the assay particles and the assay particles were washed multiple times to allow for stringent removal of excess LgBiT, which significantly improved signal-to-noise and provided a wide dynamic range (FIGS. 16C and D).

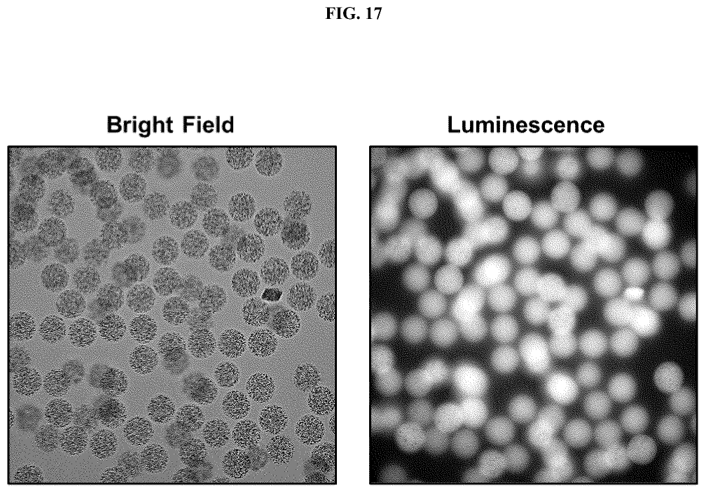

The bioluminescence from NANOBIT was detected over background using the GloMax® Galaxy imager (FIG. 17), demonstrating that the microenvironment of the assay particles within the interstitial void volume allows for geometries favorable for complementation and doesn't pose steric hindrances.

### Example 4

### Complementation-Based BRET on Exemplary Assay Particles

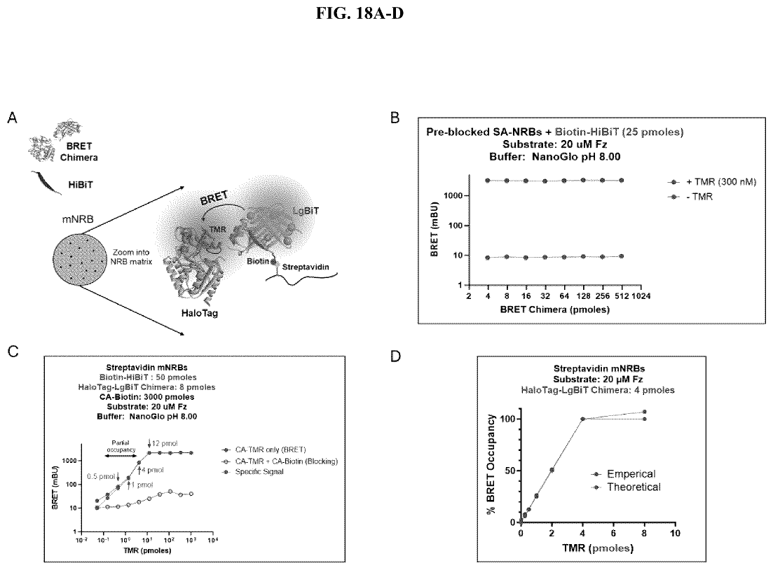

Assay particles herein are compatible not only with complementation-based bioluminescence, but also with energy transfer to a bound fluorescent acceptor (FIG. 18). Experiments conducted during development of embodiments herein demonstrate that more complex complementation-based BRET systems still provide quantitative analyses with the exemplary assay particles. Streptavidin-functionalized porous agarose assay particles were saturated with biotinylated HiBiT, followed by LgBiT driven complementation of a cpLgBiT chimera that was pre-labeled with an acceptor fluorophore (FIG. 18A). The equivalent BRET ratio for over 3 orders of magnitude of cpLgBiT chimera concentrations demonstrates the main advantage of ratiometric BRET readouts (FIG. 18B). Despite unequal loading of biosensors, the BRET output collapses this variable dimension to give consistent readouts. The specificity of the BRET signal is further demonstrated through consistent readouts even at high concentrations of the fluorescent acceptor (TMR), which can be effectively washed away, improving signal to noise (FIG. 18C). It was also observed that the assay particles provide quantitative BRET as incubating with TMR at sub-saturating levels generates BRET signal that strongly coincides with the theoretically predicted relative signal (FIG. 18D). Experiments conducted during development of embodiments herein demonstrate discrimination between partial occupancies of acceptor fluorophores and is compatible with BRET assays which depend on this principle, such as compound profiling for target engagement.

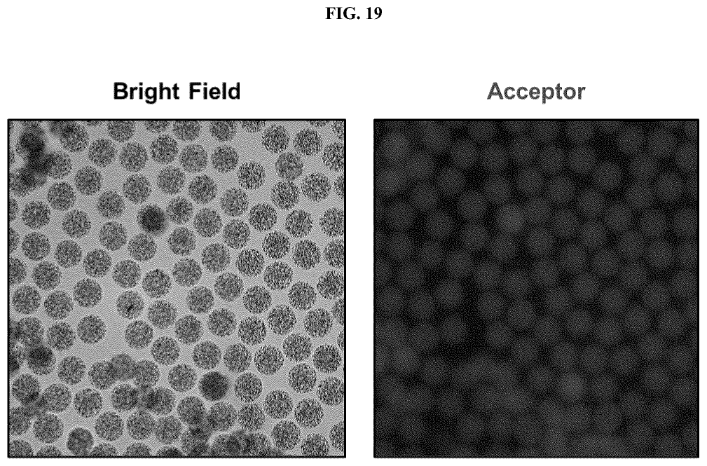

The BRET signal could be detected over background using the GloMax® Galaxy imager (FIG. 19). The microenvironment of the assay particles within the interstitial void volume allows for geometries favorable for complementation and energy transfer to a bound acceptor fluorophore. Experiments conducted during development of embodiments herein provide robust evidence that biosensor systems dependent on complementation-based luciferases as donors and fluorophores as acceptors will have steric considerations amenable for quantitative BRET using the assay particles described herein.

### Example 5

### Dispersion in Non-Aqueous Medium

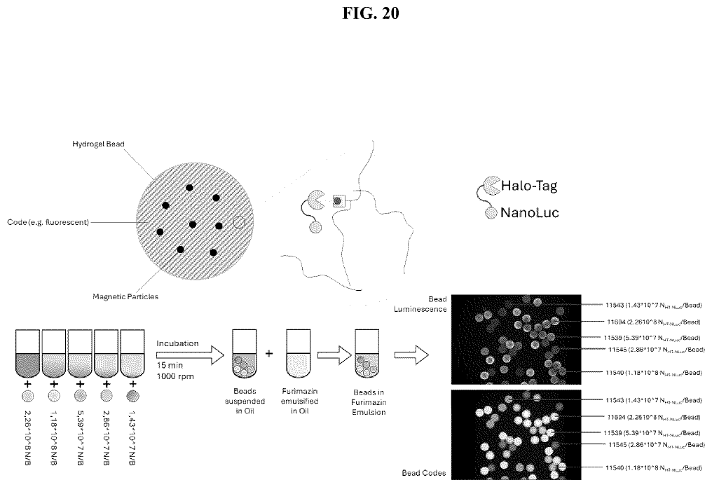

Streptavidin-functionalized porous agarose assay particles were loaded with different amounts of biotinylated NANOLUC enzyme, mixed, and suspended in silicone oil. A 100 μM Furimazine-solution was emulsified in silicone oil. The emulsion was mixed with the bead suspension and imaged through a 10× Objective Lens on a Zeiss Observer Microscope equipped with a Retiga E7 camera. Five dilutions of NANOLUC were prepared in NanoGlo® buffer. Five different subsets of assay particles (10.000 nanoreactors each in 20 μL of bead slurry) carrying using spectral signatures (fluorescent codes) were incubated individually with the different dilutions of enzyme for 15 min at 1,000 rpm. Following two wash steps with NanoGlo® buffer, 5 μL of each bead slurry were mixed and suspended in beads mixed with 175 μl of silicone oil+10% ES5300. A magnetic rack was used to remove the supernatant and perform an additional wash step with 175 μl silicone oil+10% ES5300. In parallel, a 50 μM Furimazine suspension was prepared with 10% ES5300 in silicone oil and added to a bead solution suspension in oil containing app 5000 beads. Imaging was done with an IBIDI chamber on a Zeiss Observer Microscope equipped with fluorescence filters for code imaging and an open channel for luminescence imaging. A 10× magnification lens was used in combination with an Retiga E7 camera (EDR mode, 4×4 binning, 50 sec exposure time for luminescence). The experimental setup and imaging results are depicted in FIG. 20.

### Example 6

### Compartmentalization for Multiplexing

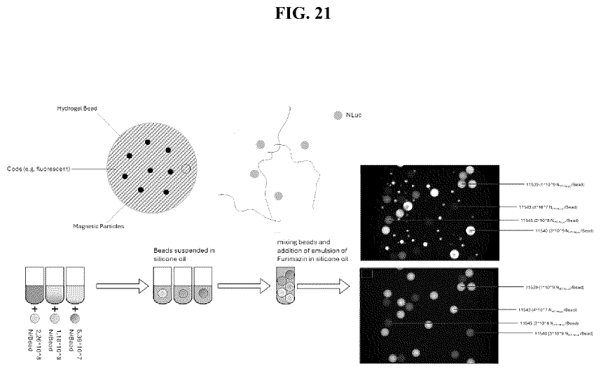

Compartmentalization of different samples in assay particles was demonstrated by using subsets of a population of porous agarose assay particles with different spectral signatures as 0.38 nL-sized compartments for simultaneous detection of different concentrations of luciferase in solution (may serve as model for any type of homogenous assay). Three dilutions of NanoLuc (Nluc) were prepared in NanoGlo® buffer. Three different assay particle types carrying an unique spectral signatures (10,000 particles each in 20 μL of bead slurry) were incubated individually with different dilutions of enzyme for 5 min at 1,000 rpm. 5 μL of each bead slurry were mixed individually with 175 μl of silicone oil+10% ES5300. A magnetic rack was used to remove the supernatant. In parallel, a 50 μM Furimazine suspension were prepared with 10% ES5300 in silicone oil and added to the beads. Following 10 aspiration/dispersion steps for proper suspension, imaging was performed with an IBIDI chamber on a Zeiss Observer Microscope equipped with fluorescence filters for code imaging and an open channel for luminescence imaging. A 10× magnification lens was used in combination with an Retiga E7 camera (EDR mode, 4×4 binning, 50 sec exposure time for luminescence). The experimental setup and imaging results are depicted in FIG. 21.

### Example 7

### Dehydrated Assay Particles

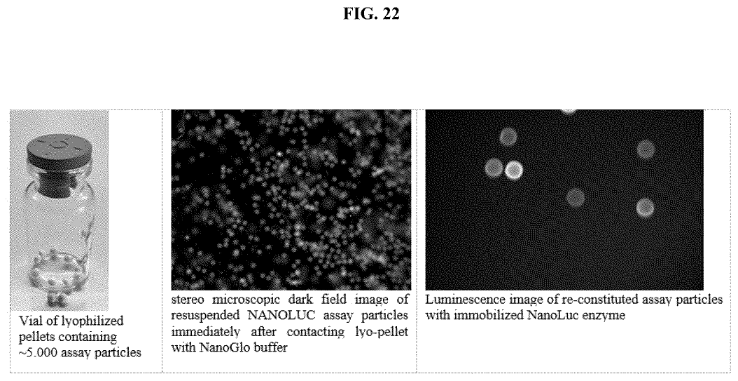

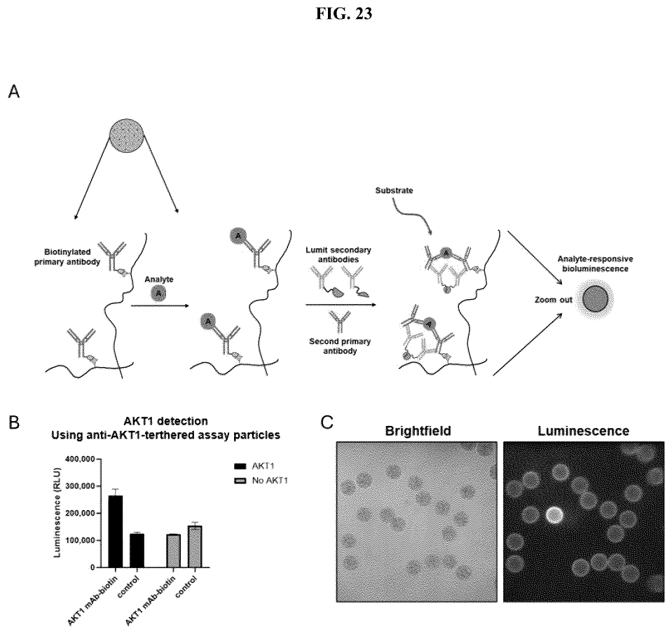

Porous agarose assay particles with immobilized NANOLUC enzyme were lyophilized to produce lyophilized assay particle pellets containing approximately 5,000 assay particles per pellet. The pellets were re-suspended in NanoGlo® buffer and mixed with the same volume of 50 μM Furimazine in NanoGlo® buffer (FIG. 22).

### Example 8

### Transport of Furimazine Through a Non-Aqueous Matrix

Assay particles were prepared comprising NanoLuc® luciferase. The assay particles were dispersed in a non-aqueous emulsion (comprising PDMS oil and 10% ES5300 silicone emulsifier). Experiments were conducted to attempt to add the NanoLuc® substrate Furimazine to the assay particles through the emulsion. It was found that Furimazine is not soluble the emulsions to a sufficient extent to allow the Furimazine to pass through the emulsion and provide for a substrate concentration of Furimazine within the assay particles sufficient to generate a luminescent signal within the assay particles. Experiments were then conducted to emulsify the Furimazine in a carrier (e.g., similar to the non-aqueous emulsion of the assay particles) for adding to the assay particles.

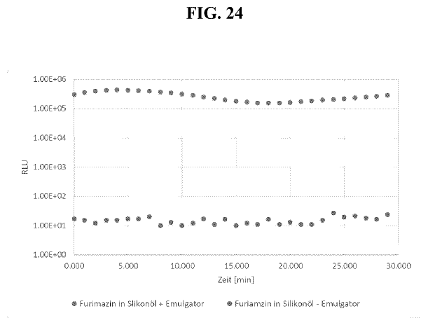

For detection, 10,000 assay particles were incubated with 1×10¹¹ molecules of NanoLuc. After an incubation of 30 min at 1500 rpm on an orbital shaker, the assay particles were washed three times with storage buffer. Subsequently, the NanoLuc®-laden assay particles were transferred into silicone oil with 10% ES5300 Silicone emulsifier. In each case, 80 μl of the emulsified NanoLuc®-laden assay particles were pipetted into one well of a 96-well MTP. Two preparations of Furimazine were prepared. The first contained 1 μl Furimazine in 49 μl silicone oil with 10% ES5300 Silicone emulsifier. The second contained 1 μl Furimazine in 49 μl silicone oil without emulsifier. Both were shaken with a vertical shaker at 3000 rpm for 180 sec. For measurements, the two Furimazine preparations were each added to a well containing the emulsified NanoLuc®-laden assay particles. The plate was agitated for 20 sec at 400 rpm in a GloMax® device. The luminescence was then detected with an integration time of 2 sec (FIG. 24). Luminescence for the preparation without emulsifier is equivalent to background signal.

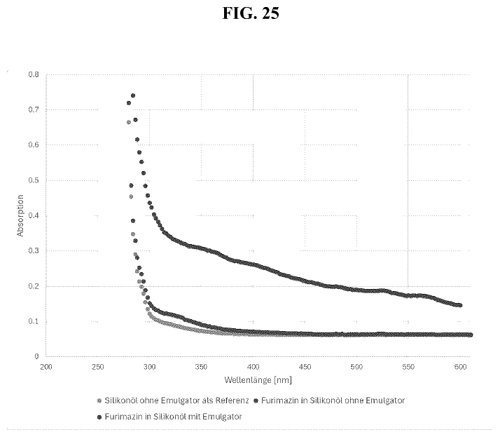

To investigate solubility of Furimazine in silicone oil with and without emulsifier, an absorption spectrum of silicone oil, of Furimazine (Fz) in silicone oil without emulsifier, and of Furimazine in silicone oil with ES5300 Silicone emulsifier (10%) was obtained. The data is summarized in the FIG. 25. Whereas absorbance values without emulsifier are similar to background absorbance by silicon oil, the preparation with emulsifier shows a distinct absorbance profile.

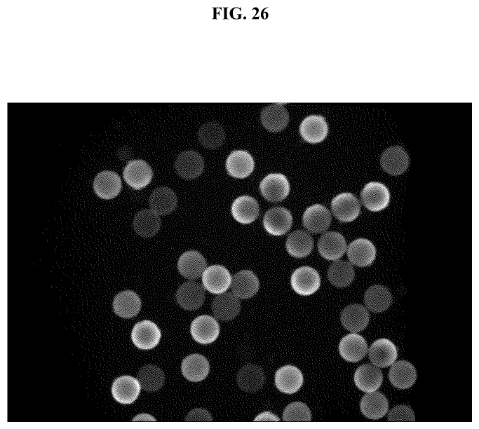

Assay: Five differently encoded Streptavidin coated assay particles were incubated with five different concentrations of biotinylated NanoLuc® luciferase, washed with Nano-Glo® buffer, mixed, and suspended in silicone oil with emulsifier. The assay particle oil suspension was then mixed with a 50 μM Furimazine emulsion (silicone oil+10% ES5300 Silicone emulsifier). The suspension was then transferred to a slide and imaged on a microscope (20× magnification with the Retiga E7 camera for 1 minute (16× binning)). An exemplary image obtained (FIG. 26) depicts different levels of luminescence from assay particles containing different concentrations of NanoLuc and carrying different codes. The detected luminescence intensity can be correlated to the adjusted amount of luciferase on each assay particle based on its individual code.

### Example 9

### Transport of Biotin-Oligonucleotide Conjugate Through Non-Aqueous Medium

Streptavidin was bound to assay particles comprising 0.1% biotinylated agarose in 250 mM TrisHCl pH 8.3 containing 4M NaCl buffer. Beads were washed 4× using 50 mM TrisHCl pH 8.3 followed by a washing step with H₂O. 20 μL of the assay particles were then incubated for 5 min in 40 μL of 5 mM Tris-HCl pH 8.5 or 40 μL H2O including 0.1% Tergitol or 20 mM Bis-Tris-Propane pH 6.8 including 0.1% Tergitol or 100 mM Tris HCl pH 8.8 including 22 mM NH₄Cl and 22 mM KCl. Bead supernatant was removed, and beads were emulsified using 100 μL 2 cST PDMS oil containing 10% ES5300 as emulsifier by pipetting the bead/oil mix up/down for 30×. The microemulsion was removed, and beads were washed with 200 μL 2 cST PDMS oil containing 10% ES5300.

0.5 μM of a 23 nt oligonucleotide with Cy5 label at 5′ and biotin at 3′ terminus were mixed with 10%, 20% and 40% Tergitol, or with 10% LDAO. 5 μL of each solution was then emulsified with 20 μL 2 cST PDMS oil containing 10% ES5300 by pipetting the mix up/down for 30×.

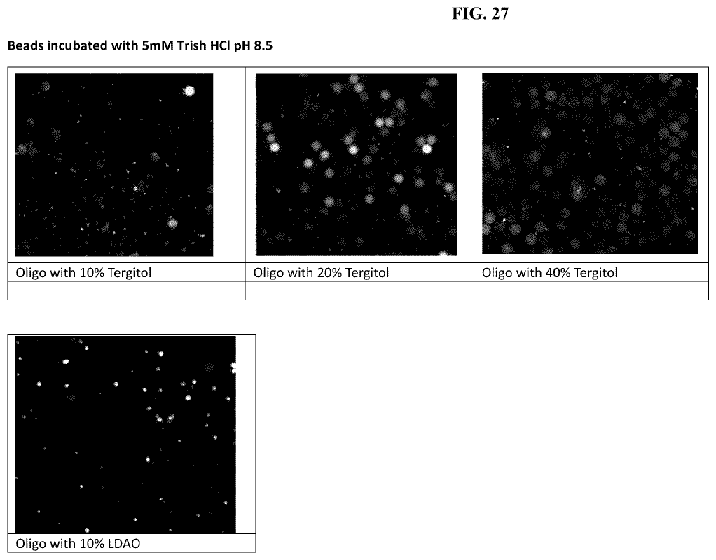

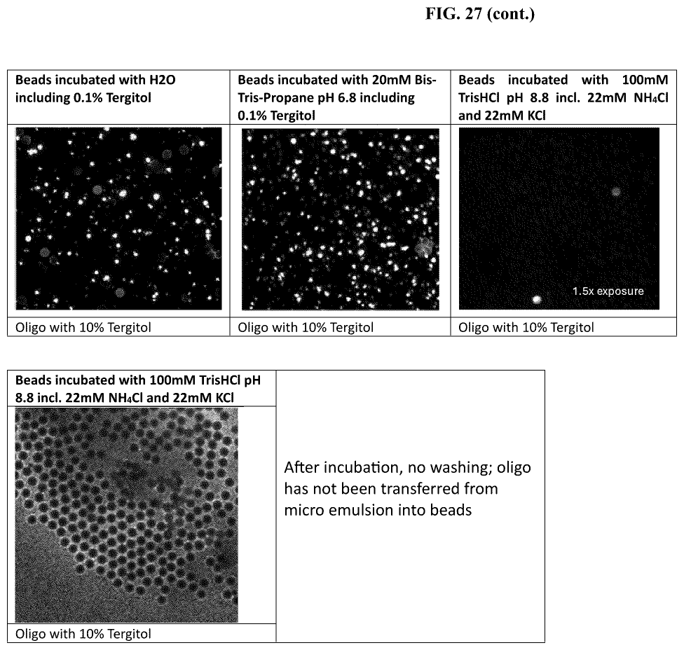

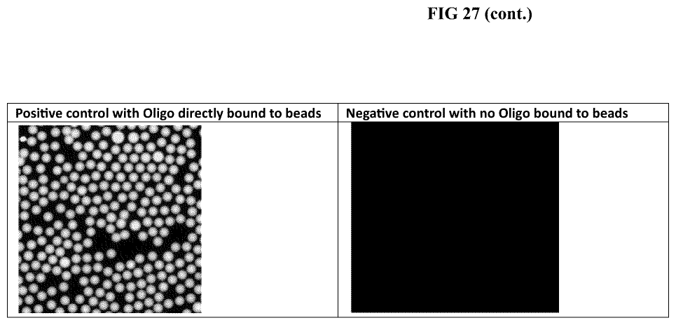

120 μL bead suspension and 25 μL of oligo emulsion were mixed using a pipette (30× up/down) and incubated for 5 min at 25° C. at 1500 rpm on a thermoshaker. The supernatant was then removed, and beads were washed with 200 μl of suspension oil. Beads were transferred onto a Blink X plate, and images were obtained (FIG. 27). The results clearly demonstrate that different buffer compositions (e.g., detergent concentration, pH, salt) for the assay particles and the ligand emulsion affect the transferability of the ligand from the emulsion to the assay particles. The results also demonstrate that even highly hydrophilic compounds, such as biotinylated, dye-modified oligonucleotides, can be successfully transferred into oil/emulsion-dispersed assay particles using an appropriate carrier, and that materials not in a suitable carrier will not enter the assay particles.

### Example 10

### Homogenous Fully Enclosed Particle-Tethered Protein Detection System

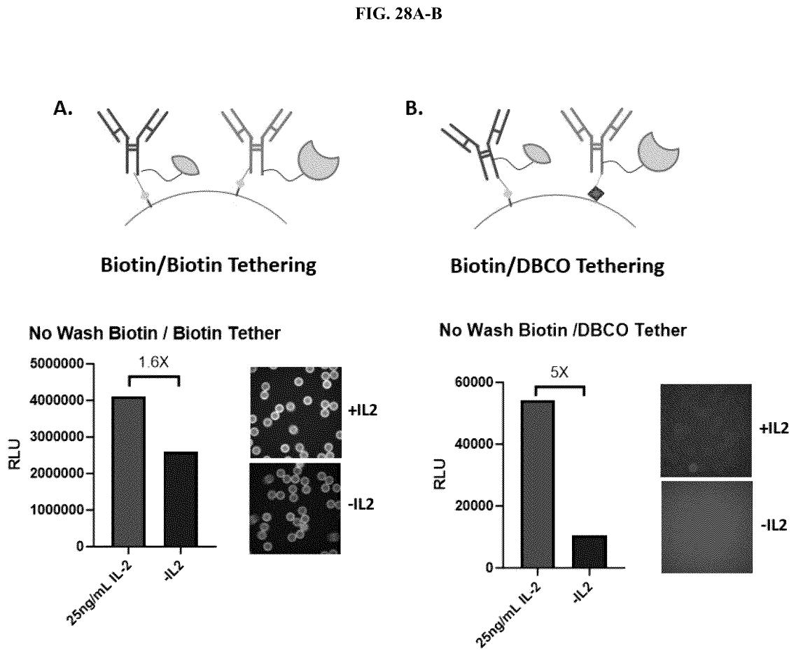

Two distinct IL-2 antibodies were labeled with biotin and either the SmBiT or LgBiT portion of the NanoBiT® luminescent reporter system (Promega). The antibodies were bound to reaction particles via streptavidin (FIG. 28A). Similarly, IL-2 antibody labeled with SmBiT was labeled with biotin, while a distinct IL-2 antibody labeled with LgBiT was labeled with DBCO to bind orthogonally to the reaction particles via streptavidin and azide respectively (FIG. 28B). In both cases, particles were prepared by washing off unbound antibody 3 times for 30 minutes total. In the presence of analyte, the two antibodies are designed to come together and produce a luminescent signal. During the protein detection process, labeled beads were simply incubated in the presence or absence of analyte, with no washes required. After 1.5 hrs, Furimazine substrate (Promega) was added, and luminescent signal was imaged and read on a plate reader. The Biotin/Biotin tether and Biotin/DBCO tether showed 1.6 or 5-fold increase in signal, respectively, in the presence of IL-2 showing a homogenous protein detection system that can be fully enclosed within a bead to avoid cross-reactivity with other binders in a multiplex system.

### Example 11

### Homogenous Two Antibody Protein Detection System with a Single Antibody Tethered to an Assay Particle

One antibody specific to the analyte of interest was orthogonally labeled with biotin and labeled with either the SmBiT (FIG. 29A) or LgBiT (FIG. 29B) portion of the NanoBiT® luminescent reporter systems. The antibody was tethered to the reaction particle via biotin-streptavidin. Reaction particles were prepared by washing off unbound antibody 3 times for 30 minutes total. The second antibody specific to the analyte of interest was labeled with the counter reporter portion, i.e., either SmBiT (FIG. 29B) or LgBiT (FIG. 29A), and added to the assay well in solution. In the presence of analyte, the two antibodies come together and can generate a luminescent signal (FIGS. 29A and B). Antibody-tethered reaction particles, 2^{nd} antibody solution, and IL-2 analyte or negative control were all incubated together for 1.5 hrs, after which Furimazine substrate was added, and luminescent signal was imaged and read on a plate reader. During the protein detection assay process, no washes were required. Both assay systems (FIG. 29A and FIG. 29B) showed signal to background ratios similar or above that of the commercial luminescent Lumit® IL-2 detection system (Promega; FIG. 29C) when comparing RLU on a plate reader. While FIGS. 29A and B were carried out in a Tris/BSA buffer system, FIG. 29D demonstrated (using the FIG. 29B system) use with 10% pooled human serum or plasma.

### Example 12

### Stand Curve and Picogram Levels of Lower Limit of Detection with an Image-Based Homogenous Protein Detection Reaction Particle System

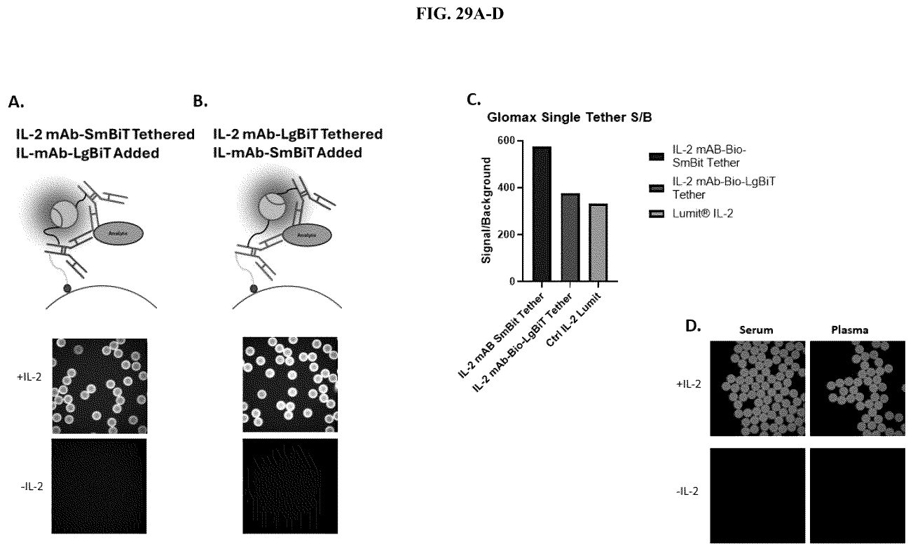

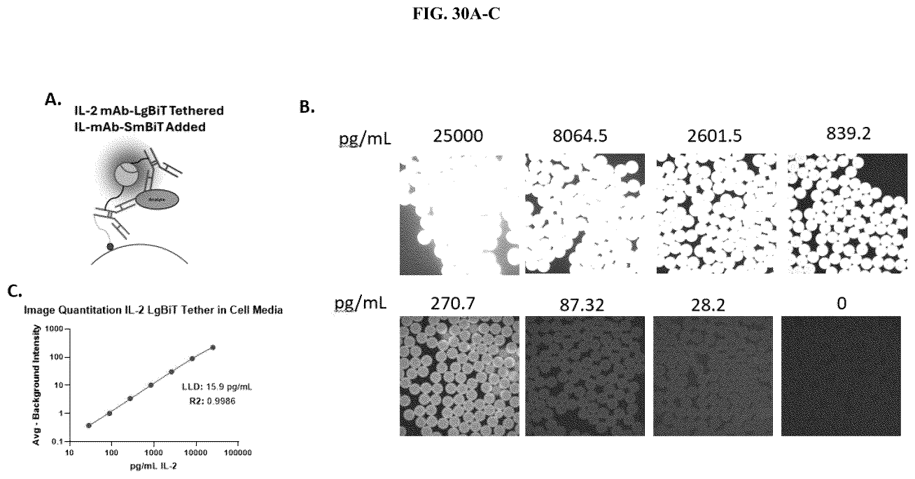

A reaction particle protein detection system was set up as shown FIG. 30A. IL-2 analyte was spiked into cell media to generate a standard curve at the indicated quantities, and the reaction particle protein detection system components were added to the cell media. After 1.5 hrs of incubation, Furimazine substrate was added, reaction particles were imaged (FIG. 30B), and luminescent signal quantified. FIG. 30C provides the IL-2 standard curve using quadratic fit equation, and lower limit of detection calculated using 3 stdev of background showing pg/mL levels of sensitivity as well as R2 value of >0.99.

### Example 13

### Quantitative Multiplexing of Standard Curves on a Homogenous Reaction Particle Protein Detection System

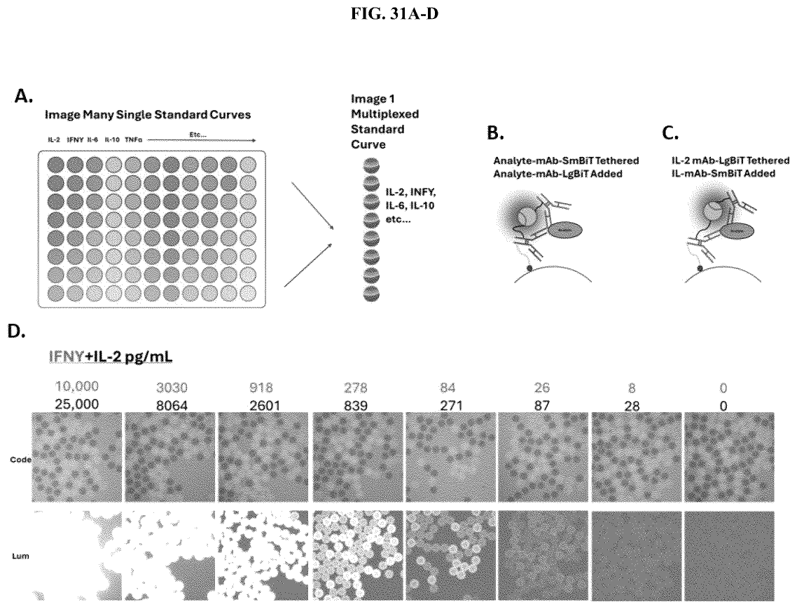

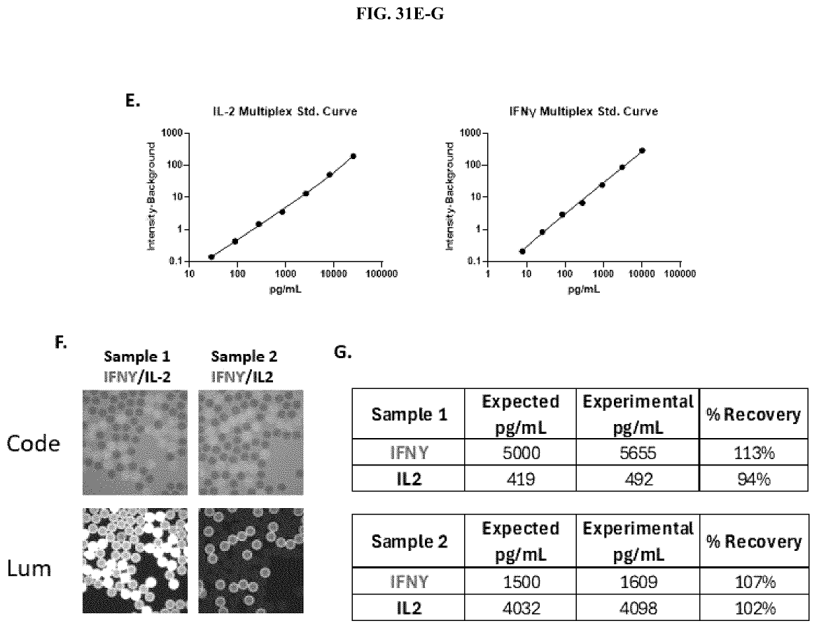

FIG. 31A provides a schematic depiction of the concept of multiplexing a protein standard curve in order to decrease the number of wells needed to be used/imaged. A protein detection reaction particle system for IFNΥ (FIG. 31B) and IL-2 (FIG. 31C) was used. Cell media was spiked with both IFNΥ and IL-2 from increasing to decreasing concentrations to generate a standard curve. The IFNΥ protein detection system used reaction particles that were coded with Cy3, while the IL-2 protein detection system used reaction particles that contained no fluorescent code (FIG. 31D, top panel). Both IFNΥ and IL-2 protein detection systems were added to cell media spiked with both analytes for a multiplex reaction. After a 1.5 hr incubation, Furimazine substrate was added, and brightfield, fluorescent, and luminescent images were taken to obtain reaction particle code and correlating luminescent reporter signal (FIG. 31D). Two different populations of reaction particle codes as well as two different signal intensities of reporter luminescent signal were observed correlating with each analyte IFNΥ or IL-2.

Standard curves for each analyte were extrapolated by quantitating luminescence from each distinct reaction particle code (FIG. 31E). Two samples were made, each spiked with both analytes and IFNΥ and IL-2 at different concentrations. Using the multiplexed standard curves generated, the multiplexed sample concentration was calculated to be within <15% of the expected concentration exemplifying the ability to quantitatively multiplex a homogenous reaction particle protein detection system (FIGS. 31F and G).

### Example 14

### Multiplexing Using a Homogenous Reaction Particle Protein Detection System for Detection of 5 Different Analytes

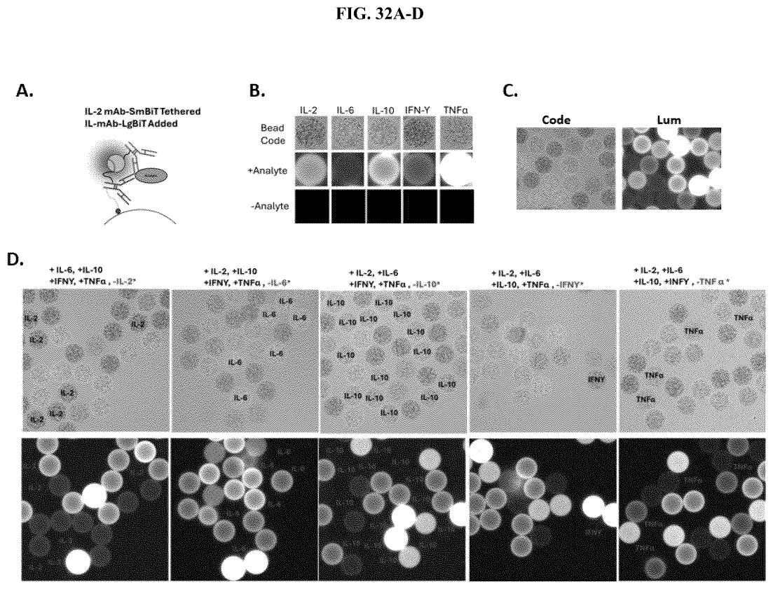

Exemplified in this example is a reaction particle protein detection system for the detection of five different analytes (FIG. 32A). Five reaction particle codes were used, one for each analyte (FIG. 32B top panel): IL-2/Cy3, IL-6/Cy3- and Cy5, IL-10 Cy5 and Cy7, IFNΥ/No label, TNFα/No label, lower optical density (distribution density of magnetic particles within each assay particle). Luminescent signal can be seen for each analyte in the presence (FIG. 32B, middle panel) and absence (FIG. 32B, bottom panel) of the analyte. FIG. 32C shows all five different reaction particles, all five different exogenous antibodies, and all five different analytes added and mixed together, and then incubated and imaged for reaction particle code and corresponding luminescence. In order to show specificity of the multiplex, all five detection reagents were mixed together, and only four of the five analytes were added (FIG. 32D.) The top panel of FIG. 32D shows all five reaction particle codes and labels that contained the missing analyte present in the sample, while the bottom panel of FIG. 32D shows the corresponding reporter luminescent signal with the location of the absent analyte (FIG. 32D) labeled using the above code. For each absent analyte in the sample, a decrease in luminescent signal to near background levels was seen demonstrating the specificity of the signal of each analyte in the five-analyte multiplexed system.

### Example 15

### Raman Detection of Multiplexed Reaction Particles

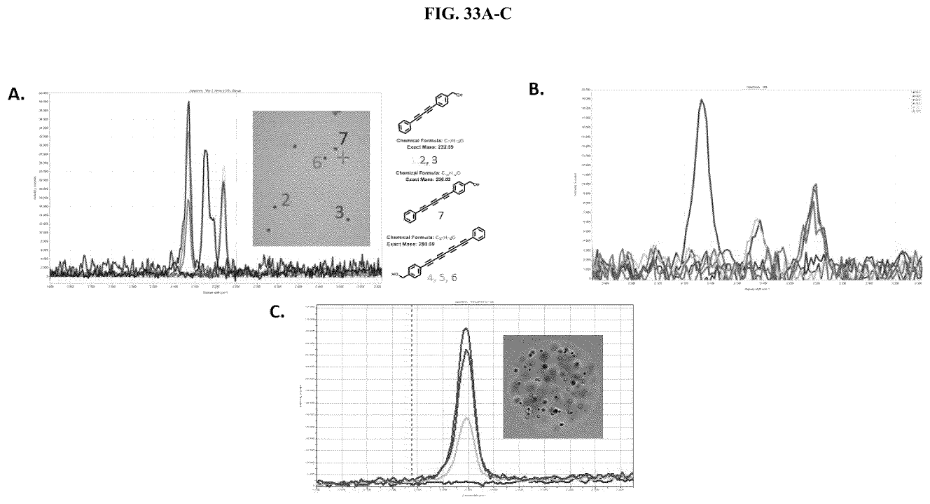

Three Raman reporters were synthesized containing various length polynes that emit at different wavenumbers of the cell silent region of the Raman spectra (FIG. 33A). The three Raman reporters were embedded separately on 10 μm polystyrene beads, and the polystyrene beads were then mixed together and imaged on the Thermo DXR3xi Raman imager 532 nm laser showing the ability to identify the different Raman reporters in the mixture (FIG. 33A).

Three different Raman reporters embedded in 10 μm polystyrene beads and imaged in the presence of NanoBRET® reaction containing 6 nM NanoLuc®-HaloTag® fusion protein, 300 nM JaneliaFluor® 585 HaloTag® ligand, and 20 μM Furimazine showing the ability for the Raman reporters to be imaged in the presence of luminescent, BRET, and fluorescent signals (FIG. 33B).

A Raman reporter was embedded into 1 μm polystyrene beads, and the beads then incorporated into an agarose reaction particle and imaged on a Raman imager. FIG. 33C demonstrates that the signals from three different reaction particles can be detected, including a negative control, which showed no signal at the wavenumber of interest.

### Example 16

### Examination of Different Reaction Bead Material/Composition

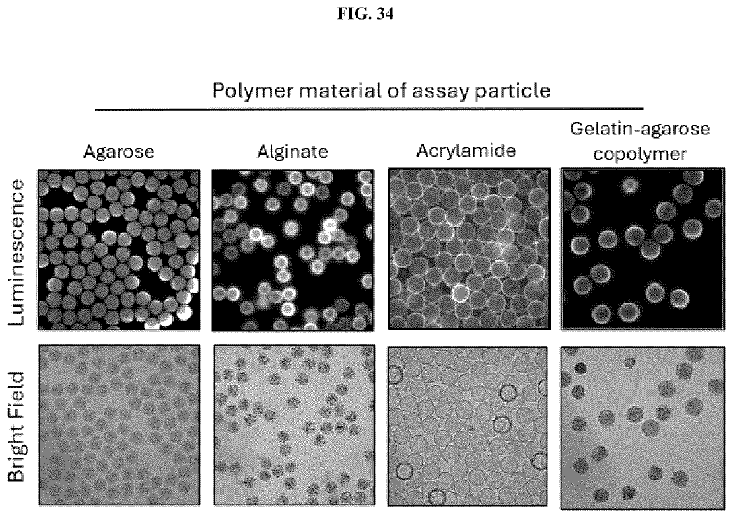

This example demonstrates that bioluminescent reporters are compatible with porous reaction particles made of different polymer matrices. Streptavidin-functionalized reaction particles were loaded with a HaloTag®-NanoLuc® fusion protein covalently labeled with a chloroalkane biotin ligand. The biotin-streptavidin interaction tethers the fusion protein to the beads. Reaction particles were made of natural polymers (agarose and alginate), synthetic polymers (acrylamide; PNIPAAm), and co-polymers (gelatine-agarose) (FIG. 34).

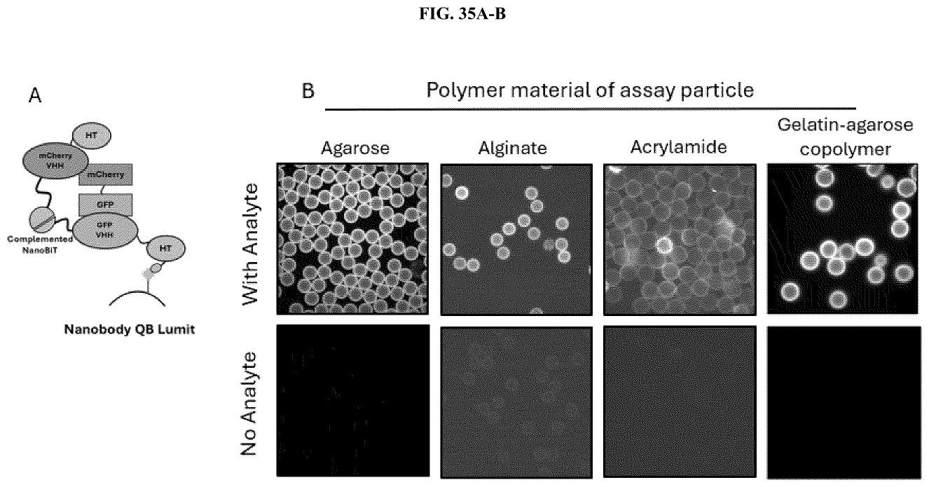

FIG. 35A is a schematic depiction of a NanoBiT® complementation assay using a mCherry-GFP dimer as the analyte, and GFP and mCherry nanobodies (VHH) fused to NanoBiT® components as the detection agents. The GFP-VHH was the first detection agent and was conjugated to the reaction particle using biotin-labelled HaloTag (HT). The mCherry-VHH with unlabelled HT was the second detection agent and was subsequently added homogenously with the analyte. Upon Furimazine substrate addition, the bioluminescent signal was confined to the reaction particle due to analyte dependent complementation of NanoBiT® components, and chemical tethering of the first detection agent leveraging HT.

FIG. 35B provides examples of the bioluminescent complementation assay described above using reaction particles made of natural polymers (agarose and alginate), synthetic polymers (acrylamide; PNIPAAm), and co-polymers (gelatine-agarose).

### Example 17

### Bioluminescent Assay with Cleavage Event

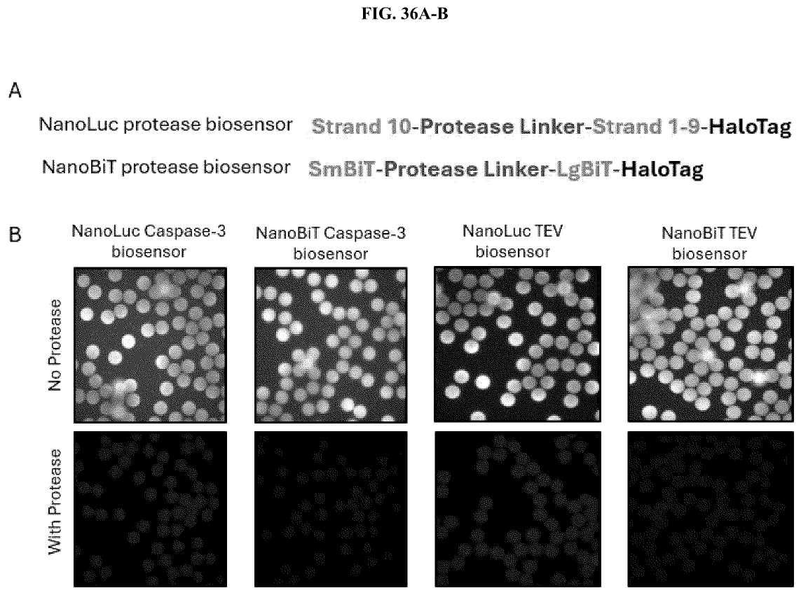

This example demonstrates the compatibility of intramolecular, bioluminescent protease-based cleavage assay with reaction particles of the present invention. FIG. 36A provides a depiction of circularly permuted NanoLuc® and NanoBiT® biosensors in which the binary component bioluminescent reporter is separated by a linker that contains a specific protease recognition site. The protease will recognize the site and cleave the biosensor such that the smaller piece (Strand10 or SmBiT) is cleaved off. In presence of a specific protease, a loss-of-bioluminescent signal is seen. HaloTag is used to chemically conjugate and confine the biosensor to the porous reaction particle.

Separate caspase-3 and TEV proteases biosensors were made as described above. In the absence of a protease, a strong bioluminescent signal was seen. In the presence of the specific protease, the respective biosensor was cleaved, and a loss of bioluminescent signal was seen (FIG. 36B). Due to the inherent low affinity of the binary components, there was a loss of complementation, and a subsequent loss in bioluminescent signal in response to the presence of the respective protease.

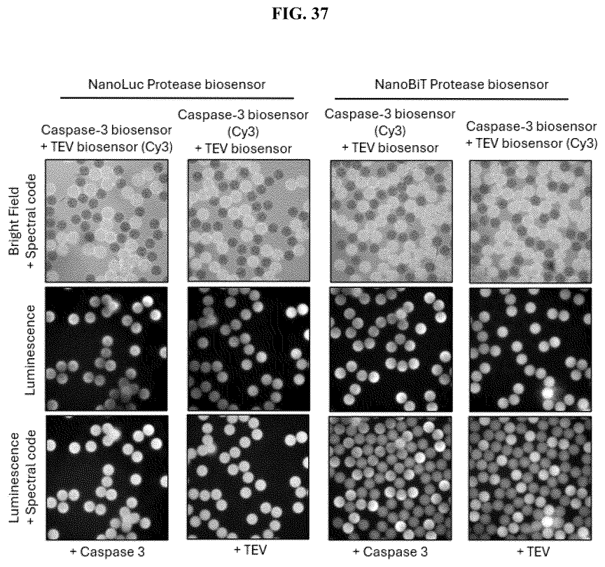

Intramolecular, bioluminescent protease-based cleavage assay can also be multiplexed with reaction particles of the present invention. Protease biosensors were designed as described above. Reaction particles were prepared with either a Caspase-3 protease biosensor or a TEV protease biosensor. The identity of concordant beads onto which each protease biosensor was loaded can be discriminated based on the absence or presence of the spectral fluorescent Cy3 code. Four exemplary assays showed that the biosensors specifically detected the protease's presence as loss of bioluminescent signal observed only in the particles loaded with the relevant, target protease biosensor (FIG. 37). The non-targeted protease biosensor showed no loss-of-signal and retained a bioluminescent signal.

### Example 18

### Compatibility of Different Reporter Modalities with Assay Particles

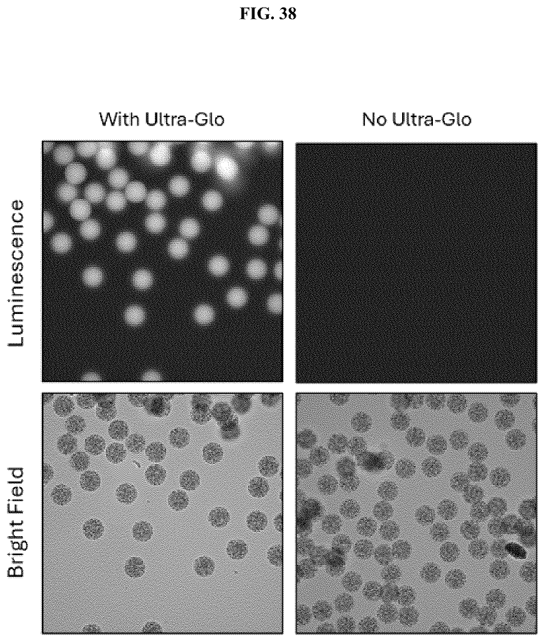

Bioluminescent reporters beyond NanoLuc were demonstrated to be compatible with the reaction particles of the present invention. Streptavidin-functionalized assay particles containing Ultra-Glo® luciferase (Promega) biotinylated via cysteine residues leveraging maleimide were made. Ultra-Glo is an engineered, thermostable form of Photinus pyralis firefly luciferase that uses D-luciferin as a substrate to generate a bioluminescent signal. Luciferin detection reagent was added after addition of Ultra-Glo and showed that specific bioluminescent signal was confined to the porous assay particle (FIG. 38).

### Example 19

### Metabolite Detection Using Reaction Particles

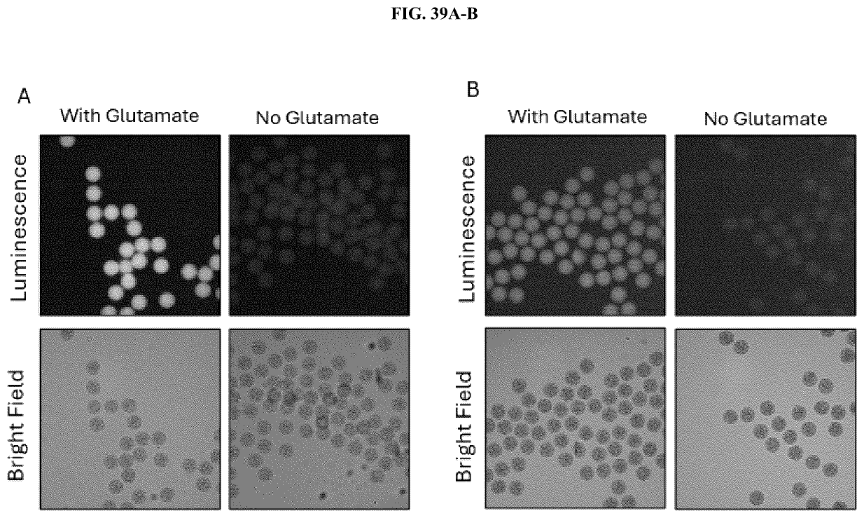

Bioluminescent metabolite assays, such as Glutamate-Glo™ Assay (Promega), are compatible with the reaction particles of the present invention. Streptavidin-functionalized reaction particles were generated with Ultra-Glo® luciferase biotinylated via cysteines (leveraging maleimide chemistry) and glutamate dehydrogenase biotinylated via lysines (leveraging succinimide chemistry). In the presence or absence of glutamate (analyte), luciferin detection agent and reductase enzyme were added concurrently. The three-enzyme chained bioluminescent assay specifically detected the presence of the glutamate metabolite as a bioluminescent signal confined to the porous reaction particles (FIG. 39A).

A modification of the above assay, where all three enzymes were loaded onto the reaction particles, was performed. Streptavidin-functionalized reaction particles were generated with Ultra-Glo® luciferase biotinylated via cysteines (leveraging maleimide chemistry), glutamate dehydrogenase biotinylated via lysines (leveraging succinimide chemistry), and reductase biotinylated via cysteines (leveraging maleimide chemistry. In the presence or absence of glutamate (analyte), luciferin detection agent was added concurrently. The three-enzyme chained bioluminescent assay specifically detected the presence of the glutamate metabolite as a bioluminescent signal confined to the porous reaction particles (FIG. 39B).

### Example 20

### Immunoassay

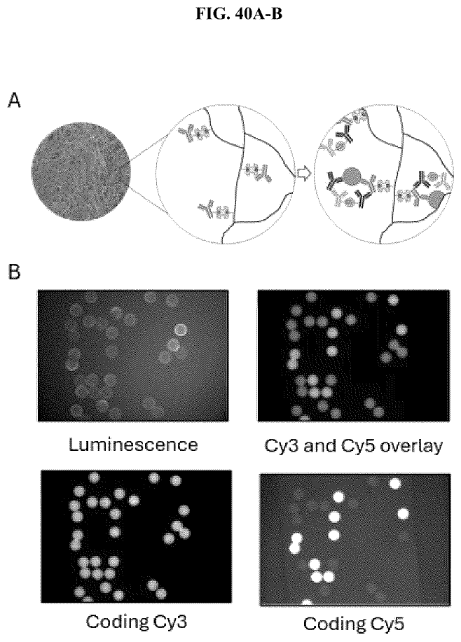

FIG. 40A provides a schematic of an immunoassay utilizing the reaction particles of the present invention. A first primary antibody can be tethered (e.g., by a biotin-streptavidin affinity interaction) to the matrix material of an reaction particle. Then, an analyte can then be added to the reaction particle and bound by the first primary antibody. An untethered primary antibody and secondary antibodies labeled with a luminescent reporter and secondary reporter capable of binding to the first and untethered primary antibodies are added. Upon binding of the primary antibodies to the analyte and binding of the secondary antibodies to the primary antibodies, the luminescent reporter and secondary reporter complement (e.g., via BRET, FRET, structural complementation, etc.). The resultant reaction particles can then be emulsified into a non-aqueous environment using silicone oil with emulsifier (10%). Furimazine substrate can be separately emulsified in an oil-emulsifier mixture, and the emulsified Furimazine delivered to the isolated reaction particles in a non-aqueous environment to produce a detectable, bioluminescent signal in presence of analyte.

Implementation of the immunoassay described above to detect presence of AKT-1 as an analyte in a non-aqueous environment was performed. The immunoassay showed effective complementation of luminescent reporters in the presence of AKT-1. Streptavidin-functionalized porous assay particles with detection reagent were exposed to AKT-1 as target analyte, emulsified, and imaged under steady state conditions in the presence of emulsified Furimazine (FIG. 40B).

### Example 21

### BRET Target Engagement (TE) for Small Molecule/Drug Detection

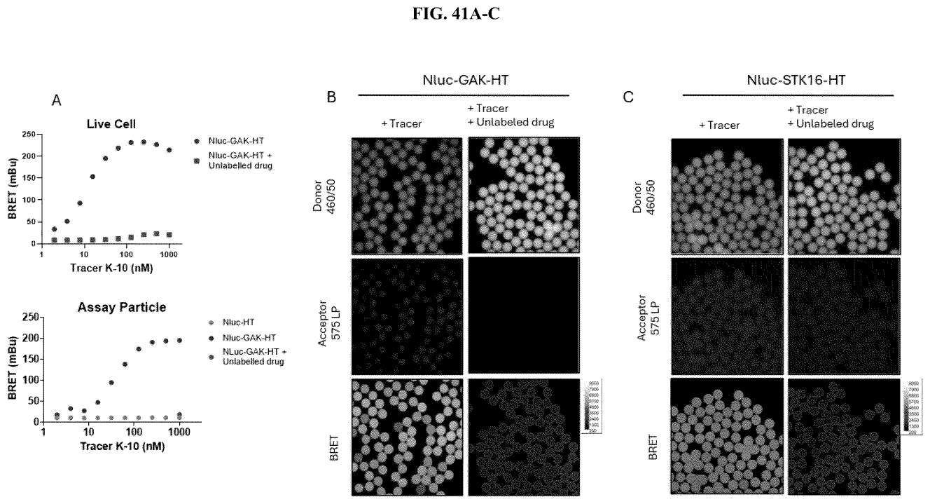

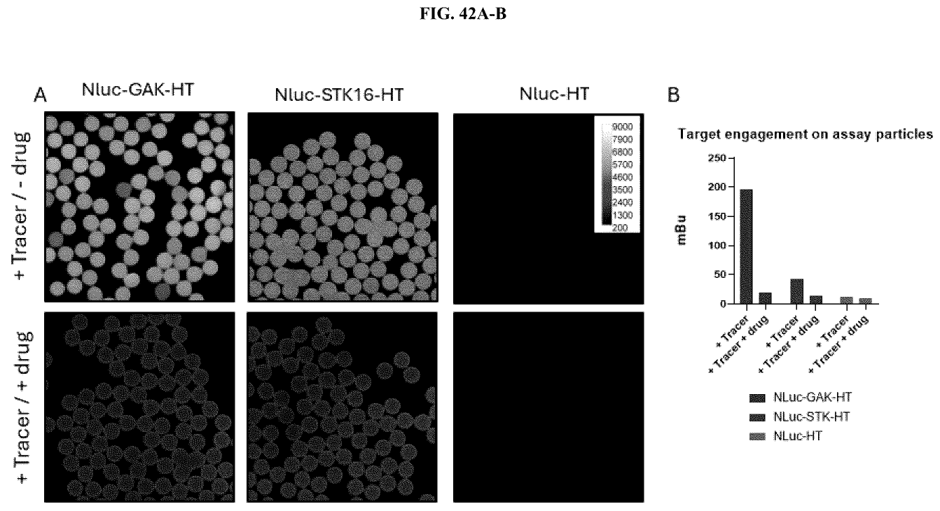

This example demonstrates use of the reaction particles of the present invention for BRET target engagement assays for small molecule/drug detection. NanoBRET® target engagement assay for Cyclin G-Associated Kinase (GAK) was performed in live cells and with reaction particles of the present invention in wells of 96-well plates. In live cells, NanoLuc® (Nluc)-GAK-HaloTag® (HT) fusion protein was overexpressed and labelled with a dose response of GAK-specific K-10 tracer (BRET acceptor). In presence of Furimazine, the Nluc bioluminescent (donor) signal gets transferred to the acceptor and detected as BRET. Upon treatment with unlabeled kinase inhibitor CTx-0294885 (drug), BRET to the K10-tracer was abrogated, as expected. In the porous reaction particles conjugated with chloroalkane, a protein fusion of Nluc-GAK-HT from cell lysates is added onto to the particles via the HT handle. Dose response of K-10 tracer as a BRET acceptor and abrogation of BRET signal in presence of unlabeled drug, as seen in live cells, was recapitulated in the porous reaction particles (FIG. 41A). Excess K-10 tracer was also observed to not lead to an increase in collisional (non-specific) BRET since reaction particles loaded with the fusion construct of Nluc-HT without GAK showed insignificant BRET transfer. This suggests that the BRET signal was specific to the kinase.
Cyclin G-Associated Kinase (GAK) target engagement assay was performed in reaction particles of the invention as described above, and imaged using the GloMax® Galaxy imager. K-10 Tracer acceptor (1 μM) was used to label the Nluc-GAK-HT fusion protein, and effective BRET was seen in presence of Furimazine (FIG. 41B). BRET was abrogated when the assay particles were treated with unlabeled kinase inhibitor (drug: 10 μM of CTx-0294885). The assay was performed in an aqueous format for homogenous delivery of tracer and drug.
Serine/threonine-protein kinase 16 (STK16) target engagement assay was performed in assay particles of the present invention as described above, and imaged using the GloMax® Galaxy imager. K-10 Tracer acceptor (1 μM) was used to label the Nluc-STK16-HT fusion protein, and effective BRET was seen in presence of Furimazine (FIG. 41C). BRET was abrogated when the assay particles were treated with unlabeled kinase inhibitor (drug: 10 μM of CTx-0294885). The assay was performed in an aqueous format for homogenous delivery of tracer and drug.
Cyclin G-Associated Kinase (GAK) and Serine/threonine-protein kinase 16 (STK16) NanoBRET® target engagement assay were performed as described above, and quantified imaging was performed on the GloMax® Galaxy imager. K-10 Tracer acceptor (1 μM) was used to label Nluc-GAK-HT, Nluc-STK16-HT, and Nluc-HT fusion proteins, and effective BRET was seen in presence of Furimazine (FIG. 42A). BRET was abrogated when the assay particles were treated with unlabeled kinase inhibitor (drug: 10 μM of CTx-0294885). The assay was performed in an aqueous format for homogenous delivery of tracer and drug.
Plate-based quantification was performed on the assays described above on a GloMax® Discover plate reader and showed kinase specific inhibition of BRET upon drug treatment (FIG. 42B).

### Example 22

### Different Reaction Particle Size as a System for Coding and Multiplexing

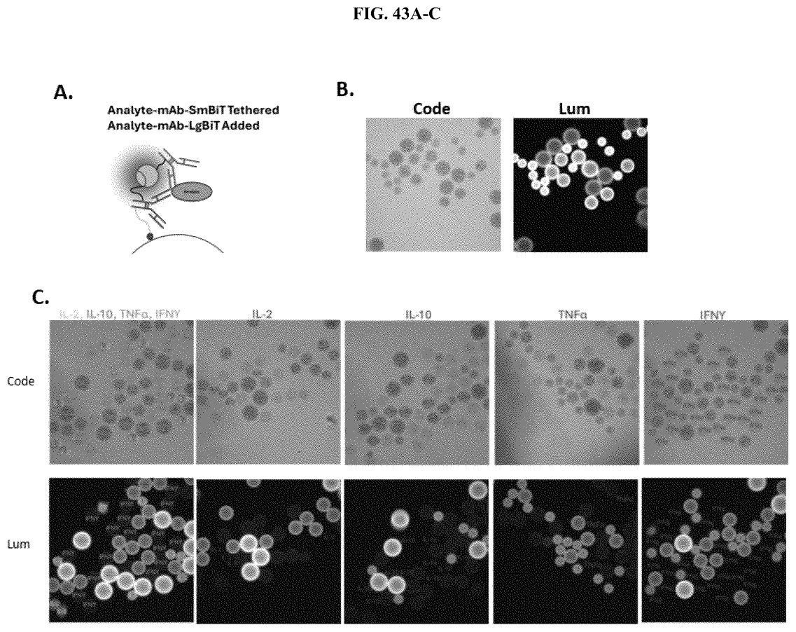

FIG. 43A provides a schematic of the reaction particle protein detection system used in this example. In order to expand on the coding system, reaction particles were made to three different sizes: 70 μm, 90 μm, and 120 μm. The homogenous IL-2 protein reaction particle detection system was employed on particles of all three sizes, and a luminescent signal on each bead size (FIG. 43B). The different particle sizes, as well as a difference of optical density, allowed for a four-analyte multiplex while only image the codes in brightfield (FIG. 43D, top panel). The corresponding codes were as follows: IL-2/70 μm, IL10/90 μm, TNFα/120 μm, IFNΥ/90 μm/low optical density. The first column of FIG. 43D shows all the reaction particles for each analyte, the corresponding exogenous 2^{nd} NanoBiT-labeled antibodies as well as all 4 analytes mixed together. The top panel of FIG. 43D shows the codes while the bottom panel shows the luminescent reporter signal. The subsequent columns of FIG. 43D have all the 4 reaction particle types, all 4 corresponding exogenous NanoBiT-labeled antibodies, but only 3 out of the 4 analytes present to demonstrate specificity. The missing analyte was labeled with its bead code in the top panel of FIG. 43D along with its luminescent signal in the bottom panel of FIG. 43D. A drop to near background levels in luminescent signal from the missing analyte was seen (FIG. 43D, bottom panel) showing the analyte specificity in this multiplex system of 4 analytes.

### Example 23

### Fluorescence and Reaction Particle Size to Generate >20 Codes

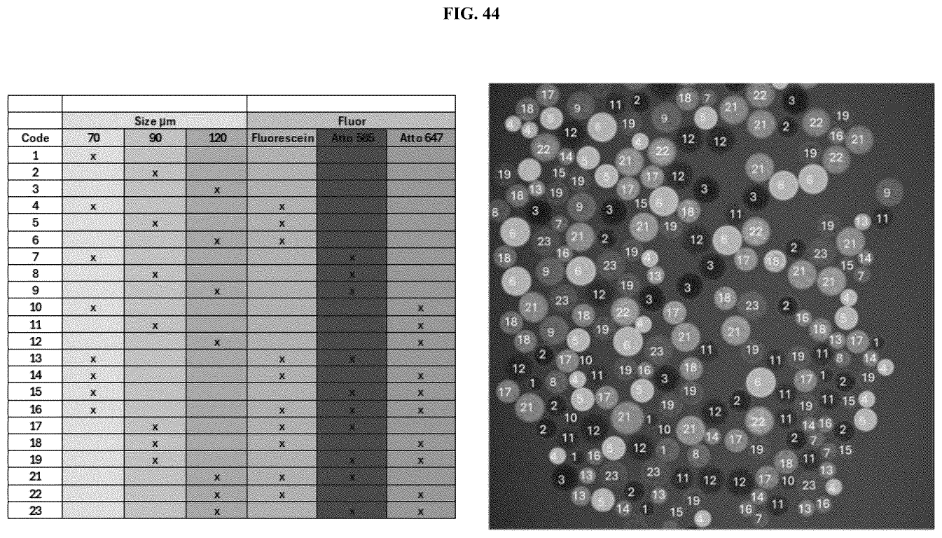

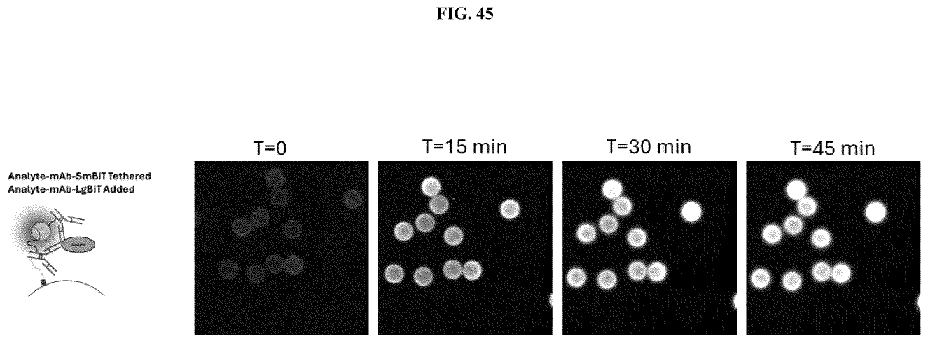

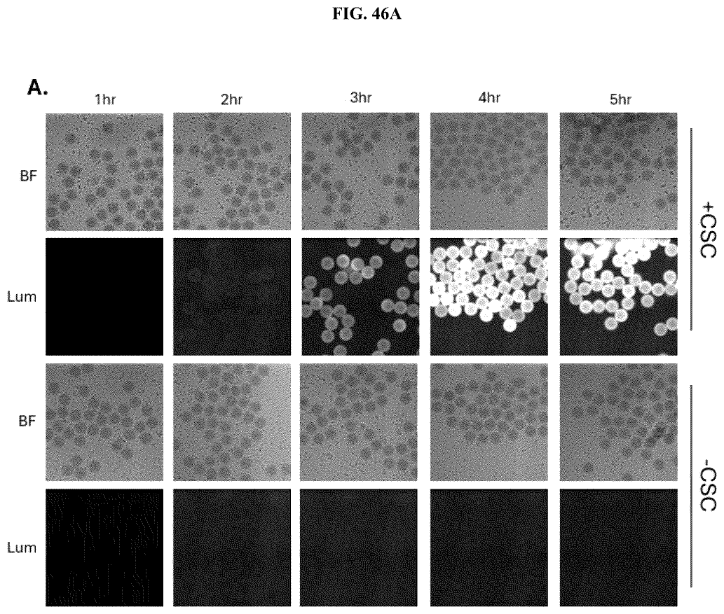

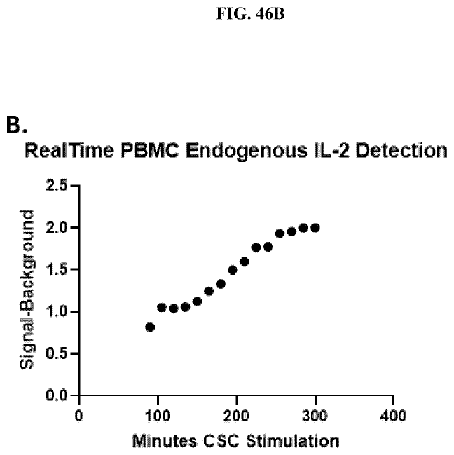

Three particle sizes were labeled with combinations of 3 different fluorescent dyes to generate >20 reaction particle codes (FIG. 44A). All reaction particles were mixed together and imaged in brightfield and fluorescent channels to identify the codes (FIG. 44B).

### Example 24

### Compound Profiling of Kinase Inhibitors Against Cyclin G-Associated Kinase (GAK)

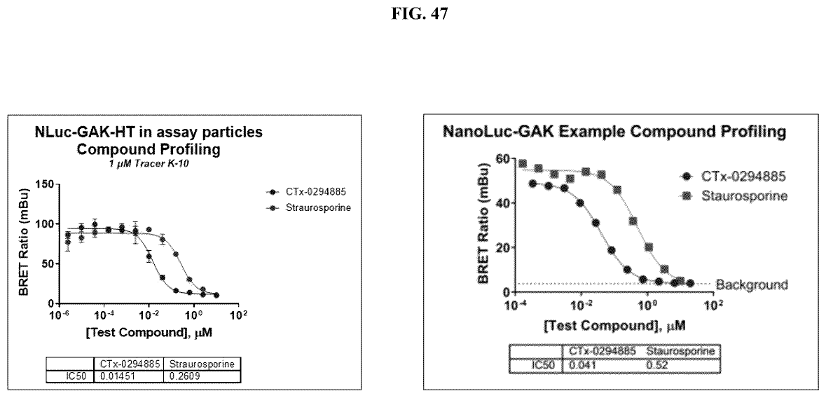

Compound profiling of kinase inhibitors against cyclin G-associated kinase (GAK) in assay particles reproduces both compound ranking and IC₅₀ values observed in cells. NanoLuc (Nluc)-GAK-HaloTag (HT) is overexpressed in HEK293 cells, after which cells are lysed and the Nluc-GAK-HT fusion protein is captured from lysates onto porous assay particles via chloroalkane conjugation to the HaloTag. A K-10 tracer acceptor (1 μM) is used to label Nluc-GAK-HT within the particles. Dose response analyses of unlabeled kinase inhibitors CTx-0294885 and staurosporine show a concentration-dependent loss of BRET signal, consistent with competitive displacement of the K-10 tracer from GAK. The relative IC₅₀ ranking of the two compounds determined in the assay particles mirrors that observed in live-cell measurements, as reported in the commercial NanoBRET® target engagement assay for GAK (FIG. 47). Experiments were performed in 96-well plates using Furimazine as the substrate for the Nluc donor, with luminescence measurements acquired

SEQUENCES

SEQ ID NO: 1-NANOLUC-
MKHHHHHHAIAMVFTLEDFVGDWRQTAGYNLDQVLEQGGVSSLFQNLGV
SVTPIQRIVLSGENGLKIDIHVIIPYEGLSGDQMGQIEKIFKVVYPVDD
HHFKVILHYGTLVIDGVTPNMIDYFGRPYEGIAVFDGKKITVTGTLWNG
NKIIDERLINPDGSLLFRVTINGVTGWRLCERILAV
SEQ ID NO: 2-full-length NANOBIT-
MVFTLEDFVGDWEQTAAYNLDQVLEQGGVSSLLQNLAVSVTPIQRIVRS
GENALKIDIHVIIPYEGLSADQMAQIEEVFKVVYPVDDHHFKVILPYGT
LVIDGVTPNMLNYFGRPYEGIAVFDGKKITVTGTLWNGNKIIDERLITP
DGSMLFRVTINSVTGYRLFEEIL
SEQ ID NO: 3-HiBIT-VSGWRLFKKIS
SEQ ID NO: 4-SmBIT-VTGYRLFEEIL
SEQ ID NO: 5-LgBiT-
MVFTLEDFVGDWEQTAAYNLDQVLEQGGVSSLLQNLAVSVTPIQRIVRS
GENALKIDIHVIIPYEGLSADQMAQIEEVFKVVYPVDDHHFKVILPYGT
LVIDGVTPNMLNYFGRPYEGIAVFDGKKITVTGTLWNGNKIIDERLITP
DGSMLFRVTINSHHHHHH
SEQ ID NO: 6-LgTrip-
MVFTLEDFVGDWEQTAAYNLDQVLEQGGVSSLLQNLAVSVTPIQRIVRS
GENALKIDIHVIIPYEGLSADQMAQIEEVFKVVYPVDDHHFKVILPYGT
LVIDGVTPNMLNYFGRPYEGIAVFDGKKITVTGTLWNGNKIIDERLITP
D
SEQ ID NO: 7-SmTrip9-GSMLFRVTINS
SEQ ID NO: 8-HALOTAG-
MAEIGTGFPFDPHYVEVLGERMHYVDVGPRDGTPVLFLHGNPTSSYVWR
NIIPHVAPTHRCIAPDLIGMGKSDKPDLGYFFDDHVRFMDAFIEALGLE
EVVLVIHDWGSALGFHWAKRNPERVKGIAFMEFIRPIPTWDEWPEFARE
TFQAFRTTDVGRKLIIDQNVFIEGTLPMGVVRPLTEVEMDHYREPFLNP
VDREPLWRFPNELPIAGEPANIVALVEEYMDWLHQSPVPKLLFWGTPGV
LIPPAEAARLAKSLPNCKAVDIGPGLNLLQEDNPDLIGSEIARWLSTLE
ISG

1-290. (canceled)

291. A composition comprising a population of assay particles;
wherein the assay particles are porous, comprise a matrix material, and comprise an interstitial void volume suitable to receive a sample;
wherein each assay particle comprises a target-specific luminescent detection agent tethered to the matrix material; wherein the target-specific luminescent detection agent of each assay particle comprises: (i) a target-specific interaction moiety, and (ii) a luminescent reporter capable of producing a luminescent signal individually or in combination with other reagents; wherein interaction of the target-specific interaction moiety with the target analyte results in a change in the luminescent signal;
wherein the population of assay particles comprises distinct subsets of assay particles; wherein the assay particles of each subset comprise a target-specific interaction moiety that is subset specific; wherein each subset comprises a different target-specific interaction moiety; and wherein all of the assay particles of the population comprise the same luminescent reporter capable of producing the same luminescent signal; and
wherein the assay particles of each subset comprise a subset-specific physical characteristic that is the same for the assay particles within each subset and distinct from the assay particles of other subsets; wherein the subset-specific physical characteristic is independent of the luminescent signal of the luminescent reporter.

292. The composition of claim 291, wherein the target-specific interaction moiety of each subset is specific for a different target analyte.

293. The composition of claim 291, further comprising a secondary detection agent, wherein the secondary detection agent comprises (i) a secondary reporter, and (ii) (A) a secondary target-specific interaction moiety, or (B) a target mimetic.

294. The composition of claim 293, wherein the luminescent reporter is a bioluminescent protein, the secondary reporter is a fluorophore, and the luminescent reporter and the secondary reporter are a bioluminescence resonance energy transfer (BRET) pair.

295. The composition of claim 293, wherein the luminescent reporter is a first fluorophore protein, the secondary reporter is a second fluorophore, and the luminescent reporter and the secondary reporter are a Förster resonance energy transfer (FRET) pair.

296. The composition of claim 293, wherein the luminescent reporter and secondary reporter are first and second components of a complementation-dependent bioluminescent complex

297. The composition of claim 293, wherein the secondary detection agent comprises a secondary target-specific interaction moiety, and wherein the target-specific interaction moiety and the secondary target-specific interaction moiety bind to separate locations on the target.

298. The composition of claim 293, wherein the secondary detection agent comprises a target mimetic, and wherein the target mimetic is suitable for binding to the target-specific interaction moiety and competing with the target analyte for binding to the target-specific interaction moiety.

299. The composition of claim 291, wherein the assay-particle-identifying physical characteristic comprises one or more of particle dimensions, particle shape, particle morphology, emitted wavelength, color, signal strength, signal intensity, wavenumber, distribution or composition of magnetic particles within the assay particle, optical density, distribution of optical density, distribution of optical features, and a spectral signature.

300. The composition of claim 299, wherein spectral signature is selected from a fluorescent signal, a colorimetric signal, a Raman signal, an infrared signal, a chemiluminescent signal, and a bioluminescent signal.

301. A method of conducting a multiplex assay, the method comprising:
(a) contacting a sample with the composition of claim 1, and allowing the interstitial void volumes of the assay particles to receive the sample;
(b) allowing the target-specific interaction moiety each of the assay particles to interact with a target analyte, if present, in the sample;
(c) detecting the luminescent signal from the luminescent reporter in each of the assay particles; and
(d) detecting the subset-specific physical characteristic of each of the assay particles.

302. The method of claim 301, further comprising maintaining fluid communication between the interstitial void volume and the exterior of the assay particles during steps (b) through (d).

303. The method of claim 301, further comprising identifying the assay-particle-identifying physical characteristic from each of the distinct assay particles, thereby identifying each of the distinct assay particles.

304. The method of claim 303, further comprising correlating the luminescent signal from the luminescent reporter to interaction of the target analyte and target-specific interaction moiety based on the identification of the assay particles.

305. The method of any one of claim 301, further comprising a step, prior to step (c), of contacting the distinct assay particles with a substrate for the luminescent reporter of the distinct assay particles.

306. The method of claim 301, wherein the detecting the luminescent signal from the luminescent reporter in each of the assay particles is real-time detection.

307. The method of claim 301, wherein the detecting the luminescent signal from the luminescent reporter in each of the assay particles is end-point detection.


SEQUENCE LISTING (8 sequences)

SEQ ID NO: 1 - protein, 183 aa, Synthetic construct
MKHHHHHHAIAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAV

SEQ ID NO: 2 - protein, 170 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSVTGYRLFEEIL

SEQ ID NO: 3 - protein, 11 aa, Synthetic construct
VSGWRLFKKIS

SEQ ID NO: 4 - protein, 11 aa, Synthetic construct
VTGYRLFEEIL

SEQ ID NO: 5 - protein, 165 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 6 - protein, 148 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPD

SEQ ID NO: 7 - protein, 11 aa, Synthetic construct
GSMLFRVTINS

SEQ ID NO: 8 - protein, 297 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG